SEQUENCE LISTING (167 sequences)

SEQ ID NO: 1 - DNA, 16 bp, artificial
  nucleobase sequence / LNA oligonucleotide sequence
gctgagtgattactta

SEQ ID NO: 2 - DNA, 16 bp, artificial
  nucleobase sequence / LNA oligonucleotide sequence
atgctgagtgattact

SEQ ID NO: 3 - DNA, 16 bp, artificial
  nucleobase sequence / LNA oligonucleotide sequence
agatgctgagtgatta

SEQ ID NO: 4 - DNA, 16 bp, artificial
  nucleobase sequence / LNA oligonucleotide sequence
aaagatgctgagtgat

SEQ ID NO: 5 - DNA, 16 bp, artificial
  nucleobase sequence / LNA oligonucleotide sequence
gaaaagatgctgagtg

SEQ ID NO: 6 - DNA, 16 bp, artificial
  nucleobase sequence / LNA oligonucleotide sequence
aggaaaagatgctgag

SEQ ID NO: 7 - DNA, 16 bp, artificial
  nucleobase sequence / LNA oligonucleotide sequence
tcaggaaaagatgctg

SEQ ID NO: 8 - DNA, 16 bp, artificial
  nucleobase sequence / LNA oligonucleotide sequence
tgtcaggaaaagatgc

SEQ ID NO: 9 - DNA, 16 bp, artificial
  nucleobase sequence / LNA oligonucleotide sequence
attgtcaggaaaagat

SEQ ID NO: 10 - DNA, 16 bp, artificial
  nucleobase sequence / LNA oligonucleotide sequence
aaattgtcaggaaaag

SEQ ID NO: 11 - DNA, 16 bp, artificial
  nucleobase sequence / LNA oligonucleotide sequence
aaaaattgtcaggaaa

SEQ ID NO: 12 - DNA, 16 bp, artificial
  nucleobase sequence / LNA oligonucleotide sequence
aaaaaaattgtcagga

SEQ ID NO: 13 - DNA, 16 bp, artificial
  nucleobase sequence / LNA oligonucleotide sequence
acaaaaaaattgtcag

SEQ ID NO: 14 - DNA, 16 bp, artificial
  nucleobase sequence / LNA oligonucleotide sequence
ctacaaaaaaattgtc

SEQ ID NO: 15 - DNA, 16 bp, artificial
  nucleobase sequence / LNA oligonucleotide sequence
aactacaaaaaaattg

SEQ ID NO: 16 - DNA, 16 bp, artificial
  nucleobase sequence / LNA oligonucleotide sequence
ataactacaaaaaaat

SEQ ID NO: 17 - DNA, 16 bp, artificial
  nucleobase sequence / LNA oligonucleotide sequence
cataactacaaaaaaa

SEQ ID NO: 18 - DNA, 16 bp, artificial
  nucleobase sequence / LNA oligonucleotide sequence
cacataactacaaaaa

SEQ ID NO: 19 - DNA, 16 bp, artificial
  nucleobase sequence / LNA oligonucleotide sequence
gtcacataactacaaa

SEQ ID NO: 20 - DNA, 16 bp, artificial
  nucleobase sequence / LNA oligonucleotide sequence
aagtcacataactaca

SEQ ID NO: 21 - DNA, 12 bp, artificial
  nucleobase sequence / LNA oligonucleotide sequence
cacataactaca

SEQ ID NO: 22 - DNA, 16 bp, artificial
  nucleobase sequence / LNA oligonucleotide sequence
caaagtcacataacta

SEQ ID NO: 23 - DNA, 12 bp, artificial
  nucleobase sequence / LNA oligonucleotide sequence
gtcacataacta

SEQ ID NO: 24 - DNA, 16 bp, artificial
  nucleobase sequence / LNA oligonucleotide sequence
aacaaagtcacataac

SEQ ID NO: 25 - DNA, 12 bp, artificial
  nucleobase sequence / LNA oligonucleotide sequence
aagtcacataac

SEQ ID NO: 26 - DNA, 16 bp, artificial
  nucleobase sequence / LNA oligonucleotide sequence
aaaacaaagtcacata

SEQ ID NO: 27 - DNA, 12 bp, artificial
  nucleobase sequence / LNA oligonucleotide sequence
caaagtcacata

SEQ ID NO: 28 - DNA, 16 bp, artificial
  nucleobase sequence / LNA oligonucleotide sequence
acaaaacaaagtcaca

SEQ ID NO: 29 - DNA, 12 bp, artificial
  nucleobase sequence / LNA oligonucleotide sequence
aacaaagtcaca

SEQ ID NO: 30 - DNA, 16 bp, artificial
  nucleobase sequence / LNA oligonucleotide sequence
ttacaaaacaaagtca

SEQ ID NO: 31 - DNA, 16 bp, artificial
  nucleobase sequence / LNA oligonucleotide sequence
atttacaaaacaaagt

SEQ ID NO: 32 - DNA, 16 bp, artificial
  nucleobase sequence / LNA oligonucleotide sequence
aaatttacaaaacaaa

SEQ ID NO: 33 - DNA, 16 bp, artificial
  nucleobase sequence / LNA oligonucleotide sequence
ataaatttacaaaaca

SEQ ID NO: 34 - DNA, 16 bp, artificial
  nucleobase sequence / LNA oligonucleotide sequence
tataaatttacaaaac

SEQ ID NO: 35 - DNA, 16 bp, artificial
  nucleobase sequence / LNA oligonucleotide sequence
gacattttacttattt

SEQ ID NO: 36 - DNA, 16 bp, artificial
  nucleobase sequence / LNA oligonucleotide sequence
aagacattttacttat

SEQ ID NO: 37 - DNA, 16 bp, artificial
  nucleobase sequence / LNA oligonucleotide sequence
acaagacattttactt

SEQ ID NO: 38 - DNA, 16 bp, artificial
  nucleobase sequence / LNA oligonucleotide sequence
tcacaagacattttac

SEQ ID NO: 39 - DNA, 16 bp, artificial
  nucleobase sequence / LNA oligonucleotide sequence
tttcacaagacatttt

SEQ ID NO: 40 - DNA, 16 bp, artificial
  nucleobase sequence / LNA oligonucleotide sequence
gtttcacaagacattt

SEQ ID NO: 41 - DNA, 16 bp, artificial
  nucleobase sequence / LNA oligonucleotide sequence
tgtttcacaagacatt

SEQ ID NO: 42 - DNA, 16 bp, artificial
  nucleobase sequence / LNA oligonucleotide sequence
ttgtttcacaagacat

SEQ ID NO: 43 - DNA, 16 bp, artificial
  nucleobase sequence / LNA oligonucleotide sequence
ttttgtttcacaagac

SEQ ID NO: 44 - DNA, 16 bp, artificial
  nucleobase sequence / LNA oligonucleotide sequence
cattttgtttcacaag

SEQ ID NO: 45 - DNA, 16 bp, artificial
  nucleobase sequence / LNA oligonucleotide sequence
agcattttgtttcaca

SEQ ID NO: 46 - DNA, 16 bp, artificial
  nucleobase sequence / LNA oligonucleotide sequence
aaagcattttgtttca

SEQ ID NO: 47 - DNA, 16 bp, artificial
  nucleobase sequence / LNA oligonucleotide sequence
taaaaagcattttgtt

SEQ ID NO: 48 - DNA, 16 bp, artificial
  nucleobase sequence / LNA oligonucleotide sequence
gttaaaaagcattttg

SEQ ID NO: 49 - DNA, 16 bp, artificial
  nucleobase sequence / LNA oligonucleotide sequence
atgttaaaaagcattt

SEQ ID NO: 50 - DNA, 16 bp, artificial
  nucleobase sequence / LNA oligonucleotide sequence
tggatgttaaaaagca

SEQ ID NO: 51 - DNA, 16 bp, artificial
  nucleobase sequence / LNA oligonucleotide sequence
tatggatgttaaaaag

SEQ ID NO: 52 - DNA, 16 bp, artificial
  nucleobase sequence / LNA oligonucleotide sequence
ttatatggatgttaaa

SEQ ID NO: 53 - DNA, 16 bp, artificial
  nucleobase sequence / LNA oligonucleotide sequence
gctttatatggatgtt

SEQ ID NO: 54 - DNA, 16 bp, artificial
  nucleobase sequence / LNA oligonucleotide sequence
tagctttatatggatg

SEQ ID NO: 55 - DNA, 16 bp, artificial
  nucleobase sequence / LNA oligonucleotide sequence
gatagctttatatgga

SEQ ID NO: 56 - DNA, 16 bp, artificial
  nucleobase sequence / LNA oligonucleotide sequence
tagatagctttatatg

SEQ ID NO: 57 - DNA, 16 bp, artificial
  nucleobase sequence / LNA oligonucleotide sequence
tatagatagctttata

SEQ ID NO: 58 - DNA, 16 bp, artificial
  nucleobase sequence / LNA oligonucleotide sequence
tatatagatagcttta

SEQ ID NO: 59 - DNA, 16 bp, artificial
  nucleobase sequence / LNA oligonucleotide sequence
tatatatagatagctt

SEQ ID NO: 60 - DNA, 16 bp, artificial
  nucleobase sequence / LNA oligonucleotide sequence
aaaaacatttgttttc

SEQ ID NO: 61 - DNA, 16 bp, artificial
  nucleobase sequence / LNA oligonucleotide sequence
tcaaaaacatttgttt

SEQ ID NO: 62 - DNA, 16 bp, artificial
  nucleobase sequence / LNA oligonucleotide sequence
gttcaaaaacatttgt

SEQ ID NO: 63 - DNA, 16 bp, artificial
  nucleobase sequence / LNA oligonucleotide sequence
atgttcaaaaacattt

SEQ ID NO: 64 - DNA, 16 bp, artificial
  nucleobase sequence / LNA oligonucleotide sequence
aaatgttcaaaaacat

SEQ ID NO: 65 - DNA, 16 bp, artificial
  nucleobase sequence / LNA oligonucleotide sequence
ttaaatgttcaaaaac

SEQ ID NO: 66 - DNA, 16 bp, artificial
  nucleobase sequence / LNA oligonucleotide sequence
ttttaaatgttcaaaa

SEQ ID NO: 67 - DNA, 16 bp, artificial
  nucleobase sequence / LNA oligonucleotide sequence
gtttttaaatgttcaa

SEQ ID NO: 68 - DNA, 16 bp, artificial
  nucleobase sequence / LNA oligonucleotide sequence
aagtttttaaatgttc

SEQ ID NO: 69 - DNA, 16 bp, artificial
  nucleobase sequence / LNA oligonucleotide sequence
tgaagtttttaaatgt

SEQ ID NO: 70 - DNA, 16 bp, artificial
  nucleobase sequence / LNA oligonucleotide sequence
ctgaagtttttaaatg

SEQ ID NO: 71 - DNA, 16 bp, artificial
  nucleobase sequence / LNA oligonucleotide sequence
tctgaagtttttaaat

SEQ ID NO: 72 - DNA, 16 bp, artificial
  nucleobase sequence / LNA oligonucleotide sequence
catctgaagtttttaa

SEQ ID NO: 73 - DNA, 16 bp, artificial
  nucleobase sequence / LNA oligonucleotide sequence
aacatctgaagttttt

SEQ ID NO: 74 - DNA, 16 bp, artificial
  nucleobase sequence / LNA oligonucleotide sequence
ctaacatctgaagttt

SEQ ID NO: 75 - DNA, 16 bp, artificial
  nucleobase sequence / LNA oligonucleotide sequence
ttctaacatctgaagt

SEQ ID NO: 76 - DNA, 16 bp, artificial
  nucleobase sequence / LNA oligonucleotide sequence
ctttctaacatctgaa

SEQ ID NO: 77 - DNA, 16 bp, artificial
  nucleobase sequence / LNA oligonucleotide sequence
aactttctaacatctg

SEQ ID NO: 78 - DNA, 16 bp, artificial
  nucleobase sequence / LNA oligonucleotide sequence
tcaactttctaacatc

SEQ ID NO: 79 - DNA, 16 bp, artificial
  nucleobase sequence / LNA oligonucleotide sequence
tttcaactttctaaca

SEQ ID NO: 80 - DNA, 16 bp, artificial
  nucleobase sequence / LNA oligonucleotide sequence
ctttcaactttctaac

SEQ ID NO: 81 - DNA, 16 bp, artificial
  nucleobase sequence / LNA oligonucleotide sequence
cctttcaactttctaa

SEQ ID NO: 82 - DNA, 16 bp, artificial
  nucleobase sequence / LNA oligonucleotide sequence
ttaacctttcaacttt

SEQ ID NO: 83 - DNA, 16 bp, artificial
  nucleobase sequence / LNA oligonucleotide sequence
cattaacctttcaact

SEQ ID NO: 84 - DNA, 16 bp, artificial
  nucleobase sequence / LNA oligonucleotide sequence
gctgagtgattactta

SEQ ID NO: 85 - DNA, 16 bp, artificial
  nucleobase sequence / LNA oligonucleotide sequence
atgctgagtgattact

SEQ ID NO: 86 - DNA, 16 bp, artificial
  nucleobase sequence / LNA oligonucleotide sequence
agatgctgagtgatta

SEQ ID NO: 87 - DNA, 16 bp, artificial
  nucleobase sequence / LNA oligonucleotide sequence
aaagatgctgagtgat

SEQ ID NO: 88 - DNA, 16 bp, artificial
  nucleobase sequence / LNA oligonucleotide sequence
gaaaagatgctgagtg

SEQ ID NO: 89 - DNA, 16 bp, artificial
  nucleobase sequence / LNA oligonucleotide sequence
aggaaaagatgctgag

SEQ ID NO: 90 - DNA, 16 bp, artificial
  nucleobase sequence / LNA oligonucleotide sequence
tcaggaaaagatgctg

SEQ ID NO: 91 - DNA, 16 bp, artificial
  nucleobase sequence / LNA oligonucleotide sequence
tgtcaggaaaagatgc

SEQ ID NO: 92 - DNA, 16 bp, artificial
  nucleobase sequence / LNA oligonucleotide sequence
attgtcaggaaaaaat

SEQ ID NO: 93 - DNA, 16 bp, artificial
  nucleobase sequence / LNA oligonucleotide sequence
aaattgtcaggaaaag

SEQ ID NO: 94 - DNA, 16 bp, artificial
  nucleobase sequence / LNA oligonucleotide sequence
aaaaattgtcaggaaa

SEQ ID NO: 95 - DNA, 16 bp, artificial
  nucleobase sequence / LNA oligonucleotide sequence
aaaaaaattgtcagga

SEQ ID NO: 96 - DNA, 16 bp, artificial
  nucleobase sequence / LNA oligonucleotide sequence
acaaaaaaaatgtcag

SEQ ID NO: 97 - DNA, 16 bp, artificial
  nucleobase sequence / LNA oligonucleotide sequence
ctacaaaaaaattgtc

SEQ ID NO: 98 - DNA, 16 bp, artificial
  nucleobase sequence / LNA oligonucleotide sequence
aactacaaaaaaattg

SEQ ID NO: 99 - DNA, 16 bp, artificial
  nucleobase sequence / LNA oligonucleotide sequence
ataactacaaaaaaat

SEQ ID NO: 100 - DNA, 16 bp, artificial
  nucleobase sequence / LNA oligonucleotide sequence
cataactacaaaaaaa

SEQ ID NO: 101 - DNA, 16 bp, artificial
  nucleobase sequence / LNA oligonucleotide sequence
cacataactacaaaaa

SEQ ID NO: 102 - DNA, 16 bp, artificial
  nucleobase sequence / LNA oligonucleotide sequence
gtcacataactacaaa

SEQ ID NO: 103 - DNA, 16 bp, artificial
  nucleobase sequence / LNA oligonucleotide sequence
aagtcacataactaca

SEQ ID NO: 104 - DNA, 12 bp, artificial
  nucleobase sequence / LNA oligonucleotide sequence
cacataactaca

SEQ ID NO: 105 - DNA, 16 bp, artificial
  nucleobase sequence / LNA oligonucleotide sequence
caaagtcacataacta

SEQ ID NO: 106 - DNA, 12 bp, artificial
  nucleobase sequence / LNA oligonucleotide sequence
gtcacataacta

SEQ ID NO: 107 - DNA, 16 bp, artificial
  nucleobase sequence / LNA oligonucleotide sequence
aacaaagtcacataac

SEQ ID NO: 108 - DNA, 12 bp, artificial
  nucleobase sequence / LNA oligonucleotide sequence
aagtcacataac

SEQ ID NO: 109 - DNA, 16 bp, artificial
  nucleobase sequence / LNA oligonucleotide sequence
aaaacaaagtcacata

SEQ ID NO: 110 - DNA, 12 bp, artificial
  nucleobase sequence / LNA oligonucleotide sequence
caaagtcacata

SEQ ID NO: 111 - DNA, 16 bp, artificial
  nucleobase sequence / LNA oligonucleotide sequence
acaaaacaaagtcaca

SEQ ID NO: 112 - DNA, 12 bp, artificial
  nucleobase sequence / LNA oligonucleotide sequence
aacaaagtcaca

SEQ ID NO: 113 - DNA, 16 bp, artificial
  nucleobase sequence / LNA oligonucleotide sequence
ttacaaaacaaagtca

SEQ ID NO: 114 - DNA, 16 bp, artificial
  nucleobase sequence / LNA oligonucleotide sequence
atttacaaaacaaagt

SEQ ID NO: 115 - DNA, 16 bp, artificial
  nucleobase sequence / LNA oligonucleotide sequence
aaatttacaaaacaaa

SEQ ID NO: 116 - DNA, 16 bp, artificial
  nucleobase sequence / LNA oligonucleotide sequence
ataaatttacaaaaca

SEQ ID NO: 117 - DNA, 16 bp, artificial
  nucleobase sequence / LNA oligonucleotide sequence
tataaatttacaaaac

SEQ ID NO: 118 - DNA, 16 bp, artificial
  nucleobase sequence / LNA oligonucleotide sequence
gacattttacttattt

SEQ ID NO: 119 - DNA, 16 bp, artificial
  nucleobase sequence / LNA oligonucleotide sequence
aagacattttacttat

SEQ ID NO: 120 - DNA, 16 bp, artificial
  nucleobase sequence / LNA oligonucleotide sequence
acaagacattttactt

SEQ ID NO: 121 - DNA, 16 bp, artificial
  nucleobase sequence / LNA oligonucleotide sequence
tcacaagacattttac

SEQ ID NO: 122 - DNA, 16 bp, artificial
  nucleobase sequence / LNA oligonucleotide sequence
tttcacaagacatttt

SEQ ID NO: 123 - DNA, 16 bp, artificial
  nucleobase sequence / LNA oligonucleotide sequence
gtttcacaagacattt

SEQ ID NO: 124 - DNA, 16 bp, artificial
  nucleobase sequence / LNA oligonucleotide sequence
tgtttcacaagacatt

SEQ ID NO: 125 - DNA, 16 bp, artificial
  nucleobase sequence / LNA oligonucleotide sequence
ttgtttcacaagacat

SEQ ID NO: 126 - DNA, 16 bp, artificial
  nucleobase sequence / LNA oligonucleotide sequence
ttttgtttcacaagac

SEQ ID NO: 127 - DNA, 16 bp, artificial
  nucleobase sequence / LNA oligonucleotide sequence
cattttgtttcacaag

SEQ ID NO: 128 - DNA, 16 bp, artificial
  nucleobase sequence / LNA oligonucleotide sequence
agcattttgtttcaca

SEQ ID NO: 129 - DNA, 16 bp, artificial
  nucleobase sequence / LNA oligonucleotide sequence
aaagcattttgtttca

SEQ ID NO: 130 - DNA, 16 bp, artificial
  nucleobase sequence / LNA oligonucleotide sequence
taaaaagcattttgtt

SEQ ID NO: 131 - DNA, 16 bp, artificial
  nucleobase sequence / LNA oligonucleotide sequence
gttaaaaagcattttg

SEQ ID NO: 132 - DNA, 16 bp, artificial
  nucleobase sequence / LNA oligonucleotide sequence
atgttaaaaagcattt

SEQ ID NO: 133 - DNA, 16 bp, artificial
  nucleobase sequence / LNA oligonucleotide sequence
tggatgttaaaaagca

SEQ ID NO: 134 - DNA, 16 bp, artificial
  nucleobase sequence / LNA oligonucleotide sequence
tatggatgttaaaaag

SEQ ID NO: 135 - DNA, 16 bp, artificial
  nucleobase sequence / LNA oligonucleotide sequence
ttatatggatgttaaa

SEQ ID NO: 136 - DNA, 16 bp, artificial
  nucleobase sequence / LNA oligonucleotide sequence
gctttatatggatgtt

SEQ ID NO: 137 - DNA, 16 bp, artificial
  nucleobase sequence / LNA oligonucleotide sequence
tagctttatatggatg

SEQ ID NO: 138 - DNA, 16 bp, artificial
  nucleobase sequence / LNA oligonucleotide sequence
gatagctttatatgga

SEQ ID NO: 139 - DNA, 16 bp, artificial
  nucleobase sequence / LNA oligonucleotide sequence
tagatagctttatatg

SEQ ID NO: 140 - DNA, 16 bp, artificial
  nucleobase sequence / LNA oligonucleotide sequence
tatagatagctttata

SEQ ID NO: 141 - DNA, 16 bp, artificial
  nucleobase sequence / LNA oligonucleotide sequence
tatatagatagcttta

SEQ ID NO: 142 - DNA, 16 bp, artificial
  nucleobase sequence / LNA oligonucleotide sequence
tatatatagatagctt

SEQ ID NO: 143 - DNA, 16 bp, artificial
  nucleobase sequence / LNA oligonucleotide sequence
aaaaacatttgttttc

SEQ ID NO: 144 - DNA, 16 bp, artificial
  nucleobase sequence / LNA oligonucleotide sequence
tcaaaaacatttgttt

SEQ ID NO: 145 - DNA, 16 bp, artificial
  nucleobase sequence / LNA oligonucleotide sequence
gttcaaaaacatttgt

SEQ ID NO: 146 - DNA, 16 bp, artificial
  nucleobase sequence / LNA oligonucleotide sequence
atgttcaaaaacattt

SEQ ID NO: 147 - DNA, 16 bp, artificial
  nucleobase sequence / LNA oligonucleotide sequence
aaatgttcaaaaacat

SEQ ID NO: 148 - DNA, 16 bp, artificial
  nucleobase sequence / LNA oligonucleotide sequence
ttaaatgttcaaaaac

SEQ ID NO: 149 - DNA, 16 bp, artificial
  nucleobase sequence / LNA oligonucleotide sequence
ttttaaatgttcaaaa

SEQ ID NO: 150 - DNA, 16 bp, artificial
  nucleobase sequence / LNA oligonucleotide sequence
gtttttaaatgttcaa

SEQ ID NO: 151 - DNA, 16 bp, artificial
  nucleobase sequence / LNA oligonucleotide sequence
aagtttttaaatgttc

SEQ ID NO: 152 - DNA, 16 bp, artificial
  nucleobase sequence / LNA oligonucleotide sequence
tgaagtttttaaatgt

SEQ ID NO: 153 - DNA, 16 bp, artificial
  nucleobase sequence / LNA oligonucleotide sequence
ctgaagtttttaaatg

SEQ ID NO: 154 - DNA, 16 bp, artificial
  nucleobase sequence / LNA oligonucleotide sequence
tctgaagtttttaaat

SEQ ID NO: 155 - DNA, 16 bp, artificial
  nucleobase sequence / LNA oligonucleotide sequence
catctgaagtttttaa

SEQ ID NO: 156 - DNA, 16 bp, artificial
  nucleobase sequence / LNA oligonucleotide sequence
aacatctgaagttttt

SEQ ID NO: 157 - DNA, 16 bp, artificial
  nucleobase sequence / LNA oligonucleotide sequence
ctaacatctgaagttt

SEQ ID NO: 158 - DNA, 16 bp, artificial
  nucleobase sequence / LNA oligonucleotide sequence
ttctaacatctgaagt

SEQ ID NO: 159 - DNA, 16 bp, artificial
  nucleobase sequence / LNA oligonucleotide sequence
ctttctaacatctgaa

SEQ ID NO: 160 - DNA, 16 bp, artificial
  nucleobase sequence / LNA oligonucleotide sequence
aactttctaacatctg

SEQ ID NO: 161 - DNA, 16 bp, artificial
  nucleobase sequence / LNA oligonucleotide sequence
tcaactttctaacatc

SEQ ID NO: 162 - DNA, 16 bp, artificial
  nucleobase sequence / LNA oligonucleotide sequence
tttcaactttctaaca

SEQ ID NO: 163 - DNA, 16 bp, artificial
  nucleobase sequence / LNA oligonucleotide sequence
ctttcaactttctaac

SEQ ID NO: 164 - DNA, 16 bp, artificial
  nucleobase sequence / LNA oligonucleotide sequence
cctttcaactttctaa

SEQ ID NO: 165 - DNA, 16 bp, artificial
  nucleobase sequence / LNA oligonucleotide sequence
ttaacctttcaacttt

SEQ ID NO: 166 - DNA, 16 bp, artificial
  nucleobase sequence / LNA oligonucleotide sequence
cattaacctttcaact

SEQ ID NO: 167 - DNA, 35073 bp, homo sapiens
tctgattggtgagcgatggtggtaggtaaaaccagtcttagagtcatggcagttcattttagcagctattaggtaaaactggtcttagggatacagaaggctggttcagcagttggacttgtggaaaatttaattcttgaagcagatgctgtgtgccccgaatgcttcttccccctggcccttcaactctgatttagttgagtatttcaagaatgacccaatttatgtaatcaactttcacaggtatacatgtcttaaactttaaacagatgttttgggttttgttgttgttgtttttgagacggagtctcactctgttgtccaagctggagtgtagtggtgtgatctcggctcactgcaacctccgcctccagggtcaagtgattctccagcctcagcctcctgaatagctgggattacaggcgcccgccaccacgcccagctaatttttgtatttttagtagagatggggtggggtttcaccatgttggccaggctggtcttgaactcctgacatcaagtgttctgctcacctcagcctctgaaagtgctgggattacaggcgtgagccactgcgcccggcagtctttccttcttttttttttttttttttttttttttaatgacatggggtcttactttattactcaggctggtctcaaacttctggcctcaaggaatcttcccaccttggcctcccaaattgctgggattacaggcataagtcatcatgcctggctacaaacagatattttcaataagaggataaaagttcatttccccatactttgctaacatcaaatgttattaattcctaatagttttgccaaactgagaggaaaatggtatgttagtttttctgggttttctttctttttaattttttttcttttttattcatcgcaacactattcacgatttttttattttttattttatttatttatttatttttttttgagacaaggtctccctatgttgcccaggctggtcttgtacccctgggctcaaaggatcctcctgcctcagcctcccaaagtgctaggattacaggcatgagtcaccacgcctggttcacaatttctttttgtttttaccaaaggcaggtatattcctgaaattttttgtttttttgttttttttttgagatgaagtctcaccctgtcactcagactggagtgcactggcacgatctcagctcactgcaacctccgcttcctaggttcaagcgattctcctgcctcagtcttcaaagtagctaggattataggcgccgcaaccatgctcagctaatttttgtatttttagtagagacaggatttcaccatgttggccagggtgatctcaaatcctgacctcaagtgatccgcctgcctcagcctcccaaagtgctgggattactggcatgagccaccgtgccaggccctgaaatgttatcttagttattaatttgcaattccttggctctagaggttgggcatcttctcagatctctagtggacatttggattttctttttggtgaactgtccagtttttctctctgctttacaatctttattatatgcaatcttcacatgtaggtactaccatttttttagtttgtttttgaaacagcatattgctctgttgcccaggctggagcacggtggcaaaaacatggcttactgcagcctttgacctccttggctcaagtactcctcctgtctcagcctcctgagtagctggtaccacaagcccataccaccatgcccagctaatttatttttgtagagatggggcctgaccatgttacttgggctcaaatgatcctctcccactcagcctcccaaagtgctaggattacaggcatgagccaccatacttggccccttttttttttttttttttttttttttttttttttttttgagacacagtcttgctctgtctcacaggctggtgtgcagtggcacgatctcagctcattgcaacctccacctcccagtttcaagtgatttttgtgcctcagcctccctagtagctgagattacaggcatgcaccaccatgcctggctgactttcatatctttagtgttgccatgttggctaggctggtctcaaactcctgacctcatgtgatccacctgcctcggactcccacagtgctgggattacaggtgttagccaccaccccgacattatttgaaacttttattttatcatgagagagttccaggagtcaactgaagagagatttttggtatgaaaattacatatgcaaaaagactgattccagtacatgaaattaaattcaacatttacattaaatgccttcaaatatggtaaaatggtttcttttggcagtttacctcattatgttttgaatgatttgtctatcatatgaaataacttttataaatatagtaactcaggcctgggcacagcggctcaagtgggaggactgcttaagcaaccgagtttgagaccagcgtggacaacatggggagaccccttctctcccaaaaaatagctgagcatggcagcgcactgttaaaggaaacagagtttctttggtgggtgattaaaatgttctggagttagatagtagtgatggttgcacaaccttgtgaatatattaaggtttccgctctatctaccattcaattgtactctctaaaacgattaattctatagtatatcaattatatctctaaataataaaaacaaaaagaaatggctgggtgcggtggctcatgcctgtaatcgcagcactttgggaggctgaggcgggcggatcacaaggtcaggagtttgagaccaacctgaccaacatggcaaaacccagtctctactaaaaatacaaaaattagctgggtgtggtggcacacgcctgtaatcccaggtactcgggaagctgagacaggagaatcacttgaacccgggaggcagaggttgcagtgagccactgcactccagtctgggtggcagagcgagactccgtctcaaaaagaaaaaaattaaaaaacaaaaagaaacctggttctatattttgtttaaatttatttttttaaccatcatgtaatatgtccaggtaatttgtttaaattttgacatcaaatgcaattgtgagaatttttatgattcagaaaaatctaagcaagctttataaaaacatacttttttttttacttttttttttttttctgagacacagcctcactctgtcgcccaggctggagggcaggttttcatgtttatctgtgagatgtacctttggcacattactttcctgacatgagatttaaatttttttttttatcttgtgacaatttaacttttttgacacataaaaattgtacatatttatttgtttgagatggagtcgcactctgtcactcaggctggagtgcagtggcgtgatcttggctcactgcaacctccgcctcccgagttcaagtgattctcctggctcagcctcccaagcagctgtcattacaggcctgcaccaccacacccggctgatttttgtatttttaggagaaacagggtttcaccatgttggccaggctggtcttgaagtcctgacctcaagtgatccacccaccttggcctcccaaagtgctgggattataggcatgagccaccgtaccagacccctaaaaattgtatatatttaaggtgtaccatttgatgtttagatatacattgtgaaatgattacattccacatattacctctacagagttaccatttttgtacacttggtcaacatcatcccattctccccttcctccacagatatttcttgtatactatatagaagccaagggtattttgggggaagagctcaaagttcctttcgtggagttaaaaatatatatatactatgtacatataagccatttagcaaccctagatgcttaataaagaatactggaggcccggtgtggtggctcacacctgtaatcccagcactttgggaggccgaggcggtcggattacgaggtcaggagttcaagaccagcctggccaacatggtgaaaccccatctttactaaaaatacaaaaattagccgggtgtggtggtgggcgcctgtaatcccagctactcggggggctgaggcagaattgcttgaacctgggaggcagaggttgcagtgagctgagatcacgccactgcattccagcctgggtgacagagcaatactctgtcgcaaaaaaaaaaaagaatactggaggctgggcgaggtggctcacacctgtaatcccagcattttgggatgccagaggcgggcggaatatcttgagctcaggagttcgagaccagcctacacaatatgctccaaacgccgcctctacaaaacatacagaaactagccgggtgtggtggcgtgcccctgtggtcctagctacttgggaggttgaggcgggaggatcgcttgagctcgggaggtcgaggctgcaatgagccgagatggtgccactgcactctgacgacagagcgagactccgtctcaaaacaaacaacaaataaggttgggggatcaaatatcttctagtgtttaaggatctgccttccttcctgcccccatgtttgtctttccttgtttgtctttatatagatcaagcaggttttaaattcctagtaggagcttacatttacttttccaagggggagggggaataaatatctacacacacacacacacacacacacacacacacacactggagttcgagacgaggcctaagcaacatgccgaaaccccgtctctactaaatacaaaaaatagctgagcttggtggcgcacgcctatagtcctagctactggggaggctgaggtgggaggatcgcttgagcccaagaagtcgaggctgcagtgagccgagatcgcgccgctgcactccagcctgagcgacagggcgaggctctgtctcaaaacaaacaaacaaaaaaaaaaggaaaggaaatataacacagtgaaatgaaaggattgagagaaatgaaaaatatacacgccacaaatgtgggagggcgataaccactcgtagaaagcgtgagaagttactacaagcggtcctcccggccaccgtactgttccgctcccagaagccccgggcggcggaagtcgtcactcttaagaagggacggggccccacgctgcgcacccgcgggtttgctatggcgatgagcagcggcggcagtggtggcggcgtcccggagcaggaggattccgtgctgttccggcgcggcacaggccaggtgaggtcgcagccagtgcagtctccctattagcgctctcagcacccttcttccggcccaactctccttccgcagcctcgggacagcatcaagtcgatccgctcactggagttgtggtccgcgtttttctacgtcttttcccactccgttccctgcgaaccacatccgcaagctccttcctcgagcagtttgggctccttgatagcgttgagtggaggccctgccgcgacttggcagtagcttattttgttcactcctctctggctggtgtgggggaggtgggggcattaggccagggtgaagcaggggaaccacttaggagtctgttaagatgatctgaacttcagaacaagatgttattaacagagtgaaagtatttggattctgggtatattttgaaatcggaggcaacaggtttttcagatagattcgataacggaggttatcctgaatagttgaaaagataaagttgccttttgctgaggtgggaaagagaagattgccagtagagcaggtttctcaggagttcagtcttgggcatagcatggtaggggtgaatttggctggagtgagttggagagtaggagaagagaaatccaaggcaacatttgaccagcctgggcaacatagtgtgactccgagtctgcaaaaattagacgggtgttgtggtgcgcgtctgtggtctcagctacctggaaggttcaggccttggaaggctcagggaggtggaggctgcagtgatctgtgattgcgcctctgcactccagcctgggcgacagagccagaccctgtcttaaaacaaaataaacggccgggcgcggtggctcaagcctgtaatcccagcactttgggaggccgaggcggccggatcacaaggtcaggagatcgagaccatcctggctaacacggtgaaaccccgtctctactacaaatacaaaaaattagccgggcgtggtgacgggcgcctgtagtcccagctactcgggaggctgaggcaggagaatgtcatgaagccgggaggcggagcttgcagtgagccgagatcgcgccactgcactccagcctgggcgatagagcaagactccgtctcaaataaataaataaataaataaataaataataaaaacatcggtaggcatatttcaaggaattctatttaaaaaaaatttttttagagacaagttcgctctctgtggcccaggctggagtacagtggcatgatcctagcccatggcagcgttgatctcttggcctcaagcgaccctcctttggagtcgctgggcctaaaggagtgagccaccacgaaattttattataaatggagggtagagaaattgggcaataaatggagggggaagtgagttaagaggaattttaattatgtgtgtgtggttttaaaagaggggggtcttgctctgttgcccaggctgctggggtgccagtggcgcaatcatgaatcactacagccttggactcctggcctcaagctatcctcccacctctgcctcccaaagtactgggattactagtgtgagccactgcactaagataggagcaacatgtttcagcatgtttgtgggttgataggaaagatgagaatgggaaagttgatgtcggaaagaagacaatggctagagcaatgtcctagagtaggtaagaagggatggatttggcctttgttggaaacattagcggttcttttggtgacagctatatagttaacacatctatgatacgtgaatgggcagataggatggcaggagattttgaaagttctcttgattcttactgttctcttagtgaaagaagcaaggttatcagctagaagctgggatgggagaggaaagagaagatgggaagtagatagttctttagaagagtgggcaagggttggactagggaagtttagtggaaatattgctaggcaacataaagagcctacttgagattcgtggtcatgagttgaaggagaccagacagcaagattgtgtatgagggcacccacagagtaaatggagagttgaaattaatgcagttgtgattttaccacgtggatatgaagaagtgagggggagaagtacaaaggagttctcttaatgattgaccatggaatttaagctggctaagaaaggaagtgagaggccgggcgcggtggctcacgcctgtaatcccagcactttgggagactgaggtgggtggattacctgaggtcaggagtttgagaccaacctggccgatatggcgaaaccccatctctaataaaaatacagaaaaattagccgggaatggtggcaggtgcctgtaatcccagctactcaagaggctgtggcaggagtatcccttggacccaggaggtggaggttgcagtgagccgagatcacgccactgtactccagcctggacgatatagtgagacttcacctcaaaaaaaaaaaaaaagaaaggaagtgaggattttaagaccctgagagacagtttaaaaagtgggaggatcggccgggcgctgtggctgacacctgtaatcccagcactttgggaggccgagttgggcagatcacaaggtcaggagttcgagaccagcctggccaatatggtgaaaccttgtctctactaaaaatacaaaaattagccgggcatggtgtcacgtgtctataatcccagctactcgggaggctgaggcagaaaaattgcttgaacctgggaggcagaggttgcagacagctgagatcactccattgcactccagcctgggcaacaagagcaaaactttgtctttaaaaaaaaaaaaaaaaaaagaatacaaaaattagccgggcgtggtggcgcgtgcctataatcccagctacttgggaggctgaggcaggagaatcagttgaacacgggaggcgaggtttgcagtgagccgagattgcgccactgcactccagcctgggcgacagagcaggactcctcttggaaaaaaaaaattagctgggcatggtggcaggtgcctgtagtctcagctactagggaggctgaggcaggaaaatcacttgaacccgggatgtggagtttgcagtgacccgagatcgtgccactgtactccatcctgggcgacaaaatgagactctgcctcaaaaaaaaaaaaaaaaaaaagtgggaggatcaatgtactgccagtcctaatgaagtggaatgattgtccccatcaaatcactagtaggagtaagttgcagagcctagaaggtgatggttaagagagtgggattcttgaaactgcatttatggagaggttgtggttattggttataataaataaatacagttgaagtgagtgagtagctgagatttggggatgtatcagttcattcttacactgctacaaagacatacctgagaccaggtatttataaagataagaggtttaatcagctcacagttctgctgcctgtacaggcttctcttgtggaggcctaaggaaacttacagtcatggtggaaggtgaaggggaaacaagcacagtcttcacatggccagcaggagagagagagaagggggaagtgctacatactttaaaacaaccagatcttgtgagaacgcttatcaggaaacagcacttggggatggtgctaaatcattagaaatcacccccatgatccagtcgcctcctaccatgcccacctccaacactggggatcacaattcagcatgagatttgggtaggaacacagagctgcaccacatcagaggatgtacaagattgtggtggagaggagtttagagacctgcaaatatagggtaattgaagggatcatctacatggatatttaaatcaccaaaaattatgacaggagtagtgttggagagagaactgcgatgtaaacattaaggaatgaggaagagtgactcggtaggctgtaggtgactgcaataggaaacgataatagactgtgagtctggtgacaagattttccttctttctttttttccccccccccgagacagggcctctttttgttgcccaggtgggagtgcagtggcgcgatcacggctcactacaacctcctcccaagctcaagggattctcccacttcagcctctcaagtagctggaactacaggtgctgaccaccatgcctggctactttttgtcaggattttcaaggctgggaattttgagaggggaatggaggagaataatctgaaagtgcaagtaaggagcagggaagatttcttttttcttttttttttttttttttgagtcggagtctggctcagtcgcccaggctggagtgcagtggcgagatctccgctcactgcaagctccgcctcccgtgttcacgccattctcctccttcagcctcccgagtagctgggactacaggcgcccgccaccacgcccagctaattgtttttttgtatttttagtagagacggggtttcaccgtgttagccaggatggtctcaatctcctgactttgtgatccgcccaccccggcctcccaaagcgcttgggattacaggcgtgagccaccgcgccagccagagcagggaagatttcttccccacatctccagtaggtacagtgatatgaagtgtgtggaggagaaaagaggaaacatctatcatttgagatggctgcgaaaggaaaaggcatcctcagggagctagattttacttagagcaagaaatgaagggatgattcagaggttaaaagagtggattttatgaattactcaagggagcacagtggaagtttcaggaagtggtaggagaaggtagaagatggcagggtgttgggaataatttgagaaatctgagctactggaaatgactgagaatcagatataaaggcagtcctggtggtccgttctggctgccgttgctgtgtaacgaatctgccaaaacttagtggcttgaaacaacaaagaacattttattatctctcattgtttctgtgggttaggaatttgtgagagccgtgctgggcagttttcgtgcggctgtctcgtggttgcacctacatagttgctagagctacagtagctggggactgagcagctagggattggcaggctatctctttttttcatgtagtctcatgaagatttctttatgtggtttcaatgtgtgggctggtttggatttccttatagcatggtggcctcagttggattgctgttttgtgatccttttcatccctccttgtcctgtccccagacaaccactgatctactttctgtcaccatagattagcctgcatttttaagaatttttataaacgtggaatgatagagtaccttttttgtcacgtttcttttatttatcatagctattttgattttcatccattttattgctgagtagtatcccattgcatgtatatactatactgtattcattcgcttgcttgtgaacatttgggctttttccagtttgggactgttaacaagtagagccactatgaatattagtgtataagacttcatatagccaaggctggcagatcgcttgagcccaggagtttgagaccagcctgggaaacatggtgaaacctctatttttattttaaaatcaaaaattaaaaattttctataaaaaattttaaagaagactttgtatagacatacgctttcatttttcttgagtgaatacttaggtctcagggtagatgtattttaagtctttaaggagctgtcaaactcttcctcaaagtggtggttgtaccatgttactttttaatataacagagattaattgagcaaagaaaaattcaaaagttggacagcccccacaactaaataggttcagaacagctcccccattttgcattttgaccagcaatgtatgaaagttccatttgctcagtgtccctgcaaacacctggtatggtcagtctttttaattttaggcattataatagatatagtggcttcttgtgattttaattagcatttcctaatgaccagtgctgctgttgatcatttcatgagtgtatttgccatccgtatatcttttttggtgaagtgtctattcaaatcatttgggttttttttttttttgttttttttttttggagacagtgtctcactctgtcacccaggctgttgtgcagtggtgcaatcacacagcctactgcagcctccacctcctgcgctcagtcttcttgtctcagccttctgagtagctgaaattacgagcacacgccacaatgcctggctaattttttaaaattttgtagaaacaaggtctcattatgttgcctgggcttgtcgtgaactcctgggctcaagcaatcttcctgcctcagcctcccaaagattgggattgcaagtatgagccactgcacccggccaacttacccatcttttaattgaatttttttgttgttgaggtttgagagttcttcatgtttgctgggtacaatatctttatcagataggtaacttgcatgtattttctcccggtttacactttggtttttcattttgttaacaacgtctttttaagaacagaaaatcttaattttgctgaaatctaatttttcagttttttctttgatggttttgagagaggaggtaaaaaaagactaggtaagccgatagttagacagagtcctcggtagaacttcccttctaacaaaaagcagcccaagaaatcacttctcttctaacaaggagcagcctggaagatcgggctgtaaacatgtataaggaagcagctctggcacagagggggagcttcctgggtaatcagcaagcttcacatacgtaaggtgggtatgtgaagtaaacacagtatgtgaagtaaacacagtggaccttagtacatactcagataaggaagctggaagcttgcatgttgtgagttgttggggttgcctgcagctgcacggagagaaaggggtacctggggccaggcatgtccaccatggtggctccacctccccttatttagcacatgcacaataggaaagagataagcaatgtggagtagctcaggccaaggacctgcctgcataataaaaggttggggtgggggatgccagagattcacgctctgtgcagatggcaacacctggtcctaactggttttttgctccctatgtgtagataagctacccccttcccattagctcatttataaaaatgcttgcatttcactgtggaatgggaactcttttcaggacctctctctgcaggagagagctagtctctttcttttgcctattaaacttctgctctagcctcacacccttggtgtgtcagcgtccttgatttcctcagcgtgagaccaagaacctcgggtgccaccccaggcaacaaggccatttcagtttgttcttttgttataggcaatccatgatcacagatttttctctcttttttttttttacacagtttagagttttagttttacacttaggtctgtaatccattttgtattaattcttatatgtggctcagtgtaggtggaaatttggtttgtttttgcataaggatttccaatagttttaccaccatttcttgaaactactatgctttctctattaaaccacatttgtaactttagttaaaatcagtcacatatatcacagggctatttctgactctcaattctgttacattgtctattagtgtatattgatgtcagtactacacttttaattactattgcttcagggtatgtcttgtaaaccaaaaataaaattataggccccccccgcccctgcacaaccaactgaatggacccatcctctcagccaagggcattccaaaattaacctgaaaaactagttcaagccatgatgggaagggggagttggacatgtctcatcacaccctactaccttttggaattactgatagaacagactcttaaagtctgaaaagaaacatttacaacctaccctctctgaagcctgctacctgggagcttcatctgcatgataaaaccttggtctccacaaccccttatggtaacccaaacattcctttctgttgataataactctttcaactagttgccaattagaaaatctttaaatcttcctatgacctagaaacctccctacccccactttgagttgtcctgcctttcctgacagaactcatgtacatcttacatatattgattgatgcctcatgtctccctaaaatgtataaaacaaagctgtaccccaccaccttggggacatgtcatcaggacctcctgtggctgtgtcataggagcgtctttaactttggcaaaataaactttctaaattgattgaaacctgtcttagctacttctggtttacagtcttaaagttagataatgtaaattgtccagctttggtttatttttgtccttagtagttccatataaattttagaatcagcttttcaatttaatacactactttcctcttagatccacaattaaatatatttgatgctaacaattctgttttatgtttttcgttttttttttttgagacaagagtttcgctcttgttgcccaggctggagtgcagtggcgcgatcttggctcaccacaacctccacctcccaggttcaagcaattcttctgcctcagcctcccgagtagctgggattacaggcatgcgccaccacgcccggctaattttgtatttttagtagagacggggtttcaccatgttgatcaggctggtcttgaactcctgacctcaggtgatccacccacctcggcctcccaaagtgttgggattacaggcgtgaaccaccatgcctggccagttctgttatttttaaaacccaagtttccctggtcatatcttggttggatgaagcgtattttcaatagattaccctggaaaggctagtgagtacggtattcttctacattttagacttttcttagtcttgctacttcaaggacagctaggctgcatataaaattcttggctcatactttttccccataaatttctatgagaaagtctaatgataactgattttctttattttgtaacttagtctttttgcttagaggctctctgaggatgggagggggttcttcctcccatccctaggaatttttcttttttttaaattcctaatcactagaccaccaggaagattgtttgttttgttttgtttttattcttcagggaccccatttatacatacgttaaataaatactgtttgccaatgtatcaaccattttgcttcttatttatttttgttcctttggttctttttcatggctttgctttggtgctccttagattttcagtcagatgtatttgtccttgggtaccttgtaatcagtattaccttttcttctgtcgctttgttttctgttcgttttgaaattacttgtttcctggtctggcaataacagttgagatatgaggagtttgagctgccatctgtctatgtatcttgctttaagactgcactcttctattgatatcactggccttgattttgtgatttctttatttcttcaggaccacccttcattttctactgtttgcttcctttttttttgagatggagtctcactctgtcactcaggctggagtgcagtgatcttggctcattgcaacctctgcctcccgggttccagcaattctcctgcctcagcctcccaagtatctgggactacaggtgtgcaccaccatgcccggctaagttttgtatttttaatagagacggggttttgccacattggcaggctggtctcaaactcctgatgtcaagtgatccacccaccccacccacctctgcatcccaaagtgctgggattacaggaatgagctgccgtgcccagcctcccccctacccccctttttttctttcgagacagagattataggtgtgagccactggacccagcctgtttttattccttttaccaaatctccaaggaatatcttcccttccaagtgcgaatgtaaccttaagtcagttaacctctttgtgattacttttcttatctgcaaagtgacttaatgatcttaagtactttttttttttgagacagggtctcactgtcaccctggctggagtgcagtggcacgatctctgatctccactcactgcaatctcctcttccctggttcaagcggccctcccaccttagccttctgggtagctgggactacagatgtgaaccaccacgcccagctaatttttgtactttttgtagagatggggttttgccatgttgcccaggctgggattattaagtactttttatcatacagcaagattgacattttatattggaatacatttgtctctatataacggagattaacaggaaaatgacaagcctgggtgcggtggctcatgcctgtaatcccagcactttgggaggctgaggtgggaggatcacttgaggtcaggagttcgagaccagttttgccaagatgatgaaagcccatgtctactaaaaatacaaaaattagcccagcttgatggtgggcgcctataatcccagctatttgagagactgaggcaggagaatcacttgaacctgggcagcagaggttgcagtgagccgagatcatgccactgcactccagcctgggtggcatagcgagactcttgtctcaagagaaaacaaaacaaaacaaaaaaaaaacaggaaaatgacaaaaagtaatattacaactcagtgaattttataacaaacttttttggaattcattgactaatactataccaaatccaaaatactctctagtataccaaatccaactctaccctatagtataaattggattctatttggacttgtctcactaatccctcatacagtgtgttttattttttattgaagtaaaaaaatttgtcattttaaccatttttaagtatatagttcagtaatattaagtatgttcatgttgttgcgcaatagatcttcggaagtttttcgtcttgcaacctgaaactctacccattagcaaattcccatttctccttacacttagcccttggtaatcatcattcttttttttttttttttgagatggagttttactcttgttgcccaggctggagtgcaatggtgcaatctcgactcaccacaacctccgcctcccaggttcaagcaattctacctcagcctcccgagtagctgggattacagtcatgcaccaccacgcccggctaattttgtatttttagtagagaaggggtttctccatgttgaggctggtctcgaactcctgacctcaggtgatctgcccacctcggcctcccaaagtgctgggattacaggcgtgagccactgcgcctggcccattctttctaattctataaatttgactacttagttaccttacataaataaattcttatagttagtgttatttttgcttccatgccttttttgttgttgttcatgctcttacttggaatgcgttctattttgtctacctatgcacatcctgttgggttttttttttttttgggggttttttttgtttttttttgtttttttttcccagacaaggtctcaatttgttacccaggctggagtgcagcggcgccatctccactcactgcatcctcaacttcctgggcccaggtgatcctctcgcctcagcccctgcaggtagctgggactataggcatgtgccaccatgcccagctaaatttggtttttttgtttgtttgtttttgagacagagtctcactctgtcacccaggctggagtgcagtggcacaatctcagctcactgcaatctctgccgcccgggttcaagtgattctcctgcctcagcctcccaagcagctgggattacaggtgactgccaccacgccagctaagttttgtagttttagtagagatggggtttcaccttgttggccatgctggtctcgaactcctgacctcgtgatctgcctgcttctgcctcccaaagtgctggaattacaggcatgagccaccacgcccggccagaatttttgtatttttagtagacacaaggttcttaccctgttgcctaggctggtctggaagtcctggactcaagcaattcacctgccttggcctcccaaaatgctgggattacaagccaccatgcccggcctaaatcctgttgttttgttttgttttattttgttttgttttgttttgtttgttttttgagacagagtctcgctatgtctctcaggctgtagtgcagtggcgcgatcttggctcactgccacctctgcctcccaggttcaagtgattctcctgcctcagcctcccaagtagctgggattacaggcatgtgctactatgtccggctaatttttgtatttttagtagagacagggtttcaccatgttggccaggctggtctcgaactcctgacctcgtgatccacccacctcggccacccaaagtgctgggattacaggcgtgagtggtttttatttcttaggccggtttcctccatatgatcttgcagtagacattaatttctttcctttttaattaaaatactgtttgtatttcacattttgatgtttgttaagatttgttttatattgttttttgttttgtcttgtgtgatagtcttaaatccctagttagataataactggagagtaccatgtttctatatatctctcagtgacttgcacagtgctagcagatagtgctaaaaaattatttattattattattattttgttattgttgttgttgttgttagacagggtcttcctctgtcacccaggctagagggcaatgggatgatcatagcttactgcagcctccaacaactgggctcatgtaattctcctgcctcagcttcccaagtagctgggattacaggcatgagccaccatgtctggacaaaaatatttccaggtgcagtggctcatgcctgtaattcccacacttgggaggccgagcgaggctggaggatcacttgagcctaggagttcaagaccagcttggctaagatggcgagaccccgtccctacaaaaaattttaaaaactagccaggcatggtggcatgcacctatattcccaactactcagtgggctgaggtgggagggtcatttgaacacaggaatttgaggggagaaaaaaagaagagagaaagagaagtgaaggaaggaagaaaggaaggagggagggagagaagaaagaaacgaaagaaaggaaaagaaaaggaaggaaagaaaattggtaccaggaaagcaggaaagggaaatggaagtaaaaaaataataataataataaaatgaaaattggttagtcactattaacaatttgtatccttataatctggaaacattataatttcaaaagaaaaaatattctttggatcataggttctgaggtcagaacagcattcccgtagtctagatgaagtcaagttttatctgatcttaattgaaataaatatagctggccttgaacaaatctactcatggtatgtggataggaattaaattgtaggggcattcacttgatggcattcattcttagaacatttacctatgtctagcttttggagtaaagtcacataacctctaaccaggtaagtttcctgtggctttatttaggattttaaatactcattttcagtgtaattttgttatgtgtggattaagatgactcttggtactaacatacattttctgattaaacctatctgaacatgagttgtttttatttcttaccctttccagagcgatgattctgacatttgggatgatacagcactgataaaagcatatgataaagctgtggcttcatttaaggtatgaaatgcttgcttagtcgttttcttattttctcgttattcatttggaaaggaattgataacatacgataaagtgttaaagtacatgttattcagttttcattttgaagattagatggtagtatgagttagttaaatcaggtgatatcctcctttagaagttgatagcctatatatgtcatcctttgtggaggcaatttaaataaaatttaaaacatttattcctggctgggtatggtggctcactcctgtaatcccagcactttgagaggctgaggcgggtggatcacctgaggtcaggagtttgagaccagcctggccaacatggtgaaaccccgtctttactaaaaatacaaaaattagccaagcatggtggcacgtgcctgtaatcccagctgcttgggacactgaggcaggagaattgcttgaacctggggggcagaggttgcaatgattgcaccactgcactccagcctgggcgatagagtgagactccatctcagaaaacgaacaaacaatgtattccttttagtatttttacattgtatcaaactatggaagtcctctaattgagattaataagaaaaagacaatctgaattataattttaaacatttaacaagcatgtagtaaaataatgatgaagataaatagcattagtacagcaattaatatttgtagcatgctgacagtgctctgtgtgcgtttcatatattaaattactctaatcatcccaaatcctgtaagttgggtatcaattcaagtgttcctattgggtaggaatatacagttcttttaggaaatgtagtatggttctgtgtctcaaacaggacacttacacagttggccaacatcatcaccttctccattctctgagatgtttagtcttactgagcactaaatatgggtcatcaatagtccagactaccttgagcaaacaatagtccagactaccttgagcaaacagagcatatactcatacagtgtataaagagcaccaagcatacagatttcatgtctttctcatagttactcttgtaacatgagctaaagatcagacctctatgtcacctttgtaactgatttctagatttttttttttttttgagatggggtcttgccctgtcacccaggctggagtgtagtggcgtgatcatgcctcattggagccttcaactcatgagctcaaacaatcctcctacctcagcttcctgagtagttgggaccacaggtgtgtgccaccacacccagctcatttttgtattctttgtagagatgcagtctcaccctgttgcccacgctggcctggaactcctgagctcaaaagatccctccgccttgaccttccaaagtgctgggattacaagcatgaaccactgcacccggcctagatttttaaatgtgctttccagtatacactgaaactagaagtcgactaaagaattaccaagagaattctataaaatagagattgaaatggggctcgatgtgggatgggttggtgatattgcagggagaagtaatctgagtaaaggaggaaaagaactgatttgggaaaacgatagttttagtagtgagtttgagtatgaattaagttgagattgaatttgaattaagttgaggttgaatatgaattaagttgaggttgagtttgaggtatgaattaagatgtgaaattgatcattggaaatgttagattgagaaaagtcacagctggattaatagcttcagaagtgtgtttgcagacagttgcaactaaagtaataagaatagatggccttggccgggcgcggtggctcacgcctgtaatcccagtactttgggaggctgaggcgagcaaatcacgaggtcaggagttcaagaccagcctggcccacatggtgaaaccccgtctttattaaaaatacaaaaattagctgtgcacagtggtgcacgcctgtaatcccagctactcgggaggctgagacaggagaatcgcttgaacctgggaggtggaggttgcagtgagctgagatcagtgtgactgcactccagcccggtgacagagtgagactctgtgtaaaaaaataaaataaataaaataatggccgtaagcaagtaaagaaggatggccagctcttattgggaatgcctaaatctaaggcttgatcagaagtaatgaaaccgttggggccctacattgctatgacatccaaagggccatgaatatcaggaagaaagataattaacagggtctaatgttacagagaggttgagagcaaggagatttgattaaaagggtctttagagctgatgtcaggtgtatgatgcctttaagagcagtttttatagtgcagggggtggtcaaaagagaaaataggtgctttctgaggtgacggagccttgagactagcttatagtagtaactgggttatgtcgtgacttttattctgtgcaccaccctgtaacatgtacatttttattcctattttcgtagcatgctctaaagaatggtgacatttgtgaaacttcgggtaaaccaaaaaccacacctaaaagaaaacctgctaagaagaataaaagccaaaagaagaatactgcagcttccttacaacaggttattttaaaatgttgagatttaacttcaaaggatgtctcattagtccttatttaatagtgtaaaatgtctttaacttaagtgattagtacagtgtttctattgacatatacttatacaacttcaaaaacaactattaaattttctgttatttaggaacatgcatattagtcatgaaagtataaagaattagatgggaatgataaatgctaaaatcaggacatgtgttccatttgtgaatggaaggcagggagaaggtgccgtttggaaggagtacccaagagccgtaagctgaattggcagtgttttacatcttaagctgagagatagatttttttttcccctttttctttaaaaactctaaaactgttaattccaaggaacccagaagtctaggtagattatttctgctagttaaaagcagtagtcctgaaagctgaatattttggtgtcttttgagccaactttagtttcatcattaccaagggggaagagagctaacagttgatgagcacttgctctaggccagtccagagtgctgggcaccatacgcattttatctccctcccgctattcacaacaaatatgggaggtagtttatattatagccatctaataagatggggaaactaagactcaaagagattcagaaacttgtccatgattataaatgtaagagagttggaattcagatttatgtatttagaccccaagcctttctcattacatcattttgccttccaaatctctaccctctatccttcacctccccactgatcaaaacgagatgatagtttgccctcttcaaaagaaatgtgtgcatgtatatatctttgatttcttttgtagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagaggtaaggatacaaaaaaaaaaaaattcaatttctggaagcagagactagatgagaaactgttaaacagtatacacagttgtcagtttgatccaccgaggcattaattttttcttaatcacacccttataacaaaaacctgcatattttttctttttaaagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaaggtaaaccttctatgaaagttttccagaaaatagttaatgtcgggacatttaacctctctgttaactaatttgtagctctcccatgaaacttttgtagcttaaatacacaagaattttttgaaaaggaaataagataatgatgcaaaatagttaattttttaaaaaaatgttagacactgcagtggatgcaacaaaatactttatatgaaagatttatccagttaacttttgtggagtattaggtattagactaataattagcacacttacttaagttagaaagtataataatgcgccggacgcggtagctcacgcctgtaatcccagcactttgggaggccaaggtgggcggatcacaaggtcaggagatcgagaccatcctggctaacacggtgaaaccccatctctactgaaaatacaaaaaaatttgccgggcgtgatggcgggcacctgtagtcccagctactcgggaggctgaggcaggaggatggtgtgaaccccggaggcagagcttgcagtgagtcaagatcgtgccactgcactccaacctgggcgacagaatgagactccatctcaaacaaaaaaacaaaacaaaacaaaaaaaagtgtaataataatttatcattagctggatgatatgctgttgtttcccatgtcacctgtataagatatgtaaaataagaacacattatttacatctaatatagataaaatcctgaggcgctctcagattgttttgtagagttcaaatgtaaatattgttttcatttatggtccttttggttataagtaacagaaatcaactctaaaaagatttttattataggttagattatgtcatggaaccttaaggcttgtccctttctagttcttttgtgtaaagcggtgatttcttccatggagggaatggtatttaggcaatttttttttttttttcgagatggagtcttgctctgtcgctcaggctggagtgcagtggcaccatttcagctcactgcaacttccacctcctgggttcaagtgattctcctgcttcagcctcccaagtagctgagattacaggcacccgccaccacacccggcttattttgtatttttagtagagatggggtttcaccatgttggccaggctggtcttgaactcctgacctcaagtgatctccccaccttggccttccaaagtgctaggattacaggcgcctagcctaggcagtcattttcaaaaaacaagcatgactcaccaaaagttttaagattttctgtgataatgttcttattgaggcttacattatattacagtttcttgaatctaaaatgatgtaccctcttagaatatatacatcatgcttcattggtctcagggggctgatttttataaggagagatttgctagttttcacaatatgtcctctaagttggcatgtatagctaaacaggctttcataaaaatatacaatttagttaatgaaatttgggatatagtcttttatgattgaaataattttgctaaatagactgtctctgatttattaggtaatcaccactcttattttgttttacttccttaatgtctacatagaaaggaaatgagaaaaatccagaggttgtcatttgacttatgagtctgtttgacttcaggatttggtacatgaaatttcacttaatctttttgatatgtataaaacaaatattctgggtaattatttttatccttttggttttgagtcctttttattcctatcatattgaaattggtaagttaattttcctttgaaatattccttatagccaggtctaaaattcaatggcccaccaccgccaccgccaccaccaccaccccacttactatcatgctggctgcctccatttccttctggaccaccagtaagtaaaaaagagtataggttagattttgctttcacatacaatttgataattagcagaatagaggattgtaaaatgtcattgtagaacatcccttgggccagattctaatgggtagaaatttgaactaaacctctgggttttgtttgtttttaatgcctttctgttacccagatgcagtgctcttgtagtcccaagtctaagctctaggttgccttctttcctggcagaagttggtgtctatgccataaggaggtagttcctgttagaagggatttaattataccttatataaggaattagtgtttgcccttctaggtatagttggatgttagcttctgatgtaaactggatttctttttctttctctctctttttttttttttgttttggaggcagagttttgcccttgtaccccaggctggagtgcagtggtgtgatctcagctcacagcaacctccgcctcctgggttcaagcaattctgcctcggcctcccaagtagctgggattacaggcgactgccaccacacccggctaatttttgttttattagtagagatggggtttcaccatgttggccagactgatcttgaactcctgacctcaggtgatccacccgccttggcctcccaaagcgctgggattacaggcgtgagctgccgcacccagctgtaaactggatttctaatggtagatttttaggtattaacaatagataaaaagatactttttggcatactgtgtattgggatggggttagaacaggtgttctacccaagacatttacttaaaatcgccctcgaaatgctatgtgagctgtgtgtgtgtgtgtgtgtgtgtgtgtattaaggaaaagcatgaaagtatttatgcttgattttttttttttactcatagcttcatagtggaacagatacatagtctaaatcaaaatgtttaaactttttatgtcacttgctgtcttttcgtcctcgttaaatttaattttgttggtcttttgttgttattggttggttttctccaaatgctagctatgttaagaaatttaaggccaggtacagtggctcatgcctgtaatcccggcattttagaaggctgaggcaggaggatcacttgagctcaggagtttgagaccagtctgggcaacatagcaagacctcgtctttgtttaggggaaaaaaaagaaatttaagtaggagattatataagcaaaaatacaattaatttccagcattcactatataatataaatctccagactttacttttttgtttactggatataaacaatatctttttctgtctccagataattcccccaccacctcccatatgtccagattctcttgatgatgctgatgctttgggaagtatgttaatttcatggtacatgagtggctatcatactggctattatatggtaagtaatcactcagcatcttttcctgacaatttttttgtagttatgtgactttgttttgtaaatttataaaatactacttgcttctctctttatattactaaaaaataaaaataaaaaaatacaactgtctgaggcttaaattactcttgcattgtccctaagtataattttagttaattttaaaaagctttcatgctattgttagattattttgattatacacttttgaattgaaattatactttttctaaataatgttttaatctctgatttgaaattgattgtagggaatggaaaagatgggataatttttcataaatgaaaaatgaaattcttttttttttttttttttttttgagacggagtcttgctctgttgcccaggctggagtgcaatggcgtgatcttggctcacagcaagctctgcctcctggattcacgccattctcctgcctcagcctcagaggtagctgggactacaggtgcctgccaccacgcctgtctaattttttgtatttttttgtaaagacagggtttcactgtgttagccaggatggtctcaatctcctgaccccgtgatccacccgcctcggccttccaagagaaatgaaatttttttaatgcacaaagatctggggtaatgtgtaccacattgaaccttggggagtatggcttcaaacttgtcactttatacgttagtctcctacggacatgttctattgtattttagtcagaacatttaaaattattttattttattttattttttttttttttttgagacggagtctcgctctgtcacccaggctggagtacagtggcgcagtctcggctcactgcaagctccgcctcccgggttcacgccattctcctgcctcagcctctccgagtagctgggactacaggcgcccgccaccacgcccggctaatttttttttatttttagtagagacggggtttcaccgtggtctcgatctcctgacctcgtgatccacccgcctcggcctcccaaagtgctgggattacaagcgtgagccaccgcgcccggcctaaaattatttttaaaagtaagctcttgtgccctgctaaaattatgatgtgatattgtaggcacttgtatttttagtaaattaatatagaagaaacaactgacttaaaggtgtatgtttttaaatgtatcatctgtgtgtgcccccattaatattcttatttaaaagttaaggccagacatggtggcttacaactgtaatcccaacagtttgtgaggccgaggcaggcagatcacttgaggtcaggagtttgagaccagcctggccaacatgatgaaaccttgtctctactaaaaataccaaaaaaaatttagccaggcatggtggcacatgcctgtaatccgagctacttgggaggctgtggcaggaaaattgctttaatctgggaggcagaggttgcagtgagttgagattgtgccactgcactccacccttggtgacagagtgagattccatctcaaaaaaagaaaaaggcctggcacggtggctcacacctataatcccagtactttgggaggtagaggcaggtggatcacttgaggttaggagttcaggaccagcctggccaacatggtgactactccatttctactaaatacacaaaacttagcccagtggcgggcagttgtaatcccagctacttgagaggttgaggcaggagaatcacttgaacctgggaggcagaggttgcagtgagccgagatcacaccgctgcactctagcctggccaacagagtgagaatttgcggagggaaaaaaaagtcacgcttcagttgttgtagtataaccttggtatattgtatgtatcatgaattcctcattttaatgaccaaaaagtaataaatcaacagcttgtaatttgttttgagatcagttatctgactgtaacactgtaggcttttgtgttttttaaattatgaaatatttgaaaaaaatacataatgtatatataaagtattggtataatttatgttctaaataactttcttgagaaataattcacatggtgtgcagtttacctttgaaagtatacaagttggctgggcacaatggctcacgcctgtaatcccagcactttgggaggccagggcaggtggatcacgaggtcaggagatcgagaccatcctggctaacatggtgaaaccccgtctctactaaaagtacaaaaacaaattagccgggcatgttggcgggcaccttttgtcccagctgctcgggaggctgaggcaggagagtggcgtgaacccaggaggtggagcttgcagtgagccgagattgtgccagtgcactccagcctgggcgacagagcgagactctgtctcaaaaaataaaataaaaaagaaagtatacaagtcagtggttttggttttcagttatgcaaccatcactacaatttaagaacattttcatcaccccaaaaagaaaccctgttaccttcattttccccagccctaggcagtcagtacactttctgtctctatgaatttgtctattttagatattatatataaacggaattatacgatatgtggtcttttgtgtctggcttctttcacttagcatgctattttcaagattcatccatgctgtagaatgcaccagtactgcattccttcttattgctgaatattctgttgtttggttatatcacattttatccattcatcagttcatggacatttaggttgtttttatttttgggctataatgaataatgttgctatgaacattcgtttgtgttctttttgtttttttggttttttgggttttttttgttttgtttttgtttttgagacagtcttgctctgtctcctaagctggagtgcagtggcatgatcttggcttactgcaagctctgcctcccgggttcacaccattctcctgcctcagcccgacaagtagctgggactacaggcgtgtgccaccatgcacggctaattttttgtatttttagtagagatggggtttcaccgtgttagccaggatggtctcgatctcctgacctcgtgatctgcctgcctaggcctcccaaagtgctgggattacaggcgtgagccactgcacctggccttaagtgtttttaatacgtcattgccttaagctaacaattcttaacctttgttctactgaagccacgtggttgagataggctctgagtctagcttttaacctctatctttttgtcttagaaatctaagcagaatgcaaatgactaagaataatgttgttgaaataacataaaataggttataactttgatactcattagtaacaaatctttcaatacatcttacggtctgttaggtgtagattagtaatgaagtgggaagccactgcaagctagtatacatgtagggaaagatagaaagcattgaagccagaagagagacagaggacatttgggctagatctgacaagaaaaacaaatgttttagtattaatttttgactttaaattttttttttatttagtgaatactggtgtttaatggtctcattttaataagtatgacacaggtagtttaaggtcatatattttatttgatgaaaataaggtataggccgggcacggtggctcacacctgtaatcccagcactttgggaggccgaggcaggcggatcacctgaggtcgggagttagagactagcctcaacatggagaaaccccgtctctactaaaaaaaatacaaaattaggcgggcgtggtggtgcatgcctgtaatcccagctactcaggaggctgaggcaggagaattgcttgaacctgggaggtggaggttgcggtgagccgagatcacctcattgcactccagcctgggcaacaagagcaaaactccatctcaaaaaaaaaaaaataaggtataagcgggctcaggaacatcattggacatactgaaagaagaaaaatcagctgggcgcagtggctcacgccggtaatcccaacactttgggaggccaaggcaggcgaatcacctgaagtcgggagttccagatcagcctgaccaacatggagaaaccctgtctctactaaaaatacaaaactagccgggcatggtggcgcatgcctgtaatcccagctacttgggaggctgaggcaggagaattgcttgaaccgagaaggcggaggttgcggtgagccaagattgcaccattgcactccagcctgggcaacaagagcgaaactccgtctcaaaaaaaaaaggaagaaaaatatttttttaaattaattagtttatttattttttaagatggagttttgccctgtcacccaggctggggtgcaatggtgcaatctcggctcactgcaacctccgcctcctgggttcaagtgattctcctgcctcagcttcccgagtagctgtgattacagccatatgccaccacgcccagccagttttgtgttttgttttgttttttgttttttttttttgagagggtgtcttgctctgtcccccaagctggagtgcagcggcgcgatcttggctcactgcaagctctgcctcccaggttcacaccattctcttgcctcagcctcccgagtagctgggactacaggtgcccgccaccacacccggctaatttttttgtgtttttagtagagatggggtttcactgtgttagccaggatggtctcgatctcctgaccttttgatccacccgcctcagcctccccaagtgctgggattataggcgtgagccactgtgcccggcctagtcttgtatttttagtagagtcgggatttctccatgttggtcaggctgttctccaaatccgacctcaggtgatccgcccgccttggcctccaaaagtgcaaggcaaggcattacaggcatgagccactgtgaccggcaatgtttttaaattttttacatttaaattttattttttagagaccaggtctcactctattgctcaggctggagtgcaagggcacattcacagctcactgcagccttgacctccagggctcaagcagtcctctcacctcagtttcccgagtagctgggactacagtgataatgccactgcacctggctaatttttatttttatttatttatttttttttgagacagagtcttgctctgtcacccaggctggagtgcagtggtgtaaatctcagctcactgcagcctccgcctcctgggttcaagtgattctcctgcctcaacctcccaagtagctgggattagaggtccccaccaccatgcctggctaattttttgtactttcagtagaaacggggttttgccatgttggccaggctgttctcgaactcctgagctcaggtgatccaactgtctcggcctcccaaagtgctgggattacaggcgtgagccactgtgcctagcctgagccaccacgccggcctaatttttaaattttttgtagagacagggtctcattatgttgcccagggtggtgtcaagctccaggtctcaagtgatccccctacctccgcctcccaaagttgtgggattgtaggcatgagccactgcaagaaaaccttaactgcagcctaataattgttttctttgggataacttttaaagtacattaaaagactatcaacttaatttctgatcatattttgttgaataaaataagtaaaatgtcttgtgaaacaaaatgctttttaacatccatataaagctatctatatatagctatctatatctatatagctattttttttaacttcctttattttccttacagggttttagacaaaatcaaaaagaaggaaggtgctcacattccttaaattaaggagtaagtctgccagcattatgaaagtgaatcttacttttgtaaaactttatggtttgtggaaaacaaatgtttttgaacatttaaaaagttcagatgttagaaagttgaaaggttaatgtaaaacaatcaatattaaagaattttgatgccaaaactattagataaaaggttaatctacatccctactagaattctcatacttaactggttggttgtgtggaagaaacatactttcacaataaagagctttaggatatgatgccattttatatcactagtaggcagaccagcagacttttttttattgtgatatgggataacctaggcatactgcactgtacactctgacatatgaagtgctctagtcaagtttaactggtgtccacagaggacatggtttaactggaattcgtcaagcctctggttctaatttctcatttgcaggaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactctattttgaaaaaccatctgtaaaagactgaggtgggggtgggaggccagcacggtggtgaggcagttgagaaaatttgaatgtggattagattttgaatgatattggataattattggtaattttatgagctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtatataataaaaatatttaatttttttttaaattagctgtatctgtgattgtatttcttttttgcatattattttgccctttggcccatattttgatatggatgccaccatagcattttgtgtatgtgcatgtgtattcccacttaatgtcacatttttcatgtctttacatattcttatttttgtttgtttttgagacagagtctcgctctgctgcccacgctggagtgcagtggtgcaatctcagctcactgcaacctctgctatccgggttcaagcagttctcgtgcctcacccacgtgagtagttgggattacaggcatgtggcaccatgccccactaagttttgtatttttagtagagatggagtttcaccatgttggccaggctggtctcaaactcctgccctcaagtgattcgaccaccctggcctcccaaagtgctgggattacagccgtgagccaccgcacacggcctctctatttatttctatacatagcttttcacattatattatgtttatatattgtttatatctgtatttcctctttcattagagaaaaggtagtacatcttattcttcatggtgtctacaatatctggcagtttttggaagtcaagcgtgagcttagagcatagactggtgggattgtcaaagaagagggcaactggaagagaactgtcagttatttttggatcagtctttaattcatcatgacgggttaggcattagttgtatttcttgctaattttgaagaagacttattaacaaatcctacattaggtaaatggttttgaaagttgagttaatcataatggtgtttgacctaggactatttttaggccctatttatcttaatatcgaataatgaagcagcttcccccttagatatagacagaaaacatcaaagccaccacactacctggctggatttatcctagtaataaaatcaaaactgagctagttctctggctttcattgtaataattgtccttgtggttgtaaggaatctagatgaaaattacatggtctgttctacagccacagctgtacctacattcagaagacagacaaaagttgctgtgtttgaagagatccttcattaagggatcagacagagattactttgagacatattctaagtttaacttttctgcagggttgccattaacagaaataaactacagagttaatttctttttgtttttgatacagtctaactctcacccaagctggagtgcagtggcgcaatttcagctcactgcaacctctgcctcccaggttcaagcaattctcctgcctcagcctcccgagcagctgggactacaggcatgtgccactatgcctggctaatttttgtatttttagtagtagagacgtggtttcgccacgttggccaggctggtctggaactcctgaccccaggtaatccacctgcctcggcctcccaaagtgctgggattacaagcttgagccactacgcctgacccagagttaactttttaaaaaagtttttatgaacttaagtcttgtgatgtttgaaataatggattcaatttagacatcaaattccagaagttactaagagcagctgggcgcggcagctcacacctgtaatcccagcactttgggaggccgaggcgggtggatcacctgagatcaggagttccagaccagcctggccaacatagtaaaaccctgtctctactaaaaatacaaaaattagcccggcatggtggcacgccctgtagtcccagctacttgggaggctgaggcaggagaattgcttgaacccgggaggtggaggttgtggtgagccgagattgtgccactgtactcaagcctgggctaaaaagcgagactccgtctcaaaaaaaaaaaaaaaaaaaacacgttactaagagcaactctgggccaggcacggtggcttacacctgtaatcccagcattttgggaggacgagacaggcggatcacttgagcccaggagttcaagaccagcataagcaacaacgcaaaacccctgactctacaa

(19) United States
(12) Patent Application Publication
(10) Pub. No.: US 2014/0343127 A1
(43) Pub. Date: Nov. 20, 2014

(54) COMPOUNDS FOR THE MODULATION OF SMN2 SPLICING

(71) Applicant: Santaris Pharma A/S, Horsholm (DK)

(72) Inventor: Susanne Kammler, Holte (DK)

(73) Assignee: Santaris Pharma A/S a corporation

(21) Appl. No.: 14/357,385

(22) PCT Filed: Nov. 8, 2012
(86) PCT No.: PCT/EP2012/072100

§ 371 Date: May 9, 2014

Related U.S. Application Data
(60) Provisional application No. 61/558,462, filed on Nov. 11, 2011.

(51) Int. Cl. C12N 15/113

(52) U.S. Cl. CPC C12N 15/113
USPC 514/44 A; 536/24.1; 435/366

(57) ABSTRACT

The present invention relates to oligomer compounds (oligomers) which target nucleic acids encoding human SMN2 in a cell, leading to modulation of SMN2 mRNA splicing which favors full length SMN2 mRNA rather than the poorly functional truncated transcript, SMN2 Δ7. Reduction of SMNA7 mRNA expression and/or increase in full length SMN2 mRNA expression are beneficial for the treatment of diseases or disorders associated with overexpression or undesirably high levels of aberrant forms of SMN2, particularly SMN2 Δ7, such as spinal muscular atrophy (SMA).

### FIELD OF INVENTION

The present invention relates to oligomeric compounds (oligomers) that target survival of motor neuron 2 (SMN2) RNA, leading to a modulation of SMN2 mRNA splicing. Modulation of SMN2 splicing is believed to be beneficial for treatment of spinal muscular atrophy (SMA).

### BACKGROUND

Spinal muscular atrophy (SMA) is an autosomal recessive genetic neuromuscular disease characterized by degeneration of motor neurons in the spinal cord, causing progressive weakness of the limbs and trunk, followed by muscle atrophy and death by respiratory failure. SMA is the most common genetic cause of death in early childhood. SMA patients are generally classified into types I-III based on age at onset and clinical course. However, all three types of SMA are caused by mutations in the survival motor neuron gene (SMN1); 96% of SMA patients display mutations in this gene. Wirth, B. (2000), Human Mutation, 15: 228-237 There are two near-identical copies of this gene, SMN1 and SMN2, at the same chromosomal locus, 5q13. Homozygous loss-of-function mutation or deletion of SMN1 is responsible for SMA; in contrast, homozygous absence of SMN2 has no clinical phenotype and is found in about 5% of healthy controls. The presence of SMN2 does not necessarily mitigate the effects of SMN1 absence because a single nucleotide difference between SMN1 and SMN2 causes skipping of SMN2 exon 7 and production of a largely nonfunctional protein referred to as SMNΔ7. SMA disease severity is inversely proportional to the number of genomic copies of the SMN2 gene present.

A major goal in SMA research has been to improve expression of functional SMN protein from SMN2. Increasing SMN2 exon 7 inclusion by modulation of splicing has been studied intensely as a means to elevate full-length SMN protein levels in SMA.

Signals located within an exon can have positive or negative effects on the recognition of that exon during splicing. Exonic splicing enhancers (ESEs) stimulate splicing and are often required for efficient intron removal, whereas exonic splicing silencers (ESSs) inhibit splicing. The single nucleotide difference between SMN2 and SMN1 is widely accepted as a major cause for SMN2 exon 7 skipping, probably by destroying an Exonic Splicing Enhancer (ESE) and/or turning it into an Exonic Splicing Silencer (ESS) binding hnRNP A1 instead [Kashima et al., (2003) Nature Gen 34:460-463; Cartegni et al., (2006) Am J Hum Genet. January; 78(1): 63-77; Hua et al. (2007) PLoS 5(4):e73].

Additionally several cis-acting splicing regulatory elements have been mapped in exon 7 and its surrounding intronic sequences (summarized in FIG. 1). In intron 6 there are 2 published silencer sequences named ISS-E1 [Miyajima et al., (2002) J. Biol. Chem. 277:23271-23277) and an unnamed silencer close to the 3′ss of intron 6 (Hua et al., (2008) Am J Hum Genet. 82:834-848].

Another enhancer in exon 7 (Tra2β binding site) is also crucial for exon 7 inclusion. A terminal stem loop structure (TSL-2) in exon 7 competes with U1snRNP recruitment to the 5′ss of intron 7 [Singh et al., (2006) Nucl. Acids Res. 35:371-389; Hua et al., 2007] and thereby enhances exon 7 skipping. In intron 7 a splicing silencer ISS-N1 enhances exon 7 skipping and was characterized as a tandem hnRNPA1/A2 motif (Singh 2006; Hua 2008). A second motif, ISE-E2, was first described as an enhancer for exon 7 splicing [Miyajima et al., (2003) J. Biol. Chem. 278:15825-15831] but later on an hnRNP A1 binding site was mapped close by. The binding site is generated by an A→G transition between SMN1 and SMN2 and indicates a bifunctional character of this element [Kashima et al., (2007) Proc. Natl. Acad. Sci. 104:3426-3431].

Because SMN protein itself functions in the pre-mRNA splicing pathway, it has been proposed that this protein may influence splicing of its own pre-mRNA. Jodelka et al. have shown that the abundance of SMN protein determines, in part, the outcome of SMN2 alternative splicing. Their results identify a feedback loop in SMN expression by which low SMN protein levels exacerbate SMN2 exon 7 skipping, leading to a further reduction in SMN protein. These results led the authors to suggest that a modest increase in SMN protein abundance may cause a disproportionately large increase in SMN expression and thus an significant likelihood of therapeutic effect. Jodelka, F. M. et al. Hum Mol Genet. 2010 December 15; 19(24): 4906-4917.

Several efforts have been made to modulate SMN2 splicing using oligonucleotides in in vitro experiments as well as in vivo mouse models. There are patent applications describing extensive targeting of specifically modified 2′-methoxyethoxy phosphorothiate oligonucleotides to sequences in exon 7 and sixty nucleotides upstream and downstream of the exon. This includes published regions ISS (intron 6), ESE/ISS and TSL2 in exon 7 and ISS-N1 in intron 7 (ISIS & Krainer et al., patent WO/2007/002390 A2; Hua et al., 2008). The resulting lead oligonucleotide, an 18-mer uniform 2′-MOE oligomer with a phosphorothioate backbone, targets ISS-N1, and was further investigated and taken into mouse models (WO/2010/120820 A1, WO/2010/148249 A1). and into cynomolgus monkeys in which it was shown that a single intraventricular injection delivered putative therapeutic levels of the oligonucleotide to all regions of the spinal cord. Passini et al. (2011) 3:72ra18. Singh et al. used a focused approach targeting the ISS-N1 region [US20070292408, Singh et al., (2009) RNA Biol. 6:341-350. In particular, a short 8mer 2′-O-methyl phosphorothioate oligonucleotide was described which targeted ISS-N1 and efficiently increased exon 7 inclusion.

Furthermore there are in vitro data using a single 2′-O-methyl phosphorothioate oligonucleotide, targeting ISS-E1 (intron 6) and a single 2′-O-methyl phosphorothioate oligonucleotide targeting ISE/ISS-E2 (intron 7). The first (“oligo-element 1”, Miyajima 2002) was found to increase exon 7 inclusion and the second, targeted to “element 2” in intron 7, was shown to decrease exon 7 inclusion, in contrast to the observations herein [Miyaso et al. (2003) J. Biol. Chem. 278:15825-15831]. Baughan et al. used a bifunctional 2′-0 methyl oligonucleotide to recruit splice supporting SR-proteins to the ISS-E1 element in intron 6 [Baughan et al. (2009) Hum Mol Ther. 18:1600-1611].

In spite of extensive efforts, no antisense compound has emerged as a treatment for SMA. The LNA oligomers of the instant invention are believed to be particularly well suited to splice switching and are thus believed to have therapeutic use in modulating SMN2 splicing, thus ameliorating the symptoms of this genetic condition.

### SUMMARY OF THE INVENTION

Herein are provided oligomers from 10 to 30 nucleotides in length which comprise at least one Locked Nucleic Acid (LNA) unit, and further comprise a nucleobase sequence of from 10 to 30 nucleobases in length, wherein said nucleobase sequence is at least 80% complementary to a region corresponding to nucleotides 26231-26300, 31881-31945, or 32111-32170 of Genbank Accession No. NG_{—}008728 (SEQ ID NO: 167) or a naturally occurring variant thereof.

The oligomers may modulate splicing of SMN2 mRNA resulting in an increase in levels of the full length mRNA transcript. The oligomers may be oligomers which do not elicit RNAse H cleavage

In some embodiments, the oligomer sequence is at least 80% complementary to a region corresponding to nucleotides 26231-26246, 26274-26300, 31890-31905, 31918-31945 or 32115-32162 of Genbank Accession No. NG_{—}008728 (SEQ ID NO: 167). In other embodiments, the oligomer sequence is at least 80% complementary to nucleotides 26231-26300 of Genbank Accession No. NG_{—}008728 (SEQ ID NO: 167). The oligomer may have a nucleobase sequence at least 80% identical to the sequence of SEQ ID NO: 1, 2, 3-16, 19-20, 22, 24-34, 35-38, 40, 41, 45-49, 60-80 or 83, and may have SEQ ID NO: 1, 5, 9, 11, 12, 26, 27, 28, 29, 30, 34, 40, 53-59, 62, 63, 65, 66, 69-77 or 79.

In some embodiments, the oligomer modulates splicing to increase the amount of the full length SMN2 transcript to greater than 110% of control, greater than 120% of control, greater than 130% of control, greater than 140% of control, greater than 150% of control greater than 160% of control, greater than 170% of control, greater than 180% of control, greater than 190% of control, or greater than 200% of control. The oligomer may be from 12 to 16 nucleotides in length and may be a mixmer.

Also provided is a conjugate comprising the foregoing oligomer and at least one non-nucleotide or non-polynucleotide moiety covalently attached to said oligomer. The oligomer or the conjugate may be used as a medicament, such as for the treatment of spinal muscular atrophy, including Type I, II and III spinal muscular atrophy.

Further provided is a pharmaceutical composition comprising the foregoing oligomer or the conjugate, and a pharmaceutically acceptable diluent, carrier, salt or adjuvant. Also provided is a method of treating spinal muscular atrophy, said method comprising administering an effective amount of the foregoing oligomer, conjugate, or pharmaceutical composition to a patient suffering from or believed likely to suffer from spinal muscular atrophy.

A method for modulating splicing of SMN2 mRNA in a human cell expressing SMN2 mRNA is also provided, using the oligomers, conjugates or pharmaceutical compositions provided herein. For example, the method may be in vivo or in vitro.

### BRIEF DESCRIPTION OF FIGURES

FIG. 1 is a schematic line drawing showing exons 6, 7 and 8 of the human SMN2 gene (gray boxes), introns 6 and 7 (located between exons 6 and 7 and exons 7 and 8, respectively), 5′ and 3′ splice sites (ss) and a series of splicing regulatory sequences (ISS, ESE/ESS, ISE/ISS, etc.) on the target sequence.

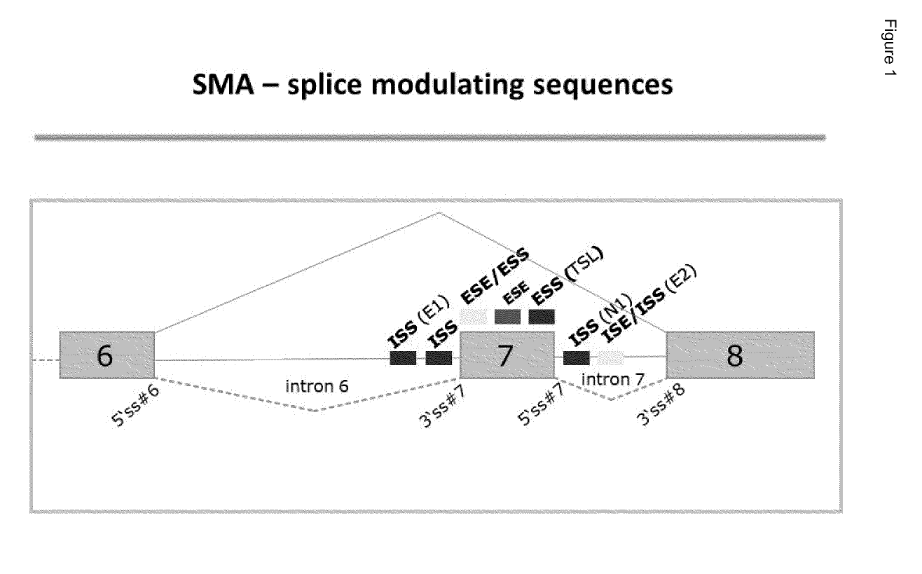

### DETAILED DESCRIPTION OF THE INVENTION

### The Oligomer

The present invention employs oligomeric compounds (referred herein as oligomers), for use in modulating the function of nucleic acid molecules encoding human SMN2, such as the SMN2 nucleic acid of Genbank Accession No. NG_{—}008728 and naturally occurring variants of such nucleic acid molecules encoding human SMN2. Genbank Accession No. NG_{—}00828 is a genomic nucleic acid sequence that encodes human SMN2 transcript variant d, which includes all exons.

The term “oligomer” in the context of the present invention, refers to a molecule formed by covalent linkage of two or more nucleotides (i.e. an oligonucleotide). Herein, a single nucleotide (unit) may also be referred to as a monomer or unit. In some embodiments, the terms “nucleoside”, “nucleotide”, “unit” and “monomer” are used interchangeably. It will be recognised that when referring to a sequence of nucleotides or monomers, what is referred to as the sequence of bases, such as A, T, G, C or U.

The oligomer consists or comprises of a contiguous nucleotide sequence of from 10-50 nucleotides in length, such as 10-30 nucleotides in length.

In various embodiments, the compound of the invention does not comprise RNA (units). It is preferred that the compound according to the invention is a linear molecule or is synthesised as a linear molecule. The oligomer is a single stranded molecule, and preferably does not comprise short regions of, for example, at least 3, 4 or 5 contiguous nucleotides, which are complementary to equivalent regions within the same oligomer (i.e. able to form duplexes). In some embodiments, the oligomer is essentially not double stranded, i.e., is not a siRNA. In various embodiments, the oligomer of the invention may consist entirely of the contiguous nucleotide region. Thus, the oligomer is not substantially self-complementary.

### The Target

Suitably the oligomer of the invention is capable of modulating splicing of human SMN2 mRNA. In this regard, the oligomer of the invention can affect aberrant splicing of SMN2, typically in a human cell. As will be understood, “aberrant” means excessive, unwanted or inappropriate.

The oligomers of the invention bind to the SMN2 nucleic acid and increase the levels of full length SMN2 mRNA compared to controls (e.g., untreated or mock treated controls) (i.e., to greater than 100% of control levels), and more preferably increase the levels of full length SMN2 RNA to at least 130%, 140%, 150%, 160%, 170%, 180%, 190% or 200% or more compared to the normal expression level (such as the expression level in the absence of the oligomer(s) or conjugate(s)). Preferably levels of full length SMN2 mRNA are increased to at least 150%, more preferably 200%, of control, i.e., intron 7 inclusion is increased. In some embodiments, the level of SMN2 Δ7 mRNA is decreased (exon 7 exclusion is decreased) as the level of full length SMN2 mRNA is increased. In other embodiments, both full length SMN2 and SMN2 Δ7 mRNA are increased.

In some embodiments the oligomers of the invention is administrated to a mammal, preferably a human in need for a modulation of SMN2 mRNA splicing. The oligomer dosage may be, for example be, between about 0.1 and about 100 mg/kg body weight such as between 0.1 and 1 mg/kg body weight per day, or between 1.0 and about 10 mg/kg body weight per day. Thus, for administration to a 70 kg person, in some embodiments, the dosage range may be about 7 mg to 0.7 g per day. In some embodiments each dose of the oligomer may, for example, be between about 0.1 mgs/kg or 1 mg/kg and about 10 mg/kg of 20 mg/kg, (i.e. a range of between e.g. 0.1 and 20 mg/kg, such as between 1 mg/kg and 12 mg/kg). Individual doses may therefore be, e.g. about 0.2 mg/kg, such as about 0.3 mg/kg, such as about 0.4 mg/kg, such as about 0.5 mg/kg, such as about 0.6 mg/kg, such as about 0.7 mg/kg, such as about 0.8 mg/kg, such as about 0.9 mg/kg, such as about 1 mg/kg, such as about 2 mg/kg, such as about 3 mg/kg, such as about 4 mg/kg, such as about 5 mg/kg, such as about 6 mg/kg, such as about 7 mg/kg, such as about 8 mgs/kg, such as about 9 mg/kg, such as about 10 mg/kg. In some embodiments the dose of the oligomer is below 7 mg/kg, such as below 5 mg/kg or below 3 mg/kg. In some embodiments the dose of the oligomer is above 0.5 mg/kg, such as above 1 mg/kg. In some embodiments, the time interval between each administration of the oligomer may be for example, selected from the group consisting of 1 day, 2 days, 3 days, 4 days, 5 days, 6 days and weekly. In some embodiments the time interval between administration is at least every other day, such as at least every three days, such as at least every 4 days, such as at least every 5 days, such as at least every 6 days, such as weekly, such as at least every two weeks (biweekly) or at least every 3 or 4 weeks, or at least monthly.

In some embodiments, such modulation is seen when using from 0.04 to 25 nM, such as from 0.8 to 20 nM, of the compound of the invention, e.g., 0.5, 1, 5, 20 or 25 nM. In other embodiments, such modulation is seen when using from 5 to 25 μM, such as from 8 to 20 μM, of the compound of the invention, e.g., 1, 5, 20 μM or 25 μM. Modulation of splicing of full length SMN2 may be determined by measuring SMN protein levels, e.g. by methods such as SDS-PAGE followed by western blotting using suitable antibodies raised against the appropriate regions of the target protein. Alternatively, modulation of splicing can be determined by measuring levels of mRNA, e.g. by northern blotting or quantitative RT-PCR using appropriate probes, such as for full length and/or Δ7 mRNA.

As illustrated herein the cell type may, in some embodiments, be a cell derived from a human patient with SMA, such as an SMA fibroblast cell line such as GM03813, Cornell Institute for Medical Research, Camden N.J.). The oligomer concentration used may, in some embodiments, be 5 nM. The oligomer concentration used may, in some embodiments, be 25 nM. The oligomer concentration used may, in some embodiments be 0.5 nM or 1 nM. This concentration of oligomer is typically used in an in vitro cell assay, using transfection (Lipofection), as illustrated in the examples. In the absence of a transfection agent, the oligo concentration required to obtain the down-regulation of the target is typically between 1 and 25 μM, such as 5 μM.

The invention therefore provides a method of modulating the splicing of SMN2 mRNA in a cell which is expressing SMN2 mRNA, said method comprising administering the oligomer or conjugate according to the invention to said cell to modulate the splicing of SMN2 mRNA in said cell. Suitably the cell is a human cell, such as a cell from an SMA patient. The administration may occur, in some embodiments, in vitro. The administration may occur, in some embodiments, in vivo.

The term “target nucleic acid”, as used herein refers to the DNA or RNA encoding a human SMN polypeptide, such as Genbank Accession No. NG_{—}008728 or naturally occurring variants thereof, and RNA nucleic acids derived therefrom, preferably RNA, including pre-mRNA and mature mRNA. In some embodiments, for example when used in research or diagnostics the “target nucleic acid” may be a cDNA or a synthetic oligonucleotide derived from the above DNA or RNA nucleic acid targets. The oligomer according to the invention is capable of hybridising to the target nucleic acid. It will be recognised that Genbank Acc. No. NG_{—}008728 is a genomic DNA sequence, and as such, corresponds to the pre-mRNA target sequences, although uracil is replaced with thymidine in the cDNA sequences. Targeting of the pre-mRNA is preferred for modulation of splicing. It will be understood that “targeting the mRNA” and “targeting the RNA” in the context of modulation of splicing are intended to mean “targeting the pre-mRNA”. “SMN2 splicing” will be understood to mean the maturation process in which the introns are spliced out of SMN2 pre-mRNA to yield a mature SMN2 mRNA.

The term “naturally occurring variant thereof” refers to variants of the SMN polypeptide of nucleic acid sequence which exist naturally within the defined taxonomic group, i.e., human. Typically, when referring to “naturally occurring variants” of a polynucleotide the term also may encompass any allelic variant of the SMN-encoding genomic DNA resulting from chromosomal translocation or duplication, and the RNA, such as mRNA derived therefrom. “Naturally occurring variants” may also include variants derived from alternative splicing of the SMN2 mRNA. When referenced to a specific polypeptide sequence, e.g., the term also includes naturally occurring forms of the protein which may therefore be processed, e.g. by co- or post-translational modifications, such as signal peptide cleavage, proteolytic cleavage, glycosylation, etc.

### Sequences

The oligomers comprise or consist of a contiguous nucleotide sequence which corresponds to the reverse complement of a nucleotide sequence present in NG_{—}008728. Thus, for example, the oligomer may comprise or consist of a sequence selected from the group consisting of SEQ ID NOS: 1-83, wherein said oligomer (or contiguous nucleotide portion thereof) may optionally have one, two, or three mismatches against said selected sequence.

The oligomer may comprise or consist of a contiguous nucleotide sequence which is fully complementary (100% complementary) to the equivalent region of a nucleic acid which encodes a human SMN (e.g., Gen Bank accession number NG_{—}008728). Thus, the oligomer can comprise or consist of an antisense nucleotide sequence. However, in some embodiments, the oligomer may tolerate 1, 2, 3, or 4 (or more) mismatches, when hybridising to the target sequence and still sufficiently bind to the target to show the desired effect, i.e. modulation of splicing of the target. Mismatches may, for example, be compensated for by increased length of the oligomer nucleotide sequence and/or an increased number of nucleotide analogues, such as LNA, present within the nucleotide sequence.

In some embodiments, the contiguous nucleotide sequence comprises no more than 3, such as no more than 2 mismatches when hybridizing to the target sequence, such as to the corresponding region of a nucleic acid which encodes a human SMN. In some embodiments, the contiguous nucleotide sequence comprises no more than a single mismatch when hybridizing to the target sequence, such as the corresponding region of a nucleic acid which encodes a human SMN.

The nucleotide sequence of the oligomer of the invention is preferably at least 80% homologous to a corresponding sequence selected from the group consisting of SEQ ID NOS: 1-83, such as at least 85%, at least 90%, at least 91%, at least 92%, at least 93%, at least 94%, at least 95%, at least 96% homologous, at least 97% homologous, at least 98% homologous, or at least 99% homologous, such as 100% homologous (identical).

The nucleotide sequence of the oligomer of the invention is preferably at least 80% homologous to the reverse complement of a corresponding sequence present in NG_{—}008728, such as at least 85%, at least 90%, at least 91%, at least 92%, at least 93%, at least 94%, at least 95%, at least 96% homologous, at least 97% homologous, at least 98% homologous, or at least 99% homologous, such as 100% homologous (identical).

The nucleotide sequence of the oligomer of the invention is preferably at least 80% complementary to a sub-sequence present in NG_{—}008728, such as at least 85%, at least 90%, at least 91%, at least 92%, at least 93%, at least 94%, at least 95%, at least 96% complementary, at least 97% complementary, at least 98% complementary, or at least 99% complementary, such as 100% complementary (perfectly complementary).

In some embodiments the oligomer (or contiguous nucleotide portion thereof) is selected from, or comprises, one of the sequences selected from the group consisting of SEQ ID NOS: 1-83, or a sub-sequence of at least 10 contiguous nucleotides thereof, wherein said oligomer may optionally comprise one, two, or three mismatches when compared to the sequence.

In some embodiments the oligomer or sub-sequence may consist of 11, 12, 13, 14, 15, 16, 17, 18, 19, 20, 21, 22, 23, 24, 25, 26, 27, 28, or 29 contiguous nucleotides, such as from 12-22, such as from 12-18 nucleotides. In some embodiments, the oligomer is 16 nucleotides in length and has the sequence of one of SEQ ID NOS: 1-20, 22, 24, 26, 28, or 30-83. In still other embodiments, the oligomer is 12 nucleotides in length and has the sequence of SEQ ID NOs: 21, 23, 25, 27, or 29.

Suitably, in some embodiments, the sub-sequence is of the same length as the contiguous nucleotide sequence of the oligomer of the invention. However, it is recognised that, in some embodiments the nucleotide sequence of the oligomer may comprise additional 5′ or 3′ nucleotides, such as, independently, 1, 2, 3, 4 or 5 additional nucleotides 5′ and/or 3′, which are non-complementary to the target sequence.

In some embodiments the oligomer according to the invention comprises or consists of a nucleotide sequence according to SEQ ID NO: 1, or a sub-sequence thereof.

In some embodiments the oligomer according to the invention comprises or consists of a nucleotide sequence according to SEQ ID NO: 2, or a sub-sequence thereof.

In some embodiments the oligomer according to the invention comprises or consists of a nucleotide sequence according to SEQ ID NO: 3, or a sub-sequence thereof.

In some embodiments the oligomer according to the invention comprises or consists of a nucleotide sequence according to SEQ ID NO: 4, or a sub-sequence thereof.

In some embodiments the oligomer according to the invention comprises or consists of a nucleotide sequence according to SEQ ID NO: 5 or a sub-sequence thereof.

In some embodiments the oligomer according to the invention comprises or consists of a nucleotide sequence according to SEQ ID NO: 6 or a sub-sequence thereof.

In some embodiments the oligomer according to the invention comprises or consists of a nucleotide sequence according to SEQ ID NO: 7 or a sub-sequence thereof.

In some embodiments the oligomer according to the invention comprises or consists of a nucleotide sequence according to SEQ ID NO: 8 or a sub-sequence thereof.

In some embodiments the oligomer according to the invention comprises or consists of a nucleotide sequence according to SEQ ID NO: 9 or a sub-sequence thereof.

In some embodiments the oligomer according to the invention comprises or consists of a nucleotide sequence according to SEQ ID NO: 10, or a sub-sequence thereof.

In some embodiments the oligomer according to the invention comprises or consists of a nucleotide sequence according to SEQ ID NO: 12, or a sub-sequence thereof.

In some embodiments the oligomer according to the invention comprises or consists of a nucleotide sequence according to SEQ ID NO: 13, or a sub-sequence thereof.

In some embodiments the oligomer according to the invention comprises or consists of a nucleotide sequence according to SEQ ID NO: 14, or a sub-sequence thereof.

In some embodiments the oligomer according to the invention comprises or consists of a nucleotide sequence according to SEQ ID NO: 15, or a sub-sequence thereof.

In some embodiments the oligomer according to the invention comprises or consists of a nucleotide sequence according to SEQ ID NO: 16, or a sub-sequence thereof.

In some embodiments the oligomer according to the invention comprises or consists of a nucleotide sequence according to SEQ ID NO: 17, or a sub-sequence thereof.

In some embodiments the oligomer according to the invention comprises or consists of a nucleotide sequence according to SEQ ID NO: 18, or a sub-sequence thereof.

In some embodiments the oligomer according to the invention comprises or consists of a nucleotide sequence according to SEQ ID NO: 19, or a sub-sequence thereof.

In some embodiments the oligomer according to the invention comprises or consists of a nucleotide sequence according to SEQ ID NO: 20, or a sub-sequence thereof.

In some embodiments the oligomer according to the invention comprises or consists of a nucleotide sequence according to SEQ ID NO: 21, or a sub-sequence thereof.

In some embodiments the oligomer according to the invention comprises or consists of a nucleotide sequence according to SEQ ID NO: 22, or a sub-sequence thereof.

In some embodiments the oligomer according to the invention comprises or consists of a nucleotide sequence according to SEQ ID NO: 23, or a sub-sequence thereof.

In some embodiments the oligomer according to the invention comprises or consists of a nucleotide sequence according to SEQ ID NO: 24, or a sub-sequence thereof.

In some embodiments the oligomer according to the invention comprises or consists of a nucleotide sequence according to SEQ ID NO: 25, or a sub-sequence thereof.

In some embodiments the oligomer according to the invention comprises or consists of a nucleotide sequence according to SEQ ID NO: 26, or a sub-sequence thereof.

In some embodiments the oligomer according to the invention comprises or consists of a nucleotide sequence according to SEQ ID NO: 27, or a sub-sequence thereof.

In some embodiments the oligomer according to the invention comprises or consists of a nucleotide sequence according to SEQ ID NO: 28, or a sub-sequence thereof.

In some embodiments the oligomer according to the invention comprises or consists of a nucleotide sequence according to SEQ ID NO: 29, or a sub-sequence thereof.

In some embodiments the oligomer according to the invention comprises or consists of a nucleotide sequence according to SEQ ID NO: 30, or a sub-sequence thereof.

In some embodiments the oligomer according to the invention comprises or consists of a nucleotide sequence according to SEQ ID NO: 31, or a sub-sequence thereof.

In some embodiments the oligomer according to the invention comprises or consists of a nucleotide sequence according to SEQ ID NO: 32, or a sub-sequence thereof.

In some embodiments the oligomer according to the invention comprises or consists of a nucleotide sequence according to SEQ ID NO: 33, or a sub-sequence thereof.

In some embodiments the oligomer according to the invention comprises or consists of a nucleotide sequence according to SEQ ID NO: 34, or a sub-sequence thereof.

In some embodiments the oligomer according to the invention comprises or consists of a nucleotide sequence according to SEQ ID NO: 35, or a sub-sequence thereof.

In some embodiments the oligomer according to the invention comprises or consists of a nucleotide sequence according to SEQ ID NO: 36, or a sub-sequence thereof.

In some embodiments the oligomer according to the invention comprises or consists of a nucleotide sequence according to SEQ ID NO: 37, or a sub-sequence thereof.

In some embodiments the oligomer according to the invention comprises or consists of a nucleotide sequence according to SEQ ID NO: 38, or a sub-sequence thereof.

In some embodiments the oligomer according to the invention comprises or consists of a nucleotide sequence according to SEQ ID NO: 39, or a sub-sequence thereof.

In some embodiments the oligomer according to the invention comprises or consists of a nucleotide sequence according to SEQ ID NO: 40, or a sub-sequence thereof.

In some embodiments the oligomer according to the invention comprises or consists of a nucleotide sequence according to SEQ ID NO: 41, or a sub-sequence thereof.

In some embodiments the oligomer according to the invention comprises or consists of a nucleotide sequence according to SEQ ID NO: 42, or a sub-sequence thereof.

In some embodiments the oligomer according to the invention comprises or consists of a nucleotide sequence according to SEQ ID NO: 43, or a sub-sequence thereof.

In some embodiments the oligomer according to the invention comprises or consists of a nucleotide sequence according to SEQ ID NO: 44, or a sub-sequence thereof.

In some embodiments the oligomer according to the invention comprises or consists of a nucleotide sequence according to SEQ ID NO: 45, or a sub-sequence thereof.

In some embodiments the oligomer according to the invention comprises or consists of a nucleotide sequence according to SEQ ID NO: 46, or a sub-sequence thereof.

In some embodiments the oligomer according to the invention comprises or consists of a nucleotide sequence according to SEQ ID NO: 47, or a sub-sequence thereof.

In some embodiments the oligomer according to the invention comprises or consists of a nucleotide sequence according to SEQ ID NO: 48, or a sub-sequence thereof.

In some embodiments the oligomer according to the invention comprises or consists of a nucleotide sequence according to SEQ ID NO: 49, or a sub-sequence thereof.

In some embodiments the oligomer according to the invention comprises or consists of a nucleotide sequence according to SEQ ID NO: 50, or a sub-sequence thereof.

In some embodiments the oligomer according to the invention comprises or consists of a nucleotide sequence according to SEQ ID NO: 51, or a sub-sequence thereof.

In some embodiments the oligomer according to the invention comprises or consists of a nucleotide sequence according to SEQ ID NO: 52, or a sub-sequence thereof.

In some embodiments the oligomer according to the invention comprises or consists of a nucleotide sequence according to SEQ ID NO: 53, or a sub-sequence thereof.

In some embodiments the oligomer according to the invention comprises or consists of a nucleotide sequence according to SEQ ID NO: 54, or a sub-sequence thereof.

In some embodiments the oligomer according to the invention comprises or consists of a nucleotide sequence according to SEQ ID NO: 55, or a sub-sequence thereof.

In some embodiments the oligomer according to the invention comprises or consists of a nucleotide sequence according to SEQ ID NO: 56, or a sub-sequence thereof.

In some embodiments the oligomer according to the invention comprises or consists of a nucleotide sequence according to SEQ ID NO: 57, or a sub-sequence thereof.

In some embodiments the oligomer according to the invention comprises or consists of a nucleotide sequence according to SEQ ID NO: 58, or a sub-sequence thereof.

In some embodiments the oligomer according to the invention comprises or consists of a nucleotide sequence according to SEQ ID NO: 59, or a sub-sequence thereof.

In some embodiments the oligomer according to the invention comprises or consists of a nucleotide sequence according to SEQ ID NO: 60, or a sub-sequence thereof.

In some embodiments the oligomer according to the invention comprises or consists of a nucleotide sequence according to SEQ ID NO: 61, or a sub-sequence thereof.

In some embodiments the oligomer according to the invention comprises or consists of a nucleotide sequence according to SEQ ID NO: 62, or a sub-sequence thereof.

In some embodiments the oligomer according to the invention comprises or consists of a nucleotide sequence according to SEQ ID NO: 63, or a sub-sequence thereof.

In some embodiments the oligomer according to the invention comprises or consists of a nucleotide sequence according to SEQ ID NO: 64, or a sub-sequence thereof.

In some embodiments the oligomer according to the invention comprises or consists of a nucleotide sequence according to SEQ ID NO: 65, or a sub-sequence thereof.

In some embodiments the oligomer according to the invention comprises or consists of a nucleotide sequence according to SEQ ID NO: 66, or a sub-sequence thereof.

In some embodiments the oligomer according to the invention comprises or consists of a nucleotide sequence according to SEQ ID NO: 67, or a sub-sequence thereof.

In some embodiments the oligomer according to the invention comprises or consists of a nucleotide sequence according to SEQ ID NO: 68, or a sub-sequence thereof.

In some embodiments the oligomer according to the invention comprises or consists of a nucleotide sequence according to SEQ ID NO: 69, or a sub-sequence thereof.

In some embodiments the oligomer according to the invention comprises or consists of a nucleotide sequence according to SEQ ID NO: 70, or a sub-sequence thereof.

In some embodiments the oligomer according to the invention comprises or consists of a nucleotide sequence according to SEQ ID NO: 71, or a sub-sequence thereof.

In some embodiments the oligomer according to the invention comprises or consists of a nucleotide sequence according to SEQ ID NO: 72, or a sub-sequence thereof.

In some embodiments the oligomer according to the invention comprises or consists of a nucleotide sequence according to SEQ ID NO: 73, or a sub-sequence thereof.

In some embodiments the oligomer according to the invention comprises or consists of a nucleotide sequence according to SEQ ID NO: 74, or a sub-sequence thereof.

In some embodiments the oligomer according to the invention comprises or consists of a nucleotide sequence according to SEQ ID NO: 75, or a sub-sequence thereof.

In some embodiments the oligomer according to the invention comprises or consists of a nucleotide sequence according to SEQ ID NO: 76, or a sub-sequence thereof.

In some embodiments the oligomer according to the invention comprises or consists of a nucleotide sequence according to SEQ ID NO: 77, or a sub-sequence thereof.

In some embodiments the oligomer according to the invention comprises or consists of a nucleotide sequence according to SEQ ID NO: 78, or a sub-sequence thereof.

In some embodiments the oligomer according to the invention comprises or consists of a nucleotide sequence according to SEQ ID NO: 79, or a sub-sequence thereof.

In some embodiments the oligomer according to the invention comprises or consists of a nucleotide sequence according to SEQ ID NO: 80, or a sub-sequence thereof.

In some embodiments the oligomer according to the invention comprises or consists of a nucleotide sequence according to SEQ ID NO: 81, or a sub-sequence thereof.

In some embodiments the oligomer according to the invention comprises or consists of a nucleotide sequence according to SEQ ID NO: 82, or a sub-sequence thereof.

In some embodiments the oligomer according to the invention comprises or consists of a nucleotide sequence according to SEQ ID NO: 83, or a sub-sequence thereof.

In determining the degree of “complementarity” between oligomers of the invention (or regions thereof) and the target region of the nucleic acid which encodes human SMN, such as those disclosed herein, the degree of “complementarity” is expressed as the percentage identity (percentage homology) between the sequence of the oligomer (or region thereof) and the sequence of the reverse complement of the target region that best aligns therewith. The percentage is calculated by counting the number of aligned bases that are identical between the 2 sequences, dividing by the total number of contiguous monomers in the oligomer, and multiplying by 100. In such a comparison, if gaps exist, it is preferable that such gaps are merely mismatches rather than areas where the number of monomers within the gap differs between the oligomer of the invention and the target region.

Similarly, the degree of “homology” or “identity” is expressed as the percentage identity (percentage homology) between the sequence of the oligomer (or region thereof) and the sequence of the target region that best aligns therewith. As used herein, the terms “homologous” and “homology” are interchangeable with the terms “identical” and “identity”.

The terms “corresponding to” and “corresponds to” refer to the comparison between the nucleotide sequence of the oligomer (i.e. the nucleobase or base sequence) or contiguous nucleotide sequence and the equivalent contiguous nucleotide sequence of a further sequence selected from either i) a sub-sequence of the reverse complement of the nucleic acid target, such as the nucleic acid which encodes the SMN protein, such as Genbank Acc. No. NG_{—}008728 and/or ii) the nucleotide sequences provided herein such as the group consisting of SEQ ID NOS: 1-83, or sub-sequence thereof. Nucleotide analogues are compared directly to their equivalent or corresponding nucleotides. A first sequence which corresponds to a further sequence under i) or ii) typically is identical to that sequence over the length of the first sequence (such as the contiguous nucleotide sequence) or, as described herein may, in some embodiments, is at least 80% homologous to a corresponding sequence, such as at least 85%, at least 90%, at least 91%, at least 92%, at least 93%, at least 94%, at least 95%, at least 96%, at least 97%, at least 98%, or at least 99% homologous, such as 100% homologous (identical).

The terms “corresponding nucleotide analogue” and “corresponding nucleotide” are intended to indicate that the nucleobase in the nucleotide analogue and the naturally occurring nucleotide are identical. For example, when the 2-deoxyribose unit of the nucleotide is linked to an adenine, the “corresponding nucleotide analogue” contains a pentose unit (different from 2-deoxyribose) linked to an adenine.

The terms “reverse complement”, “reverse complementary” and “reverse complementarity” as used herein are interchangeable with the terms “complement”, “complementary” and “complementarity”.

### Length

The oligomers may comprise or consist of a contiguous nucleotide sequence of a total of 10, 11, 12, 13, 14, 15, 16, 17, 18, 19, 20, 21, 22, 23, 24, 25, 26, 27, 28, 29 or 30 contiguous nucleotides in length.

In some embodiments, the oligomers comprise or consist of a contiguous nucleotide sequence of a total of from 10 to 22 nucleotides, such as 12-18, 13-17 or 12-16 nucleotides, such as 13, 14, 15, or 16 contiguous nucleotides in length.

In some embodiments, the oligomers comprise or consist of a contiguous nucleotide sequence of a total of 10, 11, 12, 13, or 14 contiguous nucleotides in length.

In some embodiments, the oligomer according to the invention consists of no more than 22 nucleotides, such as no more than 20 nucleotides, such as no more than 18 nucleotides, such as 15, 16 or 17 nucleotides. In some embodiments the oligomer of the invention comprises less than 20 nucleotides. It should be understood that when a range is given for an oligomer, or contiguous nucleotide sequence length it includes the lower and upper lengths provided in the range, for example from (or between) 10-30, includes both 10 and 30.

### Nucleosides and Nucleoside Analogues

In some embodiments, the terms “nucleoside analogue” and “nucleotide analogue” are used interchangeably.

The term “nucleotide” as used herein, refers to a glycoside comprising a sugar moiety, a base moiety and a covalently linked group (linkage group), such as a phosphate or phosphorothioate internucleotide linkage group, and covers both naturally occurring nucleotides, such as DNA or RNA, and non-naturally occurring nucleotides comprising modified sugar and/or base moieties, which are also referred to as “nucleotide analogues” herein. Herein, a single nucleotide (unit) may also be referred to as a monomer or nucleic acid unit.

In field of biochemistry, the term “nucleoside” is commonly used to refer to a glycoside comprising a sugar moiety and a base moiety, and may therefore be used when referring to the nucleotide units, which are covalently linked by the internucleotide linkages between the nucleotides of the oligomer. In the field of biotechnology, the term “nucleotide” is often used to refer to a nucleic acid monomer or unit, and as such in the context of an oligonucleotide may refer to the base—such as the “nucleotide sequence”, typically refer to the nucleobase sequence (i.e. the presence of the sugar backbone and internucleoside linkages are implicit). Likewise, particularly in the case of oligonucleotides where one or more of the internucleoside linkage groups are modified, the term “nucleotide” may refer to a “nucleoside” for example the term “nucleotide” may be used, even when specifying the presence or nature of the linkages between the nucleosides.

As one of ordinary skill in the art would recognise, the 5′ terminal nucleotide of an oligonucleotide does not comprise a 5′ internucleotide linkage group, although may or may not comprise a 5′ terminal group.

Non-naturally occurring nucleotides include nucleotides which have modified sugar moieties, such as bicyclic nucleotides or 2′ modified nucleotides, such as 2′ substituted nucleotides.

“Nucleotide analogues” are variants of natural nucleotides, such as DNA or RNA nucleotides, by virtue of modifications in the sugar and/or base moieties. Analogues could in principle be merely “silent” or “equivalent” to the natural nucleotides in the context of the oligonucleotide, i.e. have no functional effect on the way the oligonucleotide works to inhibit target gene expression. Such “equivalent” analogues may nevertheless be useful if, for example, they are easier or cheaper to manufacture, or are more stable to storage or manufacturing conditions, or represent a tag or label. Preferably, however, the analogues will have a functional effect on the way in which the oligomer works to inhibit expression; for example by producing increased binding affinity to the target and/or increased resistance to intracellular nucleases and/or increased ease of transport into the cell. Specific examples of nucleoside analogues are described by e.g. Freier & Altmann; Nucl. Acid Res., 1997, 25, 4429-4443 and Uhlmann; Curr. Opinion in Drug Development, 2000, 3(2), 293-213, and in Scheme 1:

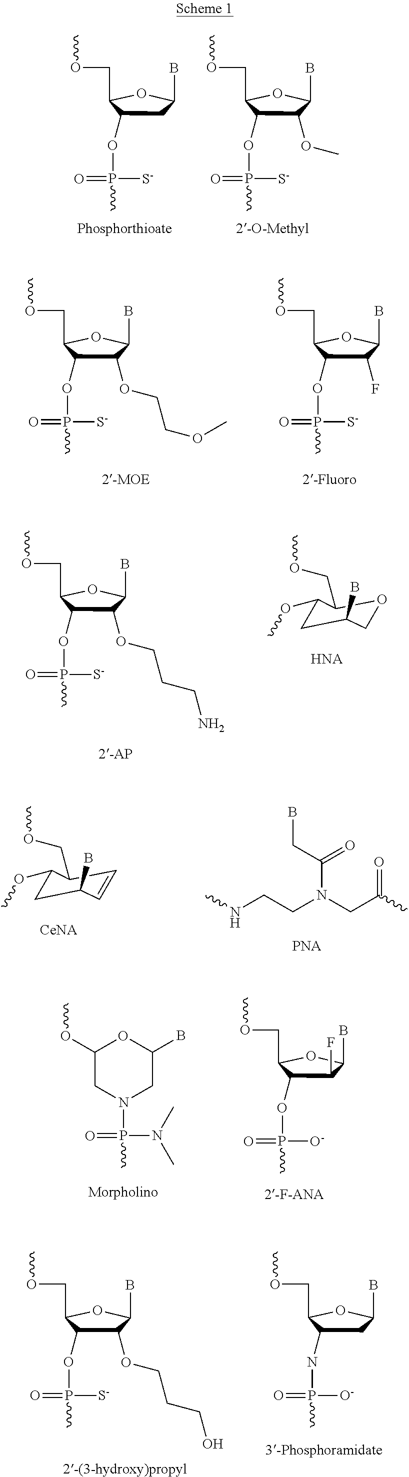

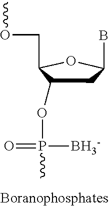

The oligomer may thus comprise or consist of a simple sequence of naturally occurring nucleotides—preferably 2′-deoxynucleotides (referred to here generally as “DNA”), but also possibly ribonucleotides (referred to here generally as “RNA”), or a combination of such naturally occurring nucleotides and one or more non-naturally occurring nucleotides, i.e. nucleotide analogues. Such nucleotide analogues may suitably enhance the affinity of the oligomer for the target sequence.

Examples of suitable and preferred nucleotide analogues are provided by WO2007/031091 or are referenced therein.

Incorporation of affinity-enhancing nucleotide analogues in the oligomer, such as LNA or 2′-substituted sugars, can allow the size of the specifically binding oligomer to be reduced, and may also reduce the upper limit to the size of the oligomer before non-specific or aberrant binding takes place.

In some embodiments, the oligomer comprises at least 1 nucleoside analogue. In some embodiments the oligomer comprises at least 2 nucleotide analogues. In some embodiments, the oligomer comprises from 3-8 nucleotide analogues, e.g. 6 or 7 nucleotide analogues. In the by far most preferred embodiments, at least one of said nucleotide analogues is a locked nucleic acid (LNA); for example at least 3 or at least 4, or at least 5, or at least 6, or at least 7, or 8, of the nucleotide analogues may be LNA. In some embodiments all the nucleotide analogues may be LNA; in other embodiments approximately half of the nucleotide analogues may be LNA.

It will be recognised that when referring to a preferred nucleotide sequence motif or nucleotide sequence, which consists of only nucleotides, the oligomers of the invention which are defined by that sequence may comprise a corresponding nucleotide analogue (that is, having the same nucleobase) in place of one or more of the nucleotides present in said sequence, such as LNA units or other nucleotide analogues, which raise the duplex stability/Tₘ of the oligomer/target duplex (i.e. affinity enhancing nucleotide analogues).

In some embodiments, any mismatches between the nucleotide sequence of the oligomer and the target sequence are preferably found in regions outside the affinity enhancing nucleotide analogues, and/or at the site of non modified such as DNA nucleotides in the oligonucleotide, and/or in regions which are 5′ or 3′ to the contiguous nucleotide sequence.

Examples of such modification of the nucleotide include modifying the sugar moiety to provide a 2′-substituent group or to produce a bridged (locked nucleic acid) structure which enhances binding affinity and may also provide increased nuclease resistance.

A preferred nucleotide analogue is LNA, such as oxy-LNA (such as beta-D-oxy-LNA, and alpha-L-oxy-LNA), and/or amino-LNA (such as beta-D-amino-LNA and alpha-L-amino-LNA) and/or thio-LNA (such as beta-D-thio-LNA and alpha-L-thio-LNA) and/or ENA (such as beta-D-ENA and alpha-L-ENA). Most preferred is beta-D-oxy-LNA.

In some embodiments the nucleotide analogues present within the oligomer of the invention are independently selected from, for example: 2′-O-alkyl-RNA units, 2′-amino-DNA units, 2′-fluoro-DNA units, LNA units, arabino nucleic acid (ANA) units, 2′-fluoro-ANA units, HNA units, INA (intercalating nucleic acid—Christensen, 2002. Nucl. Acids. Res. 2002 30: 4918-4925, hereby incorporated by reference) units and 2′MOE units. In some embodiments there is only one of the above types of nucleotide analogues present in the oligomer of the invention, or contiguous nucleotide sequence thereof.

In some embodiments the nucleotide analogues are 2′-O-methoxyethyl-RNA (2′MOE), 2′-fluoro-DNA monomers or LNA nucleotide analogues, and as such the oligonucleotide of the invention may comprise nucleotide analogues which are independently selected from these three types of analogue, or may comprise only one type of analogue selected from the three types. In some embodiments at least one of said nucleotide analogues is 2′-MOE-RNA, such as 2, 3, 4, 5, 6, 7, 8, 9 or 10 2′-MOE-RNA nucleotide units. In some embodiments at least one of said nucleotide analogues is 2′-fluoro DNA, such as 2, 3, 4, 5, 6, 7, 8, 9 or 10 2′-fluoro-DNA nucleotide units.

In some embodiments, the oligomer according to the invention comprises at least one Locked Nucleic Acid (LNA) unit, such as 1, 2, 3, 4, 5, 6, 7, or 8 LNA units, such as from 3-7 or 4 to 8 LNA units, or 3, 4, 5, 6 or 7 LNA units. In some embodiments, all the nucleotide analogues are LNA. In some embodiments, the oligomer may comprise both beta-D-oxy-LNA, and one or more of the following LNA units: thio-LNA, amino-LNA, oxy-LNA, and/or ENA in either the beta-D or alpha-L configurations or combinations thereof. In some embodiments all LNA cytosine units are 5′ methyl-cytosine. In some embodiments of the invention, the oligomer may comprise both LNA and DNA units. Preferably the combined total of LNA and DNA units is 10-25, such as 10-24, preferably 10-20, such as 10-18, even more preferably 12-16. In some embodiments of the invention, the nucleotide sequence of the oligomer, such as the contiguous nucleotide sequence consists of at least one LNA and the remaining nucleotide units are DNA units. In some embodiments the oligomer comprises only LNA nucleotide analogues and naturally occurring nucleotides (such as RNA or DNA, most preferably DNA nucleotides), optionally with modified internucleotide linkages such as phosphorothioate.

The term “nucleobase” refers to the base moiety of a nucleotide and covers both naturally occurring as well as non-naturally occurring variants. Thus, “nucleobase” covers not only the known purine and pyrimidine heterocycles but also heterocyclic analogues and tautomers thereof.

Examples of nucleobases include, but are not limited to adenine, guanine, cytosine, thymidine, uracil, xanthine, hypoxanthine, 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

In some embodiments, at least one of the nucleobases present in the oligomer is a modified nucleobase selected from the group consisting of 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

### LNA

The term “LNA” refers to a bicyclic nucleoside analogue, known as “Locked Nucleic Acid”. It may refer to an LNA monomer, or, when used in the context of an “LNA oligonucleotide”, LNA refers to an oligonucleotide containing one or more such bicyclic nucleotide analogues. LNA nucleotides are characterised by the presence of a linker group (such as a bridge) between C2′ and C4′ of the ribose sugar ring—for example as shown as the biradical R^{4*}-R^{2*} as described below.

The LNA used in the oligonucleotide compounds of the invention preferably has the structure of the general formula I

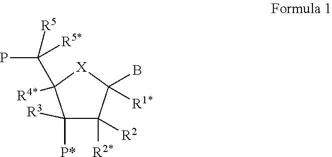

wherein for all chiral centers, asymmetric groups may be found in either R or S orientation;

wherein X is selected from —O—, —S—, —N(R^{N*})—, —C(R⁶R^{6*})—, such as, in some embodiments —O—;

B is selected from hydrogen, optionally substituted C₁₋₄-alkoxy, optionally substituted C₁₋₄-alkyl, optionally substituted C₁₋₄-acyloxy, nucleobases including naturally occurring and nucleobase analogues, DNA intercalators, photochemically active groups, thermochemically active groups, chelating groups, reporter groups, and ligands; preferably, B is a nucleobase or nucleobase analogue;

P designates an internucleotide linkage to an adjacent monomer, or a 5′-terminal group, such internucleotide linkage or 5′-terminal group optionally including the substituent R⁵ or equally applicable the substituent R^{5*};

P* designates an internucleotide linkage to an adjacent monomer, or a 3′-terminal group;

R^{4*} and R^{2*} together designate a bivalent linker group consisting of 1-4 groups/atoms selected from —C(R^{a}R^{b})—, —C(R^{a})═C(R^{b})—, —C(R^{a})═N—, —O—, —Si(R^{a})₂—, —S—, —SO₂—, —N(R^{a})—, and >C═Z, wherein Z is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, optionally substituted C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, hetero-aryloxy-carbonyl, heteroaryloxy, heteroarylcarbonyl, amino, mono- and di(C₁₋₆-alkyl)amino, carbamoyl, mono- and di(C₁₋₆-alkyl)amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂), wherein for all chiral centers, asymmetric groups may be found in either R or S orientation, and;

each of the substituents R^{1*}, R², R³, R⁵, R^{5*}, R⁶ and R^{6*}, which are present is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene; wherein R^{N} is selected from hydrogen and C₁₋₄-alkyl, and where two adjacent (non-geminal) substituents may designate an additional bond resulting in a double bond; and R^{N*}, when present and not involved in a biradical, is selected from hydrogen and C₁₋₄-alkyl; and basic salts and acid addition salts thereof. For all chiral centers, asymmetric groups may be found in either R or S orientation.

In some embodiments, R^{4*} and R^{2*} together designate a biradical consisting of a groups selected from the group consisting of C(R^{a}R^{b})—C(R^{a}R^{b})—, C(R^{a}R^{b})—O—, C(R^{a}R^{b})—NR^{a}—, C(R^{a}R^{b})—S—, and C(R^{a}R^{b})—C(R^{a}R^{b})—O—, wherein each R^{a} and R^{b} may optionally be independently selected. In some embodiments, R^{a} and R^{b} may be, optionally independently selected from the group consisting of hydrogen and C₁₋₆alkyl, such as methyl, such as hydrogen.

In some embodiments, R^{4*} and R^{2*} together designate the biradical —O—CH(CH₂OCH₃)-(2′O-methoxyethyl bicyclic nucleic acid—Seth at al., 2010, J. Org. Chem.)—in either the R- or S-configuration.

In some embodiments, R^{4*} and R^{2*} together designate the biradical —O—CH(CH₂CH₃)-(2′O-ethyl bicyclic nucleic acid—Seth at al., 2010, J. Org. Chem.).—in either the R- or S-configuration.

In some embodiments, R^{4*} and R^{2*} together designate the biradical —O—CH(CH₃)—.—in either the R- or S-configuration. In some embodiments, R^{4*} and R^{2*} together designate the biradical —O—CH₂—O—CH₂— (Seth at al., 2010, J. Org. Chem.).

In some embodiments, R^{4*} and R^{2*} together designate the biradical —O—NR—CH₃— (Seth at al., 2010, J. Org. Chem.).

In some embodiments, the LNA units have a structure selected from the following group:

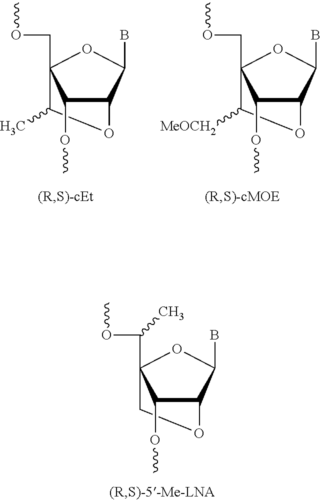

In some embodiments, R^{1*}, R², R³, R⁵, R^{5*} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. For all chiral centers, asymmetric groups may be found in either R or S orientation.

In some embodiments, R^{1*}, R², R³, R⁵, R^{5*} are hydrogen.

In some embodiments, R^{1*}, R², R³ are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. For all chiral centers, asymmetric groups may be found in either R or S orientation.

In some embodiments, R^{1*}, R², R³ are hydrogen.

In some embodiments, R⁵ and R^{5*} are each independently selected from the group consisting of H, —CH₃, —CH₂—CH₃, —CH₂—O—CH₃, and —CH═CH₂. Suitably in some embodiments, either R⁵ or R^{5*} are hydrogen, where as the other group (R⁵ or R^{5*} respectively) is selected from the group consisting of C₁₋₅ alkyl, C₂₋₆ alkenyl, C₂₋₆ alkynyl, substituted C₁₋₆ alkyl, substituted C₂₋₆ alkenyl, substituted C₂₋₆ alkynyl or substituted acyl (—C(═O)—); wherein each substituted group is mono or poly substituted with substituent groups independently selected from halogen, C₁₋₆ alkyl, substituted C₁₋₆ alkyl, C₂₋₆ alkenyl, substituted C₂₋₆ alkenyl, C₂₋₆ alkynyl, substituted C₂₋₆ alkynyl, OJ₁, SJ₁, NJ₁J₂, N₃, COOJ₁, CN, O—C(═O)NJ₁J₂, N(H)C(═NH)NJ, J₂ or N(H)C(═X)N(H)J₂ wherein X is O or S; and each J₁ and J₂ is, independently, H, C₁₋₆ alkyl, substituted C₁₋₆ alkyl, C₂₋₆ alkenyl, substituted C₂₋₆ alkenyl, C₂₋₆ alkynyl, substituted C₂₋₆ alkynyl, C₁₋₆ aminoalkyl, substituted C₁₋₆ aminoalkyl or a protecting group. In some embodiments either R⁵ or R^{5*} is substituted C₁₋₆ alkyl. In some embodiments either R⁵ or R^{5*} is substituted methylene wherein preferred substituent groups include one or more groups independently selected from F, NJ₁J₂, N₃, CN, OJ₁, SJ₁, O—C(═O)NJ₁J₂, N(H)C(═NH)NJ, J₂ or N(H)C(O)N(H)J₂. In some embodiments each J₁ and J₂ is, independently H or C₁₋₆ alkyl. In some embodiments either R⁵ or R^{5*} is methyl, ethyl or methoxymethyl. In some embodiments either R⁵ or R^{5*} is methyl. In a further embodiment either R⁵ or R^{5*} is ethylenyl. In some embodiments either R⁵ or R^{5*} is substituted acyl. In some embodiments either R⁵ or R^{5*} is C(═O)NJ₁J₂. For all chiral centers, asymmetric groups may be found in either R or S orientation. Such 5′ modified bicyclic nucleotides are disclosed in WO 2007/134181, which is hereby incorporated by reference in its entirety.

In some embodiments B is a nucleobase, including nucleobase analogues and naturally occurring nucleobases, such as a purine or pyrimidine, or a substituted purine or substituted pyrimidine, such as a nucleobase referred to herein, such as a nucleobase selected from the group consisting of adenine, cytosine, thymine, adenine, uracil, and/or a modified or substituted nucleobase, such as 5-thiazolo-uracil, 2-thio-uracil, 5-propynyluracil, 2′ thio-thymine, 5-methyl cytosine, 5-thiozolo-cytosine, 5-propynyl-cytosine, and 2,6-diaminopurine.

In some embodiments, R^{4*} and R^{2*} together designate a biradical selected from —C(R^{a}R^{b})—O—, —C(R^{a}R^{b})—C(R^{c}R^{d})—O—, —C(R^{a}R^{b})—(R^{c}R^{d})—C(R^{e}R^{f})—O—, —C(R^{a}R^{b})—O—C(R^{c}R^{d})—, —C(R^{a}R^{b})—O—C(R^{c}R^{d})—O—, —C(R^{a}R^{b})—C(R^{c}R^{d})—, —C(R^{a}R^{b})—C(R^{c}R^{d})—C(R^{e}R^{f})—, —C(R^{a})═C(R^{b})—C(R^{c}R^{d})—, —C(R^{a}R^{b})—N(R^{c})—, —C(R^{a}R^{b})—C(R^{c}R^{d})—N(R^{e})—, —C(R^{a}R^{b})—N(R^{c})—O—, and —C(R^{a}R^{b})—S—, —C(R^{a}-R^{b})—C(R^{c}R^{d})—S—, wherein R^{a}, R^{b}, R^{c}, R^{d}, R^{e}, and R^{f} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂). For all chiral centers, asymmetric groups may be found in either R or S orientation.

In a further embodiment R^{4*} and R^{2*} together designate a biradical (bivalent group) selected from —CH₂—O—, —CH₂—S—, —CH₂—NH—, —CH₂—N(CH₃)—, —CH₂—CH₂—O—, —CH₂—CH(CH₃)—, —CH₂—CH₂—S—, —CH₂—CH₂—NH—, —CH₂—CH₂—CH₂—, —CH₂—CH₂—CH₂—O—, —CH₂—CH₂—CH(CH₃)—, —CH═CH—CH₂—, —CH₂—O—CH₂—O—, —CH₂—NH—O—, —CH₂—N(CH₃)—O—, —CH₂—O—CH₂—, —CH(CH₃)—O—, and —CH(CH₂—O—CH₃)—O—, and/or, —CH₂—CH₂—, and —CH═CH—For all chiral centers, asymmetric groups may be found in either R or S orientation.

In some embodiments, R^{4*} and R^{2*} together designate the biradical C(R^{a}R^{b})—N(R^{c})—O—, wherein R^{a} and R^{b} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen, and; wherein R^{c} is selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen.

In some embodiments, R^{4*} and R^{2*} together designate the biradical C(R^{a}R^{b})—O—C(R^{c}R^{d})—O—, wherein R^{a}, R^{b}, R^{c}, and R^{d} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen.

In some embodiments, R^{4*} and R^{2*} form the biradical —CH(Z)—O—, wherein Z is selected from the group consisting of C₁₋₆ alkyl, C₂₋₆ alkenyl, C₂₋₆ alkynyl, substituted C₁₋₆ alkyl, substituted C₂₋₆ alkenyl, substituted C₂₋₆ alkynyl, acyl, substituted acyl, substituted amide, thiol or substituted thio; and wherein each of the substituted groups, is, independently, mono or poly substituted with optionally protected substituent groups independently selected from halogen, oxo, hydroxyl, OJ₁, NJ₁J₂, SJ₁, N₃, OC(═X)J₁, OC(═X)NJ₁J₂, NJ³C(═X)NJ₁J₂ and CN, wherein each J₁, J₂ and J₃ is, independently, H or C₁₋₆ alkyl, and X is O, S or NJ₁. In some embodiments Z is C₁₋₆ alkyl or substituted C₁₋₆ alkyl. In some embodiments Z is methyl. In some embodiments Z is substituted C₁₋₆ alkyl. In some embodiments said substituent group is C₁₋₆ alkoxy. In some embodiments Z is CH₃OCH₂—. For all chiral centers, asymmetric groups may be found in either R or S orientation. Such bicyclic nucleotides are disclosed in U.S. Pat. No. 7,399,845 which is hereby incorporated by reference in its entirety. In some embodiments, R^{1*}, R², R³, R⁵, R^{5*} are hydrogen. In some embodiments, R^{1*}, R², R^{3*} are hydrogen, and one or both of R⁵, R^{5*} may be other than hydrogen as referred to above and in WO 2007/134181.

In some embodiments, R^{4*} and R^{2*} together designate a biradical which comprise a substituted amino group in the bridge such as consist or comprise of the biradical —CH₂—N(R^{c})—, wherein R^{c} is C₁₋₁₂ alkyloxy. In some embodiments R^{4*} and R^{2*} together designate a biradical -Cq₃q₄-NOR—, wherein q₃ and q₄ are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl; wherein each substituted group is, independently, mono or poly substituted with substituent groups independently selected from halogen, OJ₁, SJ₁, NJ₁J₂, COOJ₁, CN, O—C(═O)NJ₁J₂, N(H)C(═NH)N J₁J₂ or N(H)C(═X═N(H)J₂ wherein X is O or S; and each of J₁ and J₂ is, independently, H, C₁₋₆ alkyl, C₂₋₆ alkenyl, C₂₋₆ alkynyl, C₁₋₆ aminoalkyl or a protecting group. For all chiral centers, asymmetric groups may be found in either R or S orientation. Such bicyclic nucleotides are disclosed in WO2008/150729 which is hereby incorporated by reference in its entirety. In some embodiments, R^{1*}, R², R³, R⁵, R^{5*} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. In some embodiments, R^{1*}, R², R³, R⁵, R^{5*} are hydrogen. In some embodiments, R^{1*}, R², R³ are hydrogen and one or both of R⁵, R^{5*} may be other than hydrogen as referred to above and in WO 2007/134181. In some embodiments R^{4*} and R^{2*} together designate a biradical (bivalent group) C(R^{a}R^{b})—O—, wherein R^{a} and R^{b} are each independently halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJ₁ SJ₁, SOJ₁, SO₂J₁, NJ₁J₂, N₃, CN, C(═O)OJ₁, C(═O)NJ₁J₂, C(═O)J₁, O—C(═O)NJ₁J₂, N(H)C(═NH)NJ₁J₂, N(H)C(═O)NJ₁J₂ or N(H)C(═S)NJ₁J₂; or R^{a} and R^{b} together are ═C(q3)(q4); q₃ and q₄ are each, independently, H, halogen, C₁-C₁₂alkyl or substituted C₁-C₁₂ alkyl; each substituted group is, independently, mono or poly substituted with substituent groups independently selected from halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, OJ₁, SJ₁, NJ₁J₂, N₃, CN, C(═O)OJ₁, C(═O)NJ₁J₂, C(═O)J₁, O—C(═O)NJ₁J₂, N(H)C(═O)NJ₁J₂ or N(H)C(═S)NJ₁J₂ and; each J₁ and J₂ is, independently, H, C1-C₆ alkyl, substituted C1-C₆ alkyl, C₂-C₆ alkenyl, substituted C₂-C₆ alkenyl, C₂-C₆ alkynyl, substituted C₂-C₆ alkynyl, C1-C₆ aminoalkyl, substituted C1-C₆ aminoalkyl or a protecting group. Such compounds are disclosed in WO2009006478A, hereby incorporated in its entirety by reference.

In some embodiments, R^{4*} and R^{2*} form the biradical -Q-, wherein Q is C(q₁)(q₂)C(q₃)(q₄), C(q₁)═C(q₃), C[═C(q₁)(q₂)]—C(q₃)(q₄) or C(q₁)(q₂)—C[═C(q₃)(q₄)]; q₁, q₂, q₃, q₄ are each independently. H, halogen, C₁₋₁₂ alkyl, substituted C₁₋₁₂ alkyl, C₂₋₁₂ alkenyl, substituted C₁₋₁₂ alkoxy, OJ₁, SJ₁, SOJ₁, SO₂J₁, NJ₁J₂, N₃, CN, C(═O)OJ₁, C(═O)—NJ₁J₂, C(═O) J₁, —C(═O)NJ₁J₂, N(H)C(═NH)NJ₁J₂, N(H)C(═O)NJ₁J₂ or N(H)C(═S)NJ₁J₂; each J₁ and J₂ is, independently, H, C₁₋₆ alkyl, C₂₋₆ alkenyl, C₂₋₆ alkynyl, C₁₋₆ aminoalkyl or a protecting group; and, optionally wherein when Q is C(q₁)(q₂)(q₃)(q₄) and one of q₃ or q₄ is CH₃ then at least one of the other of q₃ or q₄ or one of q₁ and q₂ is other than H. In some embodiments, R^{1*}, R², R³, R⁵, R^{5*} are hydrogen. For all chiral centers, asymmetric groups may be found in either R or S orientation. Such bicyclic nucleotides are disclosed in WO2008/154401 which is hereby incorporated by reference in its entirety. In some embodiments, R^{1*}, R², R³, R⁵, R^{5*} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. In some embodiments, R^{1*}, R², R³, R⁵, R^{5*} are hydrogen. In some embodiments, R^{1*}, R², R³ are hydrogen and one or both of R⁵, R^{5*} may be other than hydrogen as referred to above and in WO 2007/134181 or WO2009/067647 (alpha-L-bicyclic nucleic acids analogs).

In some embodiments the LNA used in the oligonucleotide compounds of the invention preferably has the structure of the general formula II:

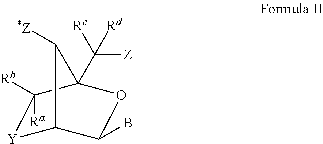

wherein Y is selected from the group consisting of —O—, —CH₂O—, —S—, —NH—, N(R^{e}) and/or —CH₂—; Z and Z* are independently selected among an internucleotide linkage, R^{H}, a terminal group or a protecting group; B constitutes a natural or non-natural nucleotide base moiety (nucleobase), and R^{H} is selected from hydrogen and C₁₋₄-alkyl; R^{a}, R^{b}R^{c}, R^{d} and R^{e} are, optionally independently, selected from the group consisting of hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂); and R^{H} is selected from hydrogen and C₁₋₄-alkyl. In some embodiments R^{a}, R^{b} R^{c}, R^{d} and R^{e} are, optionally independently, selected from the group consisting of hydrogen and C₁₋₆ alkyl, such as methyl. For all chiral centers, asymmetric groups may be found in either R or S orientation, for example, two exemplary stereochemical isomers include the beta-D and alpha-L isoforms, which may be illustrated as follows:

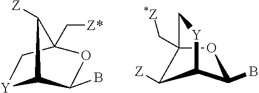

Specific exemplary LNA units are shown below:

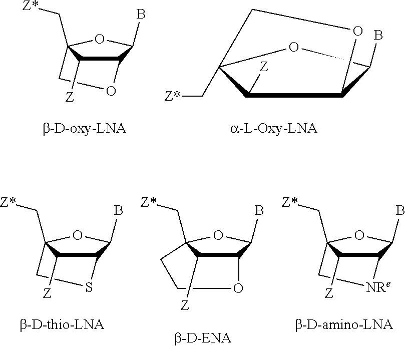

The term “thio-LNA” comprises a locked nucleotide in which Y in the general formula above is selected from S or —CH₂—S—. Thio-LNA can be in both beta-D and alpha-L-configuration.

The term “amino-LNA” comprises a locked nucleotide in which Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which Y in the general formula above represents —O—. Oxy-LNA can be in both beta-D and alpha-L-configuration.

The term “ENA” comprises a locked nucleotide in which Y in the general formula above is —CH₂—O— (where the oxygen atom of —CH₂—O— is attached to the 2′-position relative to the base B). R^{e} is hydrogen or methyl.

In some exemplary embodiments LNA is selected from beta-D-oxy-LNA, alpha-L-oxy-LNA, beta-D-amino-LNA and beta-D-thio-LNA, in particular beta-D-oxy-LNA.

### RNAse Recruitment

It is recognised that an oligomeric compound may function by recruiting an endoribonuclease (RNase), such as RNase H, or via non RNase mediated degradation of target mRNA, such as by steric hindrance of translation or by modulation of splicing. EP 1 222 309 (in particular Examples 91-95) provides in vitro methods for determining RNaseH activity, which may be used to determine the ability to recruit RNaseH.

### Modulation of Splicing

Many eukaryotic mRNA transcripts contain one or more regions, known as “introns,” which are excised from (spliced out of) a transcript before it is translated. The RNA transcript prior to splicing is referred to as pre-mRNA. The remaining (and therefore translated) regions are known as “exons” and are spliced together to form a continuous (mature) mRNA sequence. mRNA splice sites, i.e., intron-exon junctions, may also be preferred target regions, and are particularly useful in situations where aberrant splicing is implicated in disease, or where an overproduction of a particular mRNA splice product is implicated in disease. For modulation of splicing as in the instant invention, it is preferred that the oligonucleotides do not elicit RNAse H cleavage of the nucleic acid target, which would decrease the amount of target mRNA present in the cell. Instead, oligomers are designed to interfere with splicing through non-RNAse H methods, with the goal being to modulate aberrant splicing in favor of a desired splice product (in this case, full length SMN2 mRNA). Thus the level of a desired splice product (mRNA or its protein product) may actually be increased through use of antisense methods.

### Mixmer Design

Some “chimeric” oligomers, called “mixmers”, consist of an alternating composition of (i) DNA monomers or nucleoside analogue monomers recognizable and cleavable by RNase, and (ii) non-RNase recruiting nucleoside analogue monomers.

The oligonucleotides of the instant invention preferably do not elicit RNAse H, and in a preferred embodiment the oligonucleotides are “mixmers,” i.e., having a mixture of modified nucleosides which are not easily cleaved by RNAse H, and unmodified DNA units which can be cleaved by RNAse H, but unlike gapmers, have no DNA “gap” region long enough to bind and mediate RNAse H cleavage. It is currently believed that 4 to 5 contiguous DNA units are necessary for RNAse H cleavage and it is therefore preferred to have fewer than 4, more preferably fewer than 3, or fewer than 2, contiguous DNA units in an oligomer that is intended not to elicit RNAse H. As shown in Table 1, the preferred mixmers of the instant invention have LNA in every other position and two or three LNAs at the 3′ end, which are believed to stabilize the oligonucleotide and minimize RNAse H cleavage. The backbone linkages are phosphorothioate linkages.

In some embodiments, the oligomer comprises of only LNA and DNA nucleotides.

In some embodiments, the oligomer has fewer than 4 contiguous DNA units, such as fewer than 3 contiguous DNA units, such as fewer than 2 contiguous DNA units. In some embodiments the oligomer has no more than 1 or 2 contiguous DNA units.

In some embodiments, the 5′ unit of the oligomer is an LNA nucleotide. In some embodiments, the 3′ unit of the oligomer, such as the 2 3′ units is/are an LNA nucleotide.

In some embodiments, the oligomer comprises of LNA and DNA nucleotides, wherein there are no more than 3 consecutive LNA units, such as no more than 2 consecutive LNA units, and wherein the 5′ nucleotide is a LNA unit and the 3′ nucleotide, such as the 2 3′ nucleotides are LNA units. In some embodiments, the LNA oligomer consists or comprises of alternating 5′-LNA-DNA-3′ nucleotides, optionally with the terminal (5′ and or 3′) two nucleotides being LNA units.

In some embodiments, the oligomer, such as the mixmer described above is 12-16 nucleotides in length, such as 13, 14 or 15 nucleotides.

In some embodiments, the oligomer, such as the mixmer described above is a phosphorothioate oligomer.

### Internucleotide Linkages

The monomers of the oligomers described herein are coupled together via linkage groups. Suitably, each monomer is linked to the 3′ adjacent monomer via a linkage group.

The person having ordinary skill in the art would understand that, in the context of the present invention, the 5′ monomer at the end of an oligomer does not comprise a 5′ linkage group, although it may or may not comprise a 5′ terminal group.

The terms “linkage group” or “internucleotide linkage” are intended to mean a group capable of covalently coupling together two nucleotides. Specific and preferred examples include phosphate groups and phosphorothioate groups.

The nucleotides of the oligomer of the invention or contiguous nucleotides sequence thereof are coupled together via linkage groups. Suitably each nucleotide is linked to the 3′ adjacent nucleotide via a linkage group.

Suitable internucleotide linkages include those listed within WO2007/031091, for example the internucleotide linkages listed on the first paragraph of page 34 of WO2007/031091 (hereby incorporated by reference).

It is, in some embodiments, preferred to modify the internucleotide linkage from its normal phosphodiester to one that is more resistant to nuclease attack, such as phosphorothioate or boranophosphate—these two, being cleavable by RNase H, also allow that route of antisense inhibition in reducing the expression of the target gene.

Suitable sulphur (S) containing internucleotide linkages as provided herein may be preferred. Phosphorothioate internucleotide linkages are also preferred for improved nuclease resistance and other reasons, such as ease of manufacture.

The oligomers may, however, comprise internucleotide linkages other than phosphorothioate, such as phosphodiester linkages, particularly, for instance when the use of nucleotide analogues such as LNA nucleotides protects the internucleotide linkages from endo-nuclease degradation.

It is recognised that the inclusion of phosphodiester linkages, such as one or two linkages, into an otherwise phosphorothioate oligomer, particularly between or adjacent to nucleotide analogue units modify the bioavailability and/or bio-distribution of an oligomer—see WO2008/053314, hereby incorporated by reference.

In some embodiments, such as the embodiments referred to above, where suitable and not specifically indicated, all remaining linkage groups are either phosphodiester or phosphorothioate, or a mixture thereof.

In some embodiments all the internucleotide linkage groups are phosphorothioate.

When referring to specific gapmer oligonucleotide sequences, such as those provided herein it will be understood that, in various embodiments, when the linkages are phosphorothioate linkages, alternative linkages, such as those disclosed herein may be used, for example phosphate (phosphodiester) linkages may be used, particularly for linkages between nucleotide analogues, such as LNA, units. Likewise, when referring to specific gapmer oligonucleotide sequences, such as those provided herein, when the C (cytosine) residues are annotated as 5′ methyl modified cytosine, in various embodiments, one or more of the Cs present in the oligomer may be unmodified C residues.

### Oligomeric Compounds

The oligomers of the invention may, for example, have a sequence selected from the group consisting of SEQ ID NOs 1-83 as shown in Table 1, or a sequence which is a subset of one of the foregoing. In one embodiment, the oligomers are 16mers in which the first, third, fifth, seventh, ninth, eleventh, thirteenth, fifteenth and sixteenth monomer units (starting from the 5′ end) are LNA, the remaining units are DNA, and the linkages are phosphorothioates throughout. In another embodiment, the oligomers are 15mers in which the first, third, fifth, seventh, ninth, eleventh, thirteenth, fourteenth and fifteenth monomer units (starting from the 5′ end) are LNA, the remaining units are DNA, and the linkages are phosphorothioates throughout. In a further embodiment, the oligomers are 12mers in which the first, third, fifth, seventh, ninth, eleventh and twelfth monomer units (starting from the 5′ end) are LNA, the remaining units are DNA, and the linkages are phosphorothioates throughout.

### Conjugates

In the context of this disclosure, the term “conjugate” is intended to indicate a heterogeneous molecule formed by the covalent attachment (“conjugation”) of the oligomer as described herein to one or more non-nucleotide, or non-polynucleotide moieties. Examples of non-nucleotide or non- polynucleotide moieties include macromolecular agents such as proteins, fatty acid chains, sugar residues, glycoproteins, polymers, or combinations thereof. Typically proteins may be antibodies for a target protein. Typical polymers may be polyethylene glycol.

Therefore, in various embodiments, the oligomer of the invention may comprise both a polynucleotide region which typically consists of a contiguous sequence of nucleotides, and a further non-nucleotide region. When referring to the oligomer of the invention consisting of a contiguous nucleotide sequence, the compound may comprise non-nucleotide components, such as a conjugate component.

In various embodiments of the invention the oligomeric compound is linked to ligands/conjugates, which may be used, e.g. to increase the cellular uptake of oligomeric compounds. WO2007/031091 provides suitable ligands and conjugates, which are hereby incorporated by reference.

The invention also provides for a conjugate comprising the compound according to the invention as herein described, and at least one non-nucleotide or non-polynucleotide moiety covalently attached to said compound. Therefore, in various embodiments where the compound of the invention consists of a specified nucleic acid or nucleotide sequence, as herein disclosed, the compound may also comprise at least one non-nucleotide or non-polynucleotide moiety (e.g. not comprising one or more nucleotides or nucleotide analogues) covalently attached to said compound.

Conjugation (to a conjugate moiety) may enhance the activity, cellular distribution or cellular uptake of the oligomer of the invention. Such moieties include, but are not limited to, antibodies, polypeptides, lipid moieties such as a cholesterol moiety, cholic acid, a thioether, e.g. hexyl-s-tritylthiol, a thiocholesterol, an aliphatic chain, e.g., dodecandiol or undecyl residues, a phospholipids, e.g., di-hexadecyl-rac-glycerol or triethylammonium 1,2-di-o-hexadecyl-rac-glycero-3-h-phosphonate, a polyamine or a polyethylene glycol chain, an adamantane acetic acid, a palmityl moiety, an octadecylamine or hexylamino-carbonyl-oxycholesterol moiety.

The oligomers of the invention may also be conjugated to active drug substances, for example, aspirin, ibuprofen, a sulfa drug, an antidiabetic, an antibacterial or an antibiotic.

In certain embodiments the conjugated moiety is a sterol, such as cholesterol.

In various embodiments, the conjugated moiety comprises or consists of a positively charged polymer, such as a positively charged peptides of, for example from 1-50, such as 2-20 such as 3-10 amino acid residues in length, and/or polyalkylene oxide such as polyethylglycol (PEG) or polypropylene glycol—see WO 2008/034123, hereby incorporated by reference. Suitably the positively charged polymer, such as a polyalkylene oxide may be attached to the oligomer of the invention via a linker such as the releasable inker described in WO 2008/034123.

By way of example, the following conjugate moieties may be used in the conjugates of the invention:

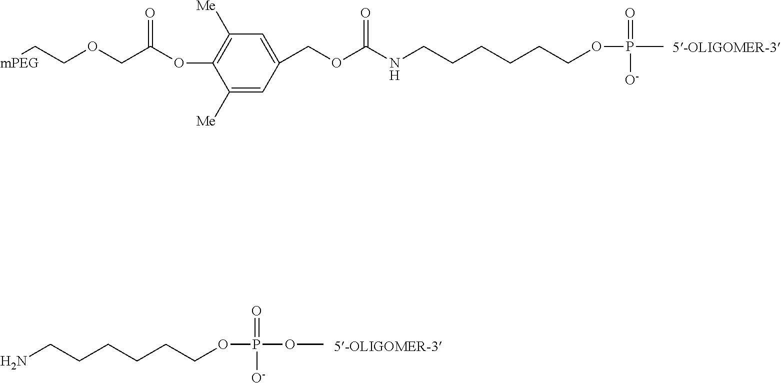

Activated Oligomers

The term “activated oligomer,” as used herein, refers to an oligomer of the invention that is covalently linked (i.e., functionalized) to at least one functional moiety that permits covalent linkage of the oligomer to one or more conjugated moieties, i.e., moieties that are not themselves nucleic acids or monomers, to form the conjugates herein described. Typically, a functional moiety will comprise a chemical group that is capable of covalently bonding to the oligomer via, e.g., a 3′-hydroxyl group or the exocyclic NH₂ group of the adenine base, a spacer that is preferably hydrophilic and a terminal group that is capable of binding to a conjugated moiety (e.g., an amino, sulfhydryl or hydroxyl group). In some embodiments, this terminal group is not protected, e.g., is an NH₂ group. In other embodiments, the terminal group is protected, for example, by any suitable protecting group such as those described in “Protective Groups in Organic Synthesis” by Theodora W Greene and Peter G M Wuts, 3rd edition (John Wiley & Sons, 1999). Examples of suitable hydroxyl protecting groups include esters such as acetate ester, aralkyl groups such as benzyl, diphenylmethyl, or triphenylmethyl, and tetrahydropyranyl. Examples of suitable amino protecting groups include benzyl, alpha-methylbenzyl, diphenylmethyl, triphenylmethyl, benzyloxycarbonyl, tert-butoxycarbonyl, and acyl groups such as trichloroacetyl or trifluoroacetyl. In some embodiments, the functional moiety is self-cleaving. In other embodiments, the functional moiety is biodegradable. See e.g., U.S. Pat. No. 7,087,229, which is incorporated by reference herein in its entirety.

In some embodiments, oligomers of the invention are functionalized at the 5′ end in order to allow covalent attachment of the conjugated moiety to the 5′ end of the oligomer. In other embodiments, oligomers of the invention can be functionalized at the 3′ end. In still other embodiments, oligomers of the invention can be functionalized along the backbone or on the heterocyclic base moiety. In yet other embodiments, oligomers of the invention can be functionalized at more than one position independently selected from the 5′ end, the 3′ end, the backbone and the base.

In some embodiments, activated oligomers of the invention are synthesized by incorporating during the synthesis one or more monomers that is covalently attached to a functional moiety. In other embodiments, activated oligomers of the invention are synthesized with monomers that have not been functionalized, and the oligomer is functionalized upon completion of synthesis. In some embodiments, the oligomers are functionalized with a hindered ester containing an aminoalkyl linker, wherein the alkyl portion has the formula (CH₂)_{w}, wherein w is an integer ranging from 1 to 10, preferably about 6, wherein the alkyl portion of the alkylamino group can be straight chain or branched chain, and wherein the functional group is attached to the oligomer via an ester group (—O—C(O)—(CH₂)_{w}NH).

In other embodiments, the oligomers are functionalized with a hindered ester containing a (CH₂)_{w}-sulfhydryl (SH) linker, wherein w is an integer ranging from 1 to 10, preferably about 6, wherein the alkyl portion of the alkylamino group can be straight chain or branched chain, and wherein the functional group attached to the oligomer via an ester group (—O—C(O)—(CH₂)_{w}SH)

In some embodiments, sulfhydryl-activated oligonucleotides are conjugated with polymer moieties such as polyethylene glycol or peptides (via formation of a disulfide bond).

Activated oligomers containing hindered esters as described above can be synthesized by any method known in the art, and in particular by methods disclosed in PCT Publication No. WO 2008/034122 and the examples therein, which is incorporated herein by reference in its entirety.

In still other embodiments, the oligomers of the invention are functionalized by introducing sulfhydryl, amino or hydroxyl groups into the oligomer by means of a functionalizing reagent substantially as described in U.S. Pat. Nos. 4,962,029 and 4,914,210, i.e., a substantially linear reagent having a phosphoramidite at one end linked through a hydrophilic spacer chain to the opposing end which comprises a protected or unprotected sulfhydryl, amino or hydroxyl group. Such reagents primarily react with hydroxyl groups of the oligomer. In some embodiments, such activated oligomers have a functionalizing reagent coupled to a 5′-hydroxyl group of the oligomer. In other embodiments, the activated oligomers have a functionalizing reagent coupled to a 3′-hydroxyl group. In still other embodiments, the activated oligomers of the invention have a functionalizing reagent coupled to a hydroxyl group on the backbone of the oligomer. In yet further embodiments, the oligomer of the invention is functionalized with more than one of the functionalizing reagents as described in U.S. Pat. Nos. 4,962,029 and 4,914,210, incorporated herein by reference in their entirety. Methods of synthesizing such functionalizing reagents and incorporating them into monomers or oligomers are disclosed in U.S. Pat. Nos. 4,962,029 and 4,914,210.

In some embodiments, the 5′-terminus of a solid-phase bound oligomer is functionalized with a dienyl phosphoramidite derivative, followed by conjugation of the deprotected oligomer with, e.g., an amino acid or peptide via a Diels-Alder cycloaddition reaction.

In various embodiments, the incorporation of monomers containing 2′-sugar modifications, such as a 2′-carbamate substituted sugar or a 2′-(O-pentyl-N-phthalimido)-deoxyribose sugar into the oligomer facilitates covalent attachment of conjugated moieties to the sugars of the oligomer. In other embodiments, an oligomer with an amino-containing linker at the 2′-position of one or more monomers is prepared using a reagent such as, for example, 5′-dimethoxytrityl-2′-O-(e-phthalimidylaminopentyl)-2′-deoxyadenosine-3′-N,N-diisopropyl-cyanoethoxy phosphoramidite. See, e.g., Manoharan, et al., Tetrahedron Letters, 1991, 34, 7171.

In still further embodiments, the oligomers of the invention may have amine-containing functional moieties on the nucleobase, including on the N6 purine amino groups, on the exocyclic N2 of guanine, or on the N4 or 5 positions of cytosine. In various embodiments, such functionalization may be achieved by using a commercial reagent that is already functionalized in the oligomer synthesis.

Some functional moieties are commercially available, for example, heterobifunctional and homobifunctional linking moieties are available from the Pierce Co. (Rockford, Ill.). Other commercially available linking groups are 5′-Amino-Modifier C6 and 3′-Amino-Modifier reagents, both available from Glen Research Corporation (Sterling, Va.). 5′-Amino-Modifier C6 is also available from ABI (Applied Biosystems Inc., Foster City, Calif.) as Aminolink-2, and 3′-Amino-Modifier is also available from Clontech Laboratories Inc. (Palo Alto, Calif.).

### Compositions

The oligomers of the invention may be used in pharmaceutical formulations and compositions. Suitably, such compositions comprise a pharmaceutically acceptable diluent, carrier, salt or adjuvant. WO/2007/031091 provides suitable and preferred pharmaceutically acceptable diluent, carrier and adjuvants—which are hereby incorporated by reference. Suitable dosages, formulations, administration routes, compositions, dosage forms, combinations with other therapeutic agents, pro-drug formulations are also provided in WO/2007/031091—which is hereby incorporated by reference.

### Applications

The oligomers of the invention may be utilized as research reagents for, for example, diagnostics, therapeutics and prophylaxis. In research, such oligomers may also be used to specifically modulate splicing of SMN2 mRNA to facilitate functional analysis of the roles of various splice products.

In diagnostics the oligomers may be used to detect and quantitate SMN2 expression in cell and tissues by northern blotting, in-situ hybridization or similar techniques.

For therapeutics, an animal or a human, suspected of having a disease or disorder, which can be treated by modulating the expression of SMN, or of particular SMN2 mRNA splice products, is treated by administering oligomeric compounds in accordance with this invention. Further provided are methods of treating a human suspected of having or being prone to a disease or condition, associated with aberrant expression of SMN, including expression of aberrant SMN splice products, by administering a therapeutically or prophylactically effective amount of one or more of the oligomers or compositions of the invention. The oligomer, a conjugate or a pharmaceutical composition according to the invention is typically administered in an effective amount.

The invention also provides for a method for treating a disorder as referred to herein said method comprising administering a compound according to the invention as herein described, and/or a conjugate according to the invention, and/or a pharmaceutical composition according to the invention to a patient in need thereof.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 ug to 10 g per kg of body weight, and may be given once or more daily, weekly, monthly or yearly, or even in a single dose per lifetime or as needed. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 ug to 100 g per kg of body weight, once or more daily, to once every 20 years.

### Medical Indications

The oligomers and other compositions according to the invention can be used for the treatment of conditions associated with overexpression, undesired or abnormal levels (particularly high levels as might be due to overaccumulation) or expression of a mutated or otherwise aberrant version of SMN.

The invention further provides use of a compound of the invention in the manufacture of a medicament for the treatment of a disease, disorder or condition as referred to herein.

Generally stated, one aspect of the invention is directed to a method of treating a human subject suffering from or susceptible to conditions associated with undesired or abnormal levels of SMN, comprising administering to the human subject a therapeutically effective amount of an oligomer targeted to SMN2 that comprises one or more LNA units. The oligomer, a conjugate or a pharmaceutical composition according to the invention is typically administered in an effective amount.

The disease or disorder, as referred to herein, may, in some embodiments, be associated with a mutation in the SMN2 gene or a gene whose protein product is associated with or interacts with SMN. Therefore, in some embodiments, the target pre-mRNA is a mutated form of the SMN2 sequence.

The disease or disorder may be associated with aberrant splicing of SMN2, and therefore in some embodiments the oligomer is designed to modulate splicing of the SMN2 mRNA.

The methods of the invention are preferably employed for treatment or prophylaxis against diseases caused by abnormal or undesired levels of SMN, or by aberrant SMN mRNA splice products.

Alternatively stated, in some embodiments, the invention is furthermore directed to a method for modulating abnormal or undesired levels of SMN, e.g., higher than desired levels of SMN, or of particular SMN mRNA splice products, said method comprising administering a oligomer of the invention, or a conjugate of the invention or a pharmaceutical composition of the invention, to a human subject in need thereof.

The invention also relates to an oligomer, a composition or a conjugate as defined herein for use as a medicament. Moreover, the invention relates to a method of treating a subject suffering from a disease or condition such as those referred to herein. A patient who is in need of treatment is a patient suffering from or likely to suffer from the disease or disorder.

In some embodiments, the term ‘treatment’ as used herein refers to both treatment of an existing disease (e.g. a disease or disorder as herein referred to), or prevention of a disease, i.e. prophylaxis. It will therefore be recognised that treatment as referred to herein may, in some embodiments, be prophylactic.

### EMBODIMENTS

1. An oligomer of from 10 to 30 nucleotides in length which comprises at least one Locked Nucleic Acid (LNA) unit and does not elicit RNAse H activity, and wherein the oligomer further comprises a nucleobase sequence of from 10 to 30 nucleobases in length, wherein said nucleobase sequence is at least 80% complementary to a region corresponding to nucleotides 26231-26300, 31881-31945, or 32111-32170 of Genbank Accession No. NG_{—}008728 (SEQ ID NO: 167) or a naturally occurring variant thereof, and which modulates splicing of SMN2 mRNA resulting in an increase in levels of the full length SMN2 mRNA transcript.
2. The oligomer according to embodiment 1 wherein said nucleobase sequence is at least 80% complementary to a region corresponding to nucleotides 26231-26246, 26274-26300, 31890-31905, 31918-31945 or 32115-32162 of Genbank Accession No. NG_{—}008728 (SEQ ID NO: 167).
3. The oligomer according to embodiment 1 wherein said oligomer is at least 80% complementary to nucleotides 26231-26300 of Genbank Accession No. NG_{—}008728 (SEQ ID NO: 167).
4. The oligomer according to embodiment 1 wherein the nucleobase sequence of the oligomer is at least 80% identical to the sequence of SEQ ID NO: 1, 2, 3-16, 19-20, 22, 24-34, 35-38, 40, 41, 45-49, 60-80 or 83.
5. The oligomer according to embodiment 1 wherein the nucleobase sequence of the oligomer has the sequence of SEQ ID NO: 1, 5, 9, 11, 12, 26, 27, 28, 29, 30, 34, 40, 53-59, 62, 63, 65, 66, 69-77 or 79.
6. The oligomer according to embodiment 1 wherein modulation of splicing is an increase in amount of the full length SMN2 transcript to greater than 110% of control, greater than 120% of control, greater than 130% of control, greater than 140% of control, greater than 150% of control greater than 160% of control, greater than 170% of control, greater than 180% of control, greater than 190% of control, or greater than 200% of control.
7. The oligomer according to embodiment 1 wherein the nucleotide sequence is from 12 to 16 nucleotides in length.
8. The oligomer according to embodiment 8 which is a mixmer.
9. A conjugate comprising the oligomer according to embodiment 1 and at least one non-nucleotide or non-polynucleotide moiety covalently attached to said oligomer.
10. The oligomer according to embodiment 1, or the conjugate according to embodiment 9, for use as a medicament, such as for the treatment of spinal muscular atrophy.
11. The oligomer of embodiment 10 wherein the spinal muscular atrophy is Type I, Type II or Type III spinal muscular atrophy.
12. A pharmaceutical composition comprising the oligomer according to embodiment 1, or the conjugate according to embodiment 9, and a pharmaceutically acceptable diluent, carrier, salt or adjuvant.
13. A method of treating spinal muscular atrophy, said method comprising administering an effective amount of an oligomer according to embodiment 1, or a conjugate according to embodiment 9, or a pharmaceutical composition according to embodiment 12, to a patient suffering from or believed likely to suffer from spinal muscular atrophy.
15. A method for modulating splicing of SMN2 mRNA in a human cell expressing SMN2 mRNA, said method comprising administering an oligomer according to embodiment 1, or a conjugate according to embodiment 9, or a pharmaceutical composition of embodiment 12, to said human cell wherein said splicing of SMN2 RNA in said human cell is modulated and the ratio of full length SMN2 mRNA to truncated SMN2 mRNA is increased.

### EXAMPLES

### Example 1

### Design of Oligonucleotides

In accordance with the present invention, a series of oligonucleotides was designed to target the human SMN2 genomic sequence (Genbank accession no. NG_{—}008728). These are chimeric oligonucleotides having beta-D-oxy LNA units at some positions (uppercase) and DNA units at other positions (lowercase), as shown in Table 1. The oligonucleotides were targeted to various regions of the genomic sequence as indicated. “Target site” indicates the nucleotide number of the first (5′-most) nucleotide on Genbank Acc. No. NG_{—}008728 to which the oligonucleotide is complementary. In Table 1, all internucleoside linkages are phosphorothioate linkages and all LNA-cytosines (uppercase) are 5-methylcytosines.

TABLE 1
Antisense oligonucleotide sequences targeted to human SMN2

| | | Target | | | | |
|---|---|---|---|---|---|---|
| Seq | Length | site on | | Target | Oligo Sequence | Seq |
| ID No | (bases) | NG_008728 | Base Sequence (5′-3′) | Region | and modifications (5′-3′)* | ID No |
| 1 | 16 | 26231 | GCTGAGTGATTACTTA | Intron 6 | GcTgAgTgAtTaCtTA | 84 |
| | | | | (SD6) | | |
| 2 | 16 | 26233 | ATGCTGAGTGATTACT | Intron 6 | AtGcTgAgTgAtTaCT | 85 |
| | | | | (SD6) | | |
| 3 | 16 | 26235 | AGATGCTGAGTGATTA | Intron 6 | AgAtGcTgAgTgAtTA | 86 |
| | | | | (SD6) | | |
| 4 | 16 | 26237 | AAAGATGCTGAGTGAT | Intron 6 | AaAgAtGcTgAgTgAT | 87 |
| | | | | (SD6) | | |
| 5 | 16 | 26239 | GAAAAGATGCTGAGTG | Intron 6 | GaAaAgAtGcTgAgTG | 88 |
| | | | | (SD6) | | |
| 6 | 16 | 26241 | AGGAAAAGATGCTGAG | Intron 6 | AgGaAaAgAtGcTgAG | 89 |
| | | | | (SD6) | | |
| 7 | 16 | 26243 | TCAGGAAAAGATGCTG | Intron 6 | TcAgGaAaAgAtGcTG | 90 |
| | | | | (SD6) | | |
| 8 | 16 | 26245 | TGTCAGGAAAAGATGC | Intron 6 | TgTcAgGaAaAgAtGC | 91 |
| | | | | (SD6) | | |
| 9 | 16 | 26247 | ATTGTCAGGAAAAGAT | Intron 6 | AtTgTcAgGaAaAaAT | 92 |
| | | | | (SD6) | | |
| 10 | 16 | 26249 | AAATTGTCAGGAAAAG | Intron 6 | AaAtTgTcAgGaAaAG | 93 |
| | | | | (SD6) | | |
| 11 | 16 | 26251 | AAAAATTGTCAGGAAA | Intron 6 | AaAaAtTgTcAgGaAA | 94 |
| | | | | (SD6) | | |
| 12 | 16 | 26253 | AAAAAAATTGTCAGGA | Intron 6 | AaAaAaAtTgTcAgGA | 95 |
| | | | | (SD6) | | |
| 13 | 16 | 26255 | ACAAAAAAATTGTCAG | Intron 6 | AcAaAaAaAaTgTcAG | 96 |
| | | | | (SD6) | | |
| 14 | 16 | 26257 | CTACAAAAAAATTGTC | Intron 6 | CtAcAaAaAaAtTgTC | 97 |
| | | | | (SD6) | | |
| 15 | 16 | 26259 | AACTACAAAAAAATTG | Intron 6 | AaCtAcAaAaAaAtTG | 98 |
| | | | | (SD6) | | |
| 16 | 16 | 26261 | ATAACTACAAAAAAAT | Intron 6 | AtAaCtAcAaAaAaAT | 99 |
| | | | | (SD6) | | |
| 17 | 16 | 26262 | CATAACTACAAAAAAA | Intron 6 | CaTaAcTaCaAaAaAA | 100 |
| | | | | (SD6) | | |
| 18 | 16 | 26264 | CACATAACTACAAAAA | Intron 6 | CaCaTaAcTaCaAaAA | 101 |
| | | | | (SD6) | | |
| 19 | 16 | 26266 | GTCACATAACTACAAA | Intron 6 | GtCaCaTaAcTaCaAA | 102 |
| | | | | (SD6) | | |
| 20 | 16 | 26268 | AAGTCACATAACTACA | Intron 6 | AaGtCaCaTaAcTaCA | 103 |
| | | | | (SD6) | | |
| 21 | 12 | 26268 | CACATAACTACA | Intron 6 | CaCaTaAcTaCA | 104 |
| | | | | (SD6) | | |
| 22 | 16 | 26270 | CAAAGTCACATAACTA | Intron 6 | CaAaGtCaCaTaAcTA | 105 |
| | | | | (SD6) | | |
| 23 | 12 | 26270 | GTCACATAACTA | Intron 6 | GtCaCaTaAcTA | 106 |
| | | | | (SD6) | | |
| 24 | 16 | 26272 | AACAAAGTCACATAAC | Intron 6 | AaCaAaGtCaCaTaAC | 107 |
| | | | | (SD6) | | |
| 25 | 12 | 26272 | AAGTCACATAAC | Intron 6 | AaGtCaCaTaAC | 108 |
| | | | | (SD6) | | |
| 26 | 16 | 26274 | AAAACAAAGTCACATA | Intron 6 | AaAaCaAaGtCaCaTA | 109 |
| | | | | (SD6) | | |
| 27 | 12 | 26274 | CAAAGTCACATA | Intron 6 | CaAaGtCaCaTA | 110 |
| | | | | (SD6) | | |
| 28 | 16 | 26276 | ACAAAACAAAGTCACA | Intron 6 | AcAaAaCaAaGtCaCA | 111 |
| | | | | (SD6) | | |
| 29 | 12 | 26276 | AACAAAGTCACA | Intron 6 | AaCaAaGtCaCA | 112 |
| | | | | (SD6) | | |
| 30 | 16 | 26278 | TTACAAAACAAAGTCA | Intron 6 | TtAcAaAaCaAaGtCA | 113 |
| | | | | (SD6) | | |
| 31 | 16 | 26280 | ATTTACAAAACAAAGT | Intron 6 | AtTtAcAaAaCaAaGT | 114 |
| | | | | (SD6) | | |
| 32 | 16 | 26282 | AAATTTACAAAACAAA | Intron 6 | AaAtTtAcAaAaCaAA | 115 |
| | | | | (SD6) | | |
| 33 | 16 | 26284 | ATAAATTTACAAAACA | Intron 6 | AtAaAtTtAcAaAaCA | 116 |
| | | | | (SD6) | | |
| 34 | 16 | 26285 | TATAAATTTACAAAAC | Intron 6 | TaTaAaTtTaCaAaAC | 117 |
| | | | | (SD6) | | |
| 35 | 16 | 31881 | GACATTTTACTTATTT | Intron 6 | GaCaTtTtAcTtAtTT | 118 |
| | | | | (ISS-E1) | | |
| 36 | 16 | 31883 | AAGACATTTTACTTAT | Intron 6 | AaGaCaTtTtAcTtAT | 119 |
| | | | | (ISS-E1) | | |
| 37 | 16 | 31885 | ACAAGACATTTTACTT | Intron 6 | AcAaGaCaTtTtAcTT | 120 |
| | | | | (ISS-E1) | | |
| 38 | 16 | 31887 | TCACAAGACATTTTAC | Intron 6 | TcAcAaGaCaTtTtAC | 121 |
| | | | | (ISS-E1) | | |
| 39 | 16 | 31889 | TTTCACAAGACATTTT | Intron 6 | TtTcAcAaGaCaTtTT | 122 |
| | | | | (ISS-E1) | | |
| 40 | 16 | 31890 | GTTTCACAAGACATTT | Intron 6 | GtTtCaCaAgAcAtTT | 123 |
| | | | | (ISS-E1) | | |
| 41 | 16 | 31891 | TGTTTCACAAGACATT | Intron 6 | TgTtTcAcAaGaCaTT | 124 |
| | | | | (ISS-E1) | | |
| 42 | 16 | 31892 | TTGTTTCACAAGACAT | Intron 6 | TtGtTtCaCaAgAcAT | 125 |
| | | | | (ISS-E1) | | |
| 43 | 16 | 31894 | TTTTGTTTCACAAGAC | Intron 6 | TtTtGtTtCaCaAgAC | 126 |
| | | | | (ISS-E1) | | |
| 44 | 16 | 31896 | CATTTTGTTTCACAAG | Intron 6 | CaTtTtGtTtCaCaAG | 127 |
| | | | | (ISS-E1) | | |
| 45 | 16 | 31898 | AGCATTTTGTTTCACA | Intron6 | AgCaTtTtGtTtCaCA | 128 |
| | | | | (ISS-E1) | | |
| 46 | 16 | 31900 | AAAGCATTTTGTTTCA | Intron 6 | AaAgCaTtTtGtTtCA | 129 |
| | | | | (ISS-E1) | | |
| 47 | 16 | 31903 | TAAAAAGCATTTTGTT | Intron 6 | TaAaAaGcAtTtTgTT | 130 |
| | | | | (ISS-E1) | | |
| 48 | 16 | 31905 | GTTAAAAAGCATTTTG | Intron 6 | GtTaAaAaGcAtTtTG | 131 |
| | | | | (ISS-E1) | | |
| 49 | 16 | 31907 | ATGTTAAAAAGCATTT | Intron 6 | AtGtTaAaAaGcAtTT | 132 |
| | | | | (ISS-E1) | | |
| 50 | 16 | 31910 | TGGATGTTAAAAAGCA | Intron 6 | TgGaTgTtAaAaAgCA | 133 |
| | | | | (ISS-E1) | | |
| 51 | 16 | 31912 | TATGGATGTTAAAAAG | Intron 6 | TaTgGaTgTtAaAaAG | 134 |
| | | | | (ISS-E1) | | |
| 52 | 16 | 31915 | TTATATGGATGTTAAA | Intron 6 | TtAtAtGgAtGtTaAA | 135 |
| | | | | (ISS-E1) | | |
| 53 | 16 | 31918 | GCTTTATATGGATGTT | Intron 6 | GcTtTaTaTgGaTgTT | 136 |
| | | | | (ISS-E1) | | |
| 54 | 16 | 31920 | TAGCTTTATATGGATG | Intron 6 | TaGcTtTaTaTgGaTG | 137 |
| | | | | (ISS-E1) | | |
| 55 | 16 | 31922 | GATAGCTTTATATGGA | Intron 6 | GaTaGcTtTaTaTgGA | 138 |
| | | | | (ISS-E1) | | |
| 56 | 16 | 31924 | TAGATAGCTTTATATG | Intron 6 | TaGaTaGcTtTaTaTG | 139 |
| | | | | (ISS-E1) | | |
| 57 | 16 | 31926 | TATAGATAGCTTTATA | Intron 6 | TaTaGaTaGcTtTaTA | 140 |
| | | | | (ISS-E1) | | |
| 58 | 16 | 31928 | TATATAGATAGCTTTA | Intron 6 | TaTaTaGaTaGcTtTA | 141 |
| | | | | (ISS-E1) | | |
| 59 | 16 | 31930 | TATATATAGATAGCTT | Intron 6 | TaTaTaTaGaTaGcTT | 142 |
| | | | | (ISS-E1) | | |
| 60 | 16 | 32111 | AAAAACATTTGTTTTC | Intron 7 | AaAaAcAtTtGtTtTC | 143 |
| | | | | (ISS/ISE-E2) | | |
| 61 | 16 | 32113 | TCAAAAACATTTGTTT | Intron 7 | TcAaAaAcAtTtGtTT | 144 |
| | | | | (ISS/ISE-E2) | | |
| 62 | 16 | 32115 | GTTCAAAAACATTTGT | Intron 7 | GtTcAaAaAcAtTtGT | 145 |
| | | | | (ISS/ISE-E2) | | |
| 63 | 16 | 32117 | ATGTTCAAAAACATTT | Intron 7 | AtGtTcAaAaAcAtTT | 146 |
| | | | | (ISS/ISE-E2) | | |
| 64 | 16 | 32119 | AAATGTTCAAAAACAT | Intron 7 | AaAtGtTcAaAaAcAT | 147 |
| | | | | (ISS/ISE-E2) | | |
| 65 | 16 | 32121 | TTAAATGTTCAAAAAC | Intron 7 | TtAaAtGtTcAaAaAC | 148 |
| | | | | (ISS/ISE-E2) | | |
| 66 | 16 | 32123 | TTTTAAATGTTCAAAA | Intron 7 | TtTtAaAtGtTcAaAA | 149 |
| | | | | (ISS/ISE-E2) | | |
| 67 | 16 | 32125 | GTTTTTAAATGTTCAA | Intron 7 | GtTtTtAaAtGtTcAA | 150 |
| | | | | (ISS/ISE-E2) | | |
| 68 | 16 | 32127 | AAGTTTTTAAATGTTC | Intron 7 | AaGtTtTtAaAtGtTC | 151 |
| | | | | (ISS/ISE-E2) | | |
| 69 | 16 | 32129 | TGAAGTTTTTAAATGT | Intron 7 | TgAaGtTtTtAaAtGT | 152 |
| | | | | (ISS/ISE-E2) | | |
| 70 | 16 | 32130 | CTGAAGTTTTTAAATG | Intron 7 | CtGaAgTtTtTaAaTG | 153 |
| | | | | (ISS/ISE-E2) | | |
| 71 | 16 | 32131 | TCTGAAGTTTTTAAAT | Intron 7 | TcTgAaGtTtTtAaAT | 154 |
| | | | | (ISS/ISE-E2) | | |
| 72 | 16 | 32133 | CATCTGAAGTTTTTAA | Intron 7 | CaTcTgAaGtTtTtAA | 155 |
| | | | | (ISS/ISE-E2) | | |
| 73 | 16 | 32135 | AACATCTGAAGTTTTT | Intron 7 | AaCaTcTgAaGtTtTT | 156 |
| | | | | (ISS/ISE-E2) | | |
| 74 | 16 | 32137 | CTAACATCTGAAGTTT | Intron 7 | CtAaCaTcTgAaGtTT | 157 |
| | | | | (ISS/ISE-E2) | | |
| 75 | 16 | 32139 | TTCTAACATCTGAAGT | Intron 7 | TtCtAaCaTcTgAaGT | 158 |
| | | | | (ISS/ISE-E2) | | |
| 76 | 16 | 32141 | CTTTCTAACATCTGAA | Intron 7 | CtTtCtAaCaTcTgAA | 159 |
| | | | | (ISS/ISE-E2) | | |
| 77 | 16 | 32143 | AACTTTCTAACATCTG | Intron 7 | AaCtTtCtAaCaTcTG | 160 |
| | | | | (ISS/ISE-E2) | | |
| 78 | 16 | 32145 | TCAACTTTCTAACATC | Intron 7 | TcAaCtTtCtAaCaTC | 161 |
| | | | | (ISS/ISE-E2) | | |
| 79 | 16 | 32147 | TTTCAACTTTCTAACA | Intron 7 | TtTcAaCtTtCtAaCA | 162 |
| | | | | (ISS/ISE-E2) | | |
| 80 | 16 | 32148 | CTTTCAACTTTCTAAC | Intron 7 | CtTtCaAcTtTcTaAC | 163 |
| | | | | (ISS/ISE-E2) | | |
| 81 | 16 | 32149 | CCTTTCAACTTTCTAA | Intron 7 | CcTtTcAaCtTtCtAA | 164 |
| | | | | (ISS/ISE-E2) | | |
| 82 | 16 | 32153 | TTAACCTTTCAACTTT | Intron 7 | TtAaCcTtTcAaCtTT | 165 |
| | | | | (ISS/ISE-E2) | | |
| 83 | 16 | 32155 | CATTAACCTTTCAACT | Intron 7 | CaTtAaCcTtTcAaCT | 166 |
| | | | | (ISS/ISE-E2) | | |

*Oligo sequence and modifications: Capital letters are beta-D-oxy LNA nucleosides, lower
case letters are DNA nucleosides. LNA cytosines are optionally 5-methyl cytosine LNA.
Internucleoside linkages are phosphorothioate.

### Example 2

### In Vitro Model: Cell Culture

The effect of antisense oligonucleotides on target nucleic acid expression can be tested in any of a variety of cell types provided that the target nucleic acid is present at measurable levels. The target can be expressed endogenously or by transient or stable transfection of a nucleic acid encoding said target. The expression level of target nucleic acid can be routinely determined using, for example, Northern blot analysis, real-time PCR, ribonuclease protection assays. The following cell types are provided for illustrative purposes, but other cell types can be routinely used, provided that the target is expressed in the cell type chosen.

Cells were cultured in the appropriate medium as described below and maintained at 37° C. at 95-98% humidity and 5% CO₂. Cells were routinely passaged 2-3 times weekly.

SMA1 Cells:

A human SMA1 patient fibroblast cell line (Catalog ID No: GM03813, Coriell Institute for Medical Research, Camden, N.J.) was cultured in Eagle's Minimum Essential Medium (#M5650, Sigma), 2 mM Glutamine (AQ, #G8541, Sigma) and non-essential amino acids (11140-035, Invitrogen) with 10% fetal bovine serum (Biochrom, BCHS0115) and 0.25 μg/ml Gentamycin (G1397, Sigma), This cell line expresses SMN2 but no SMN1 and therefore is representative of the situation in an SMA patient.

### Example 3

### In Vitro Model: Treatment with Antisense Oligonucleotide Using Lipid Transfection

The SMA1 cell line listed in Example 2 was treated with oligonucleotide using the cationic liposome formulation LipofectAMINE 2000 (#11668-019, Invitrogen) as transfection vehicle. Cells were seeded in 6-well cell culture plates (NUNC, #) together with lipofectamine/oligonucleotide mix. Oligos were used at 25 nM final concentration. Formulation of oligo-lipid complexes were carried out essentially as described by the manufacturer using serum-free OptiMEM (#51985, Gibco) and a final lipid concentration of 2.5 μg/mL LipofectAMINE 2000. Transfection was followed by total RNA preparation as described in subsequent examples after 24 hours (RNeasy Mini Kit, #74106, Qiagen,), reverse transcription (M-MLV reverse transcriptase and random decamers, #2044, #5722G, Ambion) and real time PCR using two custom-designed TaqMan gene expression assays (#Applied Biosystems, AI39QW5, AI5I03D) to detect either full length or short transcripts with skipped exon 7. GAPDH was used as a normalizer.

Results are given in Example 7 below (Table 2).

### Example 4

### In Vitro Model: Extraction of RNA and cDNA Synthesis

### Total RNA Isolation and First Strand Synthesis

Total RNA was extracted from cells transfected as described above and using the Qiagen RNeasy kit (#74106, Qiagen) according to the manufacturer's instructions. First strand synthesis was performed using MMLV-Reverse Transcriptase (#2044, Ambion) and Random decamer primer (#5722G, Ambion) reagents from Ambion according to the manufacturer's instructions.

For each sample 0.3-0.4 μg total RNA was adjusted to (10.8 μl) with RNase free H₂O and mixed with 2 μl random decamers (50 μM) and 4 μl dNTP mix (2.5 mM each dNTP) and heated to 70° C. for 3 min after which the samples were rapidly cooled on ice. After cooling the samples on ice, 2 μl 10× Buffer RT, 1 μl MMLV Reverse Transcriptase (100 U/μl) and 0.25 μl RNase inhibitor (10 U/μl) was added to each sample, followed by incubation at 42° C. for 60 min, heat inactivation of the enzyme at 95° C. for 10 min and then the sample was cooled to 4° C.

### Example 5

### In Vitro Model: Analysis of Oligonucleotide Modulation of SMN2 RNA Splicing by Real-Time PCR

Antisense modulation of SMN2 expression can be assayed in a variety of ways known in the art. For example, SMN2 mRNA levels can be quantitated by, e.g., Northern blot analysis, competitive polymerase chain reaction (PCR), or real-time PCR. Real-time quantitative PCR is presently preferred. RNA analysis can be performed on total cellular RNA or mRNA.

Methods of RNA isolation and RNA analysis such as Northern blot analysis is routine in the art and is taught in, for example, Current Protocols in Molecular Biology, John Wiley and Sons. Real-time quantitative (PCR) can be conveniently accomplished using the commercially available Multi-Color Real Time PCR Detection System, available from Applied Biosystems.

Real-Time Quantitative PCR Analysis of SMN2 mRNA Levels

The sample content of human full length and exon7-skipped SMN2 mRNAs was quantified using custom designed human SMN ABI Prism TaqMan Assays (full length #AI5I03D, exon 7-skipped #AI39QW5, Applied Biosystems) according to the manufacturer's instructions. Glyceraldehyde-3-phosphate dehydrogenase (GAPDH) mRNA quantity was used as an endogenous control for normalizing any variance in sample preparation.

The sample content of human GAPDH mRNA was quantified using the human GAPDH ABI Prism Pre-Developed TaqMan Assay Reagent (#4310884E, Applied Biosystems) according to the manufacturer's instructions.

Real-time Quantitative PCR is a technique well known in the art and is taught in for example Heid et al. Real time quantitative PCR, Genome Research (1996), 6: 986-994.

Real Time PCR:

The cDNA from the first strand synthesis performed as described in Example 5 was diluted 5 times, and analyzed by real time quantitative PCR using Taqman 7500 FAST or 7900 FAST from Applied Biosystems. The primers and probe were mixed with 2× Taqman Fast Universal PCR master mix (2×) (#4352042, Applied Biosystems) and added to 4 μl cDNA to a final volume of 10 μl. Each sample was analysed in duplicate. Assaying 2 fold dilutions of a cDNA that had been prepared on material purified from a cell line expressing the RNA of interest generated standard curves for the assays. Sterile H₂O was used instead of cDNA for the no template control. PCR program: 95° C. for 20 seconds, followed by 40 cycles of 95° C., 3 seconds, 60° C., 30 seconds. Relative quantities of target mRNA sequence were determined from the calculated Threshold cycle using the Applied Biosystems Fast System SDS Software Version 1.3.1.21. or SDS Software Version 2.3.

### Example 6

### In Vitro Analysis: Antisense Modulation of Human SMN2 mRNA Splicing by Oligonucleotide Compounds Targeted to SMN2 Region 5′ of SD6 (Intron 6)

Oligonucleotides presented in Table 1 were evaluated in the SMA1 cell line for their potential to modulate SMN2 mRNA splicing at an oligo concentration of 25 nM using lipid transfection. These oligonucleotides are targeted to the region 5′ of splice donor 6 (SD6) in intron 6 of SMN2, a region not previously targeted in the literature. Results are shown in Table 2.

Table 2: Antisense Modulation of human SMN2 splicing -

The data in Table 2 are presented as percentage down-regulation relative to mock transfected cells at 25 nM in SMA1 cells. Oligonucleotide sequences and modifications are shown in Table 1.

TABLE 2
Fine tiling of LNA antisense oligonucleotides
in SMN2 region 5′ of SD6 (intron 6)

| | Target | % Δ7 | % Full length |
|---|---|---|---|
| Seq | site on | SMN2 transcript | SMN2 transcript |
| ID No | NG_008728 | (exon 7 skipped) | (exon 7 included) |
| 1 | 26231 | 89 | 181 |
| 2 | 26233 | 50 | 62 |
| 3 | 26235 | 87 | 113 |
| 4 | 26237 | 45 | 116 |
| 5 | 26239 | 19 | 154 |
| 6 | 26241 | 142 | 123 |
| 7 | 26243 | 143 | 105 |
| 8 | 26245 | 49 | 131 |
| 9 | 26247 | 43 | 166 |
| 10 | 26249 | 50 | 144 |
| 11 | 26251 | 37 | 149 |
| 12 | 26253 | 27 | 156 |
| 13 | 26255 | 30 | 141 |
| 14 | 26257 | 52 | 127 |
| 15 | 26259 | 78 | 108 |
| 16 | 26261 | 67 | 107 |
| 17 | 26262 | 65 | 85 |
| 18 | 26264 | 79 | 83 |
| 19 | 26266 | 31 | 107 |
| 20 | 26268 | 31 | 131 |
| 21 | 26268 | 202 | 86 |
| 22 | 26270 | 18 | 118 |
| 23 | 26270 | 184 | 50 |
| 24 | 26272 | 9 | 132 |
| 25 | 26272 | 17 | 105 |
| 26 | 26274 | 17 | 298 |
| 27 | 26274 | 10 | 221 |
| 28 | 26276 | 19 | 201 |
| 29 | 26276 | 10 | 170 |
| 30 | 26278 | 46 | 205 |
| 31 | 26280 | 86 | 130 |
| 32 | 26282 | 85 | 143 |
| 33 | 26284 | 74 | 101 |
| 34 | 26285 | 175 | 208 |
| control | | 100 | 100 |

Oligonucleotides that result in a level of full length SMN2 mRNA greater than 100% of control are preferred. As can be seen from Table 2, oligonucleotides having SEQ ID NOs: 1, 2, 3-16, 19-20, 22 and 24-34 achieve such an increase in full-length SMN2 transcript. These presently preferred oligomers are targeted to nucleotides 26231-26300 of Genbank Acc. No. NG_{—}008728.

Oligonucleotides of SEQ ID NOs: 1, 5, 9, 11, 12, 26, 27, 28, 29, 30 and 34 demonstrated an increase to about 150% or greater of full length SMN2 mRNA expression compared to control (in this experiment, mock transfected cells), along with a decrease in SMN2Δ7 mRNA expression in these experiments and are therefore particularly preferred. As will be understood, these oligos are causing splice switching to increase SMN2 exon 7 inclusion and decrease the levels of the poorly functional truncated SMN2 Δ7 transcript. These particularly preferred compounds are targeted to nucleotide positions 26231-26246 and 26274-26300 on NG_{—}008728.

Also preferred are oligonucleotides based on the illustrated antisense oligo sequences, for example varying the length (shorter or longer) and/or nucleobase content (e.g. the type and/or proportion of analogue units), which also provide good modulation of SMN2 expression in favor of the full length transcript, preferably at least 150% full length compared to control.

### Example 7

### In Vitro Analysis: Antisense Modulation of Human SMN2 mRNA Splicing by Oligonucleotide Compounds Targeted to SMN2 Region ISS-E1 (Intron 6)

Oligonucleotides presented in Table 1 were evaluated in the SMA1 cell line for their potential to modulate SMN2 mRNA splicing at an oligo concentration of 25 nM using lipid transfection. Results are shown in Table 3.

TABLE 3
Fine tiling of LNA-antisense oligonucleotides
in SMN2 region ISS-E1(intron 6)
The data in Table 3 are presented as percentage
down-regulation relative to mock transfected
cells at 25 nM in SMA1 cells. Oligonucleotide
sequences and modifications are shown in Table 1.

| | % Δ7 | % Full length |
|---|---|---|
| Seq | SMN2 transcript | SMN2 transcript |
| ID No | (exon 7 skipped) | (exon 7 included) |
| 35 | 94 | 155 |
| 36 | 103 | 169 |
| 37 | 120 | 143 |
| 38 | 180 | 116 |
| 39 | 236 | 68 |
| 40 | 548 | 415 |
| 41 | 155 | 128 |
| 42 | 177 | 42 |
| 43 | 298 | 20 |
| 44 | 223 | 20 |
| 45 | 149 | 120 |
| 46 | 148 | 136 |
| 47 | 223 | 150 |
| 48 | 115 | 139 |
| 49 | 47 | 123 |
| 50 | 80 | 128 |
| 51 | 110 | 165 |
| 52 | 77 | 134 |
| 53 | 26 | 290 |
| 54 | 60 | 470 |
| 55 | 22 | 245 |
| 56 | 28 | 404 |
| 57 | 29 | 220 |
| 58 | 50 | 425 |
| 59 | 25 | 233 |
| control | 100 | 100 |

Oligomers that result in a level of full length SMN2 mRNA greater than 100% of control (as shown in Table 3) are preferred. As can be seen from the table, oligomers having SEQ ID NOs: 35-38, 40, 41, and 45-49 achieve such an increase in full-length SMN2 transcript. These oligomers are targeted to nucleotide positions 31881-31945 of Genbank Acc. No. NG_{—}008728. Oligomers of SEQ ID NOs: 53-59, targeted to nucleotide positions 31890-31905 and 31918-31945 of Genbank Acc. No. NG_{—}008728, demonstrated an increase to about 200% or greater of full length SMN2 mRNA expression compared to control (in this experiment, mock transfected cells) along with a decrease in SMN2Δ7 mRNA expression in these experiments and are therefore particularly preferred. As will be understood, these oligos are causing splice switching to increase SMN2 exon 7 inclusion and decrease the levels of the poorly functional truncated SMN2 Δ7 transcript. The oligonucleotide of SEQ ID NO 40 is also particularly preferred because it demonstrated an increase to greater than 200% of full length SMN2 in this experiment compared to control, along with an increase in SMNΔ7. These particularly preferred oligomers are targeted to nucleotide positions 31890-31905 and 31918-31945 on NG_{—}008728.

Also preferred are oligomers based on the illustrated antisense oligomer sequences, for example varying the length (shorter or longer) and/or nucleobase content (e.g. the type and/or proportion of analogue units), which also provide comparable (to at least about 200% compared to control) modulation of SMN2 splicing to increase SMN2 exon 7 inclusion (increase in full length SMN2 transcript).

### Example 8

### In Vitro Analysis: Antisense Modulation of Human SMN2 mRNA Splicing by Oligonucleotide Compounds Targeted to SMN2 Region ISE/ISS-E2 (Intron 7)

Oligomers presented in Table 1 were evaluated in the SMA1 cell line for their potential to modulate SMN2 mRNA splicing at an oligo concentration of 25 nM using lipid transfection. Results are shown in Table 4.

TABLE 4
Fine tiling of LNA-antisense oligonucleotides
in SMN2 region ISE/ISS-E2 (intron 7)
The data in Table 4 are presented as percentage
down-regulation relative to mock transfected
cells at 25 nM in SMA1 cells. Oligonucleotide
sequences and modifications are shown in Table 1.

| | % Δ7 | % Full length |
|---|---|---|
| Seq | SMN2 transcript | SMN2 transcript |
| ID No | (exon 7 skipped) | (exon 7 included) |
| 60 | 80 | 138 |
| 61 | 155 | 179 |
| 62 | 36 | 279 |
| 63 | 85 | 215 |
| 64 | 66 | 162 |
| 65 | 29 | 205 |
| 66 | 163 | 232 |
| 67 | 59 | 186 |
| 68 | 193 | 144 |
| 69 | 53 | 247 |
| 70 | 15 | 309 |
| 71 | 14 | 227 |
| 72 | 13 | 336 |
| 73 | 15 | 261 |
| 74 | 16 | 282 |
| 75 | 22 | 291 |
| 76 | 73 | 261 |
| 77 | 67 | 272 |
| 78 | 74 | 125 |
| 79 | 263 | 244 |
| 80 | 200 | 107 |
| 81 | 274 | 87 |
| 82 | 331 | 46 |
| 83 | 119 | 124 |
| control | 100 | 100 |

Oligomers that result in a level of full length SMN2 mRNA greater than 100% of control (as shown in Table 4) are preferred. As can be seen from the table, oligomers having SEQ ID NOs: 60-80 and 83 achieve such an increase in full-length SMN2 transcript. These oligomers are targeted to nucleotide positions 31211-32170 of Genbank Acc. No. NG_{—}008728. Oligomers of SEQ ID NOs: 62, 63, 65, 66 and 69-77, targeted to nucleotide positions 32115-32162 of Genbank Acc. No. NG_{—}008728, demonstrated an increase to about 200% or greater of full length SMN2 mRNA expression compared to control cells (in this experiment, mock transfected cells) along with a decrease in SMN2Δ7 mRNA expression in these experiments and are therefore particularly preferred. As will be understood, these oligos are causing splice switching to increase SMN2 exon 7 inclusion and decrease the levels of the poorly functional truncated SMN2 Δ7 transcript. The oligonucleotide of SEQ ID NO 79 is also particularly preferred because it demonstrated at least about a 200% increase in both full length and SMNΔ7 transcripts compared to control in this experiment. These particularly preferred oligomers are targeted to nucleotide positions 32115-32162 on NG_{—}008728.

Also preferred are oligonucleotides based on the illustrated antisense oligomer sequences, for example varying the length (shorter or longer) and/or nucleobase content (e.g. the type and/or proportion of analogue units), which also provide comparable modulation of SMN2 splicing to increase SMN2 exon 7 inclusion (increase in full length SMN2 transcript).

All of the compositions and methods described and claimed herein can be made and executed without undue experimentation in light of the present disclosure. While the compositions and methods of this invention have been described in terms of preferred embodiments, it will be apparent to those of skill in the art that variations may be applied to the compositions and methods. All such similar substitutes and modifications apparent to those skilled in the art are deemed to be within the spirit and scope of the invention as defined by the appended claims.

1. An oligomer of 10-30 nucleotides in length, comprising at least one LNA unit, wherein the nucleobase sequence of the oligomer is at least 80% complementary to a corresponding region of nucleotides 26231-26300, 31881-31945, or 32111-32170 of Genbank Accession No. NG_{—}008728 (SEQ ID NO: 167) or a naturally occurring variant thereof.

2. The LNA oligomer according to claim 1, wherein said nucleobase sequence is at least 80% complementary to a region corresponding to nucleotides 26231-26246, 26274-26300, 31890-31905, 31918-31945 or 32115-32162 of Genbank Accession No. NG_{—}008728 (SEQ ID NO: 167).

3. The oligomer according to claim 1 wherein said oligomer is at least 80% complementary to nucleotides 26231-26300 of Genbank Accession No. NG_{—}008728 (SEQ ID NO: 167).

4. The oligomer according to claim 1 wherein the nucleobase sequence of the oligomer is at least 80% identical to the sequence of SEQ ID NO: 1, 2, 3-16, 19-20, 22, 24-34, 35-38, 40, 41, 45-49, 60-80 or 83.

5. The oligomer according to claim 1 wherein the nucleobase sequence of the oligomer has the sequence of SEQ ID NO: 1, 5, 9, 11, 12, 26, 27, 28, 29, 30, 34, 40, 53-59, 62, 63, 65, 66, 69-77 or 79.

6. The oligomer according to claim 1, wherein the oligomer modulates splicing of SMN2 mRNA resulting in an increase in levels of the full length SMN2 mRNA transcript.

7. The oligomer according to claim 1, wherein the oligomer does do not elicit RNAse H cleavage of the nucleic acid target.

8. The oligomer according to claim 1, wherein the oligomer comprises of only LNA and DNA nucleotides.

9. The oligomer according to claim 1, which has fewer than 4 contiguous DNA units, such as fewer than 3 contiguous DNA units, such as fewer than 2 contiguous DNA units.

10. The oligomer according to claim 1, wherein the oligomer comprises of LNA and DNA nucleotides, wherein there are no more than 3 consecutive LNA units, such as no more than 2 consecutive LNA units, and wherein the 5′ nucleotide is a LNA unit and the 3′ nucleotide, such as the 2 3′ nucleotides are LNA units.

11. The oligomer according to claim 1, wherein the oligomer is 12-16 nucleotides in length.

12. The oligomer according to claim 1, wherein the oligomer is a phosphorothioate oligomer.

13. A conjugate comprising the oligomer according to claim 1 and at least one non-nucleotide or non-polynucleotide moiety covalently attached to said oligomer.

14. The oligomer according to claim 1, or the conjugate according to claim 13, for use as a medicament, such as for the treatment of spinal muscular atrophy, such as Type I, Type II or Type III spinal muscular atrophy.

15. A pharmaceutical composition comprising the oligomer according to claim 1, and a pharmaceutically acceptable diluent, carrier, salt or adjuvant.

16. An in vitro method for modulating splicing of SMN2 mRNA in a human cell expressing SMN2 mRNA, said method comprising administering an oligomer according to claim 1, to said human cell wherein said splicing of SMN2 RNA in said human cell is modulated and the ratio of full length SMN2 mRNA to truncated SMN2 mRNA is increased.

17. A pharmaceutical composition comprising the conjugate of claim 13, and a pharmaceutically acceptable diluent, carrier, salt or adjuvant.

18. An in vitro method for modulating splicing of SMN2 mRNA in a human cell expressing SMN2 mRNA, said method comprising administering the conjugate of claim 13, to said human cell wherein said splicing of SMN2 RNA in said human cell is modulated and the ratio of full length SMN2 mRNA to truncated SMN2 mRNA is increased.

19. An in vitro method for modulating splicing of SMN2 mRNA in a human cell expressing SMN2 mRNA, said method comprising administering an oligomer according to the pharmaceutical composition of claim 15, to said human cell wherein said splicing of SMN2 RNA in said human cell is modulated and the ratio of full length SMN2 mRNA to truncated SMN2 mRNA is increased.
